(19) United States
(12) Patent Application Publication
(10) Pub. No.: US 2024/0360452 A1
(43) Pub. Date: Oct. 31, 2024

(54) METHODS AND MEANS FOR EFFICIENT SKIPPING OF AT LEAST ONE OF THE FOLLOWING EXONS OF THE HUMAN DUCHENNE MUSCULAR DYSTROPHY GENE: 43, 46, 50-53

(71) Applicants: BioMarin Technologies B.V., Amsterdam (NL); Academisch Ziekenhuis Leiden, Leiden (NL)

(72) Inventor: Judith Christina Theodora van Deutekom, Dordrecht (NL)

(21) Appl. No.: 18/771,603

(22) Filed: Jul. 12, 2024

Related U.S. Application Data
(63) Continuation of application No. 17/129,117, filed on Dec. 21, 2020.
Application No. 17/129,117 is a continuation of application No. 16/024,558, filed on Jun. 29, 2018, now Pat. No. 10,876,114.
Application No. 16/024,558 is a continuation of application No. 15/289,053, filed on Oct. 7, 2016, now abandoned.
Application No. 15/289,053 is a continuation of application No. 14/631,686, filed on Feb. 25, 2015, now Pat. No. 9,499,818.
Application No. 14/631,686 is a continuation of application No. 13/094,571, filed on Apr. 26, 2011, now abandoned.
Application No. 13/094,571 is a continuation of application No. PCT/NL2009/050113, filed on Mar. 11, 2009.
Application No. PCT/NL2009/050113 is a continuation-in-part of application No. PCT/NL2008/050673, filed on Oct. 27, 2008.

(51) Int. Cl. C12N 15/113; A61K 31/56; A61K 31/57; A61K 31/573; A61K 31/58; A61K 31/7088; A61K 38/17; A61K 45/06; A61K 48/00

(52) U.S. Cl. CPC C12N 15/113; A61K 31/56; A61K 31/57; A61K 31/573; A61K 31/58; A61K 31/7088; A61K 38/1719; A61K 45/06; A61K 48/00; C12N 2310/11; C12N 2310/111; C12N 2310/31; C12N 2310/313; C12N 2310/314; C12N 2310/315; C12N 2310/3181; C12N 2310/321; C12N 2310/3231; C12N 2310/3233; C12N 2310/346; C12N 2320/31; C12N 2320/33

(57) ABSTRACT

The invention relates to a method wherein a molecule is used for inducing and/or promoting skipping of at least one of exon 43, exon 46, or exons 50-53 of the DMD pre-mRNA in a patient, the method comprising providing the patient with the molecule. The invention also relates to the molecule as such.

### RELATED APPLICATIONS

This application is a continuation of U.S. application Ser. No. 17/129,117, filed Dec. 21, 2020, which is a continuation of U.S. application Ser. No. 16/024,558, filed Jun. 29, 2018, now U.S. Pat. No. 10,876,114, which is a continuation of U.S. application Ser. No. 15/289,053, filed Oct. 7, 2016, which is a continuation of U.S. application Ser. No. 14/631,686, filed Feb. 25, 2015, now U.S. Pat. No. 9,499,818, which is a continuation of U.S. application Ser. No. 13/094,571, filed Apr. 26, 2011, which is a continuation of PCT/NL2009/050113, filed Mar. 11, 2009, which is a continuation-in-part of PCT/NL2008/050673, filed Oct. 27, 2008. The disclosures of each of the above-referenced applications are incorporated by reference herein in their entirety.

### SEQUENCE LISTING

This application contains a Sequence Listing, which is being submitted herewith as an XML filed named “010507USCN5_SL.xml”, created on Jul. 12, 2024, size 680,971 bytes, which is incorporated by reference herein in its entirety.

### FIELD

The invention relates to the field of genetics, more specifically human genetics. The invention in particular relates to modulation of splicing of the human Duchenne Muscular Dystrophy pre-mRNA.

### BACKGROUND

Myopathies are disorders that result in functional impairment of muscles. Muscular dystrophy (MD) refers to genetic diseases that are characterized by progressive weakness and degeneration of skeletal muscles. Duchenne muscular dystrophy (DMD) and Becker muscular dystrophy (BMD) are the most common childhood forms of muscular dystrophy. They are recessive disorders and because the gene responsible for DMD and BMD resides on the X-chromosome, mutations mainly affect males with an incidence of about 1 in 3500 boys.

DMD and BMD are caused by genetic defects in the DMD gene encoding dystrophin, a muscle protein that is required for interactions between the cytoskeleton and the extracellular matrix to maintain muscle fiber stability during contraction. DMD is a severe, lethal neuromuscular disorder resulting in a dependency on wheelchair support before the age of 12 and DMD patients often die before the age of thirty due to respiratory- or heart failure. In contrast, BMD patients often remain ambulatory until later in life, and have near normal life expectancies. DMD mutations in the DMD gene are characterized by frame shifting insertions or deletions or nonsense point mutations, resulting in the absence of functional dystrophin. BMD mutations in general keep the reading frame intact, allowing synthesis of a partly functional dystrophin.

During the last decade, specific modification of splicing in order to restore the disrupted reading frame of the dystrophin transcript has emerged as a promising therapy for Duchenne muscular dystrophy (DMD) (van Ommen, van Deutekom, Aartsma-Rus, Curr Opin Mol Ther. 2008; 10(2):140-9, Yokota, Duddy, Partidge, Acta Myol. 2007; 26(3):179-84, van Deutekom et al., N Engl J Med. 2007; 357(26):2677-86).

Using antisense oligonucleotides (AONs) interfering with splicing signals the skipping of specific exons can be induced in the DMD pre-mRNA, thus restoring the open reading frame and converting the severe DMD into a milder BMD phenotype (van Deutekom et al. Hum Mol Genet. 2001; 10: 1547-54; Aartsma-Rus et al., Hum Mol Genet 2003; 12(8):907-14.). In vivo proof-of-concept was first obtained in the mdx mouse model, which is dystrophin-deficient due to a nonsense mutation in exon 23. Intramuscular and intravenous injections of AONs targeting the mutated exon 23 restored dystrophin expression for at least three months (Lu et al. Nat Med. 2003; 8: 1009-14; Lu et al., Proc Natl Acad Sci US A. 2005; 102(1):198-203). This was accompanied by restoration of dystrophin-associated proteins at the fiber membrane as well as functional improvement of the treated muscle. In vivo skipping of human exons has also been achieved in the hDMD mouse model, which contains a complete copy of the human DMD gene integrated in chromosome 5 of the mouse (Bremmer-Bout et al. Molecular Therapy. 2004; 10: 232-40; 't Hoen et al. J Biol Chem. 2008; 283: 5899-907).

Recently, a first-in-man study was successfully completed where an AON inducing the skipping of exon 51 was injected into a small area of the tibialis anterior muscle of four DMD patients. Novel dystrophin expression was observed in the majority of muscle fibers in all four patients treated, and the AON was safe and well tolerated (van Deutekom et al. N Engl J Med. 2007; 357: 2677-86).

### BRIEF DESCRIPTION OF THE DRAWINGS

FIG. 1. In human control myotubes, a series of AONs (PS237, PS238, and PS240; SEQ ID NO 65, 66, 16 respectively) targeting exon 43 was tested at 500 nM. PS237 (SEQ ID NO 65) reproducibly induced highest levels of exon 43 skipping. (M: DNA size marker; NT: non-treated cells)

FIG. 2. In myotubes from a DMD patient with an exon 45 deletion, a series of AONs (PS177, PS179, PS181, and PS182; SEQ ID NO 91, 70, 110, and 117 respectively) targeting exon 46 was tested at two different concentrations (50 and 150 nM). PS182 (SEQ ID NO 117) reproducibly induced highest levels of exon 46 skipping. (M: DNA size marker)

FIG. 3. In human control myotubes, a series of AONs (PS245, PS246, PS247, and PS248; SEQ ID NO 167, 165, 166, and 127 respectively) targeting exon 50 was tested at 500 nM. PS248 (SEQ ID NO 127) reproducibly induced highest levels of exon 50 skipping. (M: DNA size marker; NT: non-treated cells).

FIG. 4. In human control myotubes, two novel AONs (PS232 and PS236; SEQ ID NO 246 and 299 respectively) targeting exon 52 were tested at two different concentrations (200 and 500 nM) and directly compared to a previously described AON (52-1). PS236 (SEQ ID NO 299) reproducibly induced highest levels of exon 52 skipping. (M: DNA size marker; NT: non-treated cells).

### DETAILED DESCRIPTION

### Method

In a first aspect, the present invention provides a method for inducing, and/or promoting skipping of at least one of exons 43, 46, 50-53 of the DMD pre-mRNA in a patient, preferably in an isolated cell of a patient, the method comprising providing said cell and/or said patient with a molecule that binds to a continuous stretch of at least 8 nucleotides within said exon. It is to be understood that said method encompasses an in vitro, in vivo or ex vivo method.

Accordingly, a method is provided for inducing and/or promoting skipping of at least one of exons 43, 46, 50-53 of DMD pre-mRNA in a patient, preferably in an isolated cell of said patient, the method comprising providing said cell and/or said patient with a molecule that binds to a continuous stretch of at least 8 nucleotides within said exon.

As defined herein a DMD pre-mRNA preferably means the pre-mRNA of a DMD gene of a DMD or BMD patient.

A patient is preferably intended to mean a patient having DMD or BMD as later defined herein or a patient susceptible to develop DMD or BMD due to his or her genetic background. In the case of a DMD patient, an oligonucleotide used will preferably correct one mutation as present in the DMD gene of said patient and therefore will preferably create a DMD protein that will look like a BMD protein: said protein will preferably be a functional dystrophin as later defined herein. In the case of a BMD patient, an oligonucleotide as used will preferably correct one mutation as present in the BMD gene of said patient and therefore will preferably create a dystrophin which will be more functional than the dystrophin which was originally present in said BMD patient.

Exon skipping refers to the induction in a cell of a mature mRNA that does not contain a particular exon that is normally present therein. Exon skipping is performed by providing a cell expressing the pre-mRNA of said mRNA with a molecule capable of interfering with essential sequences such as for example the splice donor of splice acceptor sequence that required for splicing of said exon, or a molecule that is capable of interfering with an exon inclusion signal that is required for recognition of a stretch of nucleotides as an exon to be included in the mRNA. The term pre-mRNA refers to a non-processed or partly processed precursor mRNA that is synthesized from a DNA template in the cell nucleus by transcription.

Within the context of the invention, inducing and/or promoting skipping of an exon as indicated herein means that at least 1%, 10%, 20%, 30%, 40%, 50%, 60%, 70%, 80%, 90% or more of the DMD mRNA in one or more (muscle) cells of a treated patient will not contain said exon. This is preferably assessed by PCR as described in the examples.

Preferably, a method of the invention by inducing and/or promoting skipping of at least one of the following exons 43, 46, 50-53 of the DMD pre-mRNA in one or more (muscle) cells of a patient, provides said patient with a functional dystrophin protein and/or decreases the production of an aberrant dystrophin protein in said patient and/or increases the production of a functional dystrophin is said patient.

Providing a patient with a functional dystrophin protein and/or decreasing the production of an aberrant dystrophin protein in said patient is typically applied in a DMD patient. Increasing the production of a functional dystrophin is typically applied in a BMD patient.

Therefore, a preferred method is a method, wherein a patient or one or more cells of said patient is provided with a functional dystrophin protein and/or wherein the production of an aberrant dystrophin protein in said patient is decreased and/or wherein the production of a functional dystrophin is increased in said patient, wherein the level of said aberrant or functional dystrophin is assessed by comparison to the level of said dystrophin in said patient at the onset of the method.

Decreasing the production of an aberrant dystrophin may be assessed at the mRNA level and preferably means that 99%, 90%, 80%, 70%, 60%, 50%, 40%, 30%, 20%, 10%, 5% or less of the initial amount of aberrant dystrophin mRNA, is still detectable by RT PCR. An aberrant dystrophin mRNA or protein is also referred to herein as a non-functional dystrophin mRNA or protein. A non-functional dystrophin protein is preferably a dystrophin protein which is not able to bind actin and/or members of the DGC protein complex. A non-functional dystrophin protein or dystrophin mRNA does typically not have, or does not encode, a dystrophin protein with an intact C-terminus of the protein.

Increasing the production of a functional dystrophin in said patient or in a cell of said patient may be assessed at the mRNA level (by RT-PCR analysis) and preferably means that a detectable amount of a functional dystrophin mRNA is detectable by RT PCR. In another embodiment, 1%, 5%, 10%, 20%, 30%, 40%, 50%, 60%, 70%, 80%, 90% or more of the detectable dystrophin mRNA is a functional dystrophin mRNA. Increasing the production of a functional dystrophin in said patient or in a cell of said patient may be assessed at the protein level (by immunofluorescence and western blot analyses) and preferably means that a detectable amount of a functional dystrophin protein is detectable by immunofluorescence or western blot analysis. In another embodiment, 1%, 5%, 10%, 20%, 30%, 40%, 50%, 60%, 70%, 80%, 90% or more of the detectable dystrophin protein is a functional dystrophin protein.

As defined herein, a functional dystrophin is preferably a wild type dystrophin corresponding to a protein having the amino acid sequence as identified in SEQ ID NO: 1. A functional dystrophin is preferably a dystrophin, which has an actin binding domain in its N terminal part (first 240 amino acids at the N terminus), a cysteine-rich domain (amino acid 3361 till 3685) and a C terminal domain (last 325 amino acids at the C terminus) each of these domains being present in a wild type dystrophin as known to the skilled person. The amino acids indicated herein correspond to amino acids of the wild type dystrophin being represented by SEQ ID NO:1. In other words, a functional dystrophin is a dystrophin which exhibits at least to some extent an activity of a wild type dystrophin. “At least to some extent” preferably means at least 10%, 20%, 30%, 40%, 50%, 60%, 70%, 80%, 90%, 95% or 100% of a corresponding activity of a wild type functional dystrophin. In this context, an activity of a functional dystrophin is preferably binding to actin and to the dystrophin-associated glycoprotein complex (DGC) (Aartsma-Rus A et al, (2006), Entries in the leiden Duchenne Muscular Dystrophy mutation database: an overview of mutation types and paradoxical cases that confirm the reading-frame rule, Muscle Nerve, 34: 135-144). Binding of dystrophin to actin and to the DGC complex may be visualized by either co-immunoprecipitation using total protein extracts or immunofluorescence analysis of cross-sections, from a muscle biopsy, as known to the skilled person.

Individuals or patients suffering from Duchenne muscular dystrophy typically have a mutation in the gene encoding dystrophin that prevent synthesis of the complete protein, i.e of a premature stop prevents the synthesis of the C-terminus. In Becker muscular dystrophy the DMD gene also comprises a mutation compared to the wild type gene, but the mutation does typically not induce a premature stop and the C-terminus is typically synthesized. As a result, a functional dystrophin protein is synthesized that has at least the same activity in kind as the wild type protein, not although not necessarily the same amount of activity. The genome of a BMD individual typically encodes a dystrophin protein comprising the N terminal part (first 240 amino acids at the N terminus), a cysteine-rich domain (amino acid 3361 till 3685) and a C terminal domain (last 325 amino acids at the C terminus) but its central rod shaped domain may be shorter than the one of a wild type dystrophin (Aartsma-Rus A et al, (2006), Entries in the leiden Duchenne Muscular Dystrophy mutation database: an overview of mutation types and paradoxical cases that confirm the reading-frame rule, Muscle Nerve, 34: 135-144). Exon skipping for the treatment of DMD is typically directed to overcome a premature stop in the pre-mRNA by skipping an exon in the rod-shaped domain to correct the reading frame and allow synthesis of remainder of the dystrophin protein including the C-terminus, albeit that the protein is somewhat smaller as a result of a smaller rod domain. In a preferred embodiment, an individual having DMD and being treated by a method as defined herein will be provided a dystrophin which exhibits at least to some extent an activity of a wild type dystrophin. More preferably, if said individual is a Duchenne patient or is suspected to be a Duchenne patient, a functional dystrophin is a dystrophin of an individual having BMD: typically said dystrophin is able to interact with both actin and the DGC, but its central rod shaped domain may be shorter than the one of a wild type dystrophin (Aartsma-Rus A et al, (2006), Entries in the leiden Duchenne Muscular Dystrophy mutation database: an overview of mutation types and paradoxical cases that confirm the reading-frame rule, Muscle Nerve, 34: 135-144). The central rod-shaped domain of wild type dystrophin comprises 24 spectrin-like repeats (Aartsma-Rus A et al, (2006), Entries in the leiden Duchenne Muscular Dystrophy mutation database: an overview of mutation types and paradoxical cases that confirm the reading-frame rule, Muscle Nerve, 34: 135-144). For example, a central rod-shaped domain of a dystrophin as provided herein may comprise 5 to 23, 10 to 22 or 12 to 18 spectrin-like repeats as long as it can bind to actin and to DGC.

A method of the invention may alleviate one or more characteristics of a myogenic or muscle cell of a patient or alleviate one or more symptoms of a DMD patient having a deletion including but not limited to exons 44, 44-46, 44-47, 44-48, 44-49, 44-51, 44-53 (correctable by exon 43 skipping), 19-45, 21-45, 43-45, 45, 47-54, 47-56 (correctable by exon 46 skipping), 51, 51-53, 51-55, 51-57 (correctable by exon 50 skipping), 13-50, 19-50, 29-50, 43-50, 45-50, 47-50, 48-50, 49-50, 50, 52 (correctable by exon 51 skipping), exons 8-51, 51, 53, 53-55, 53-57, 53-59, 53-60, (correctable by exon 52 skipping) and exons 10-52, 42-52, 43-52, 45-52, 47-52, 48-52, 49-52, 50-52, 52 (correctable by exon 53 skipping) in the DMD gene, occurring in a total of 68% of all DMD patients with a deletion (Aartsma-Rus et al., Hum. Mut. 2009).

Alternatively, a method of the invention may improve one or more characteristics of a muscle cell of a patient or alleviate one or more symptoms of a DMD patient having small mutations in, or single exon duplications of exon 43, 46, 50-53 in the DMD gene, occurring in a total of 36% of all DMD patients with a deletion (Aartsma-Rus et al, Hum. Mut. 2009)

Furthermore, for some patients the simultaneous skipping of one of more exons in addition to exon 43, exon 46 and/or exon 50-53 is required to restore the open reading frame, including patients with specific deletions, small (point) mutations, or double or multiple exon duplications, such as (but not limited to) a deletion of exons 44-50 requiring the co-skipping of exons 43 and 51, with a deletion of exons 46-50 requiring the co-skipping of exons 45 and 51, with a deletion of exons 44-52 requiring the co-skipping of exons 43 and 53, with a deletion of exons 46-52 requiring the co-skipping of exons 45 and 53, with a deletion of exons 51-54 requiring the co-skipping of exons 50 and 55, with a deletion of exons 53-54 requiring the co-skipping of exons 52 and 55, with a deletion of exons 53-56 requiring the co-skipping of exons 52 and 57, with a nonsense mutation in exon 43 or exon 44 requiring the co-skipping of exon 43 and 44, with a nonsense mutation in exon 45 or exon 46 requiring the co-skipping of exon 45 and 46, with a nonsense mutation in exon 50 or exon 51 requiring the co-skipping of exon 50 and 51, with a nonsense mutation in exon 51 or exon 52 requiring the co-skipping of exon 51 and 52, with a nonsense mutation in exon 52 or exon 53 requiring the co-skipping of exon 52 and 53, or with a double or multiple exon duplication involving exons 43, 46, 50, 51, 52, and/or 53.

In a preferred method, the skipping of exon 43 is induced, or the skipping of exon 46 is induced, or the skipping of exon 50 is induced or the skipping of exon 51 is induced or the skipping of exon 52 is induced or the skipping of exon 53 is induced. An induction of the skipping of two of these exons is also encompassed by a method of the invention. For example, preferably skipping of exons 50 and 51, or 52 and 53, or 30 43 and 51, or 43 and 53, or 51 and 52. Depending on the type and the identity (the specific exons involved) of mutation identified in a patient, the skilled person will know which combination of exons needs to be skipped in said patient.

In a preferred method, one or more symptom(s) of a DMD or a BMD patient is/are alleviated and/or one or more characteristic(s) of one or more muscle cells from a DMD or a BMD patient is/are improved. Such symptoms or characteristics may be assessed at the cellular, tissue level or on the patient self.

An alleviation of one or more characteristics may be assessed by any of the following assays on a myogenic cell or muscle cell from a patient: reduced calcium uptake by muscle cells, decreased collagen synthesis, altered morphology, altered lipid biosynthesis, decreased oxidative stress, and/or improved muscle fiber function, integrity, and/or survival. These parameters are usually assessed using immunofluorescence and/or histochemical analyses of cross sections of muscle biopsies.

The improvement of muscle fiber function, integrity and/or survival may be assessed using at least one of the following assays: a detectable decrease of creatine kinase in blood, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in Hodgetts et al (Hodgetts S., et al, (2006), Neuromuscular Disorders, 16: 591-602.2006). A detectable decrease in creatine kinase may mean a decrease of 5%, 10%, 20%, 30%, 40%, 50%, 60%, 70%, 80%, 90% or more compared to the concentration of creatine kinase in a same DMD or BMD patient before treatment.

A detectable decrease of necrosis of muscle fibers is preferably assessed in a muscle biopsy, more preferably as described in Hodgetts et al (Hodgetts S., et al (2006), Neuromuscular Disorders, 16: 591-602.2006) using biopsy cross-sections. A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed in a same DMD or BMD patient before treatment.

A detectable increase of the homogeneity of the diameter of a muscle fiber is preferably assessed in a muscle biopsy cross-section, more preferably as described in Hodgetts et al (Hodgetts S., et al, (2006), Neuromuscular Disorders, 16: 591-602.2006). The increase is measured by comparison to the homogeneity of the diameter of a muscle fiber in a same DMD or BMD patient before treatment. An alleviation of one or more symptoms may be assessed by any of the following assays on the patient self: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, improvement of the quality of life. Each of these assays is known to the skilled person. As an example, the publication of Manzur et al. (Manzur A Y et al, (2008), Glucocorticoid corticosteroids for Duchenne muscular dystrophy (review), Wiley publishers, The Cochrane collaboration.) gives an extensive explanation of each of these assays. For each of these assays, as soon as a detectable improvement or prolongation of a parameter measured in an assay has been found, it will preferably mean that one or more symptoms of Duchenne Muscular Dystrophy or Becker Muscular Dystrophy has been alleviated in an individual using a method of the invention. Detectable improvement or prolongation is preferably a statistically significant improvement or prolongation as described in Hodgetts et al (Hodgetts S., et al, (2006), Neuromuscular Disorders, 16: 591-602.2006). Alternatively, the alleviation of one or more symptom(s) of Duchenne Muscular Dystrophy or Becker Muscular Dystrophy may be assessed by measuring an improvement of a muscle fiber function, integrity and/or survival as later defined herein.

A treatment in a method according to the invention may have a duration of at least one week, at least one month, at least several months, at least one year, at least 2, 3, 4, 5, 6 years or more.

Each molecule or oligonucleotide or equivalent thereof as defined herein for use according to the invention may be suitable for direct administration to a cell, tissue and/or an organ in vivo of individuals affected by or at risk of developing DMD or BMD, and may be administered directly in vivo, ex vivo or in vitro. The frequency of administration of a molecule or an oligonucleotide or a composition of the invention may depend on several parameters such as the age of the patient, the mutation of the patient, the number of molecules (dose), the formulation of said molecule. The frequency may be ranged between at least once in a two weeks, or three weeks or four weeks or five weeks or a longer time period.

A molecule or oligonucleotide or equivalent thereof can be delivered as is to a cell. When administering said molecule, oligonucleotide or equivalent thereof to an individual, it is preferred that it is dissolved in a solution that is compatible with the delivery method. For intravenous, subcutaneous, intramuscular, intrathecal and/or intraventricular administration it is preferred that the solution is a physiological salt solution. Particularly preferred for a method of the invention is the use of an excipient that will further enhance delivery of said molecule, oligonucleotide or functional equivalent thereof as defined herein, to a cell and into a cell, preferably a muscle cell. Preferred excipients are defined in the section entitled “pharmaceutical composition”.

In a preferred method of the invention, an additional molecule is used which is able to induce and/or promote skipping of another exon of the DMD pre-mRNA of a patient. Preferably, the second exon is selected from: exon 6, 7, 11, 17, 19, 21, 43, 44, 45, 50, 51, 52, 53, 55, 57, 59, 62, 63, 65, 66, 69, or 75 of the DMD pre-mRNA of a patient. Molecules which can be used are depicted in any one of Table 1 to 7. This way, inclusion of two or more exons of a DMD pre-mRNA in mRNA produced from this pre-mRNA is prevented. This embodiment is further referred to as double- or multi-exon skipping (Aartsma-Rus A, Janson A A, Kaman W E, et al. Antisense-induced multiexon skipping for Duchenne muscular dystrophy makes more sense. Am J Hum Genet 2004; 74(1):83-92, Aartsma-Rus A, Kaman W E, Weij R, den Dunnen J T, van Ommen G J, van Deutekom J C. Exploring the frontiers of therapeutic exon skipping for Duchenne muscular dystrophy by double targeting within one or multiple exons. Mol Ther 2006; 14(3):401-7). In most cases double-exon skipping results in the exclusion of only the two targeted exons from the DMD pre-mRNA. However, in other cases it was found that the targeted exons and the entire region in between said exons in said pre-mRNA were not present in the produced mRNA even when other exons (intervening exons) were present in such region. This multi-skipping was notably so for the combination of oligonucleotides derived from the DMD gene, wherein one oligonucleotide for exon 45 and one oligonucleotide for exon 51 was added to a cell transcribing the DMD gene. Such a set-up resulted in mRNA being produced that did not contain exons 45 to 51. Apparently, the structure of the pre-mRNA in the presence of the mentioned oligonucleotides was such that the splicing machinery was stimulated to connect exons 44 and 52 to each other.

It is possible to specifically promote the skipping of also the intervening exons by providing a linkage between the two complementary oligonucleotides. Hence, in one embodiment stretches of nucleotides complementary to at least two dystrophin exons are separated by a linking moiety. The at least two stretches of nucleotides are thus linked in this embodiment so as to form a single molecule.

In case, more than one compounds or molecules are used in a method of the invention, said compounds can be administered to an individual in any order. In one embodiment, said compounds are administered simultaneously (meaning that said compounds are administered within 10 hours, preferably within one hour). This is however not necessary. In another embodiment, said compounds are administered sequentially.

### Molecule

In a second aspect, there is provided a molecule for use in a method as described in the previous section entitled “Method”. A molecule as defined herein is preferably an oligonucleotide or antisense oligonucleotide (AON).

It was found by the present investigators that any of exon 43, 46, 50-53 is specifically skipped at a high frequency using a molecule that preferably binds to a continuous stretch of at least 8 nucleotides within said exon. Although this effect can be associated with a higher binding affinity of said molecule, compared to a molecule that binds to a continuous stretch of less than 8 nucleotides, there could be other intracellular parameters involved that favor thermodynamic, kinetic, or structural characteristics of the hybrid duplex. In a preferred embodiment, a molecule that binds to a continuous stretch of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides within said exon is used.

In a preferred embodiment, a molecule or an oligonucleotide of the invention which comprises a sequence that is complementary to a part of any of exon 43, 46, 50-53 of DMD pre-mRNA is such that the complementary part is at least 50% of the length of the oligonucleotide of the invention, more preferably at least 60%, even more preferably at least 70%, even more preferably at least 80%, even more preferably at least 90% or even more preferably at least 95%, or even more preferably 98% and most preferably up to 100%. “A part of said exon” preferably means a stretch of at least 8 nucleotides. In a most preferred embodiment, an oligonucleotide of the invention consists of a sequence that is complementary to part of said exon DMD pre-mRNA as defined herein. For example, an oligonucleotide may comprise a sequence that is complementary to part of said exon DMD pre-mRNA as defined herein and additional flanking sequences. In a more preferred embodiment, the length of said complementary part of said oligonucleotide is of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. Preferably, additional flanking sequences are used to modify the binding of a protein to said molecule or oligonucleotide, or to modify a thermodynamic property of the oligonucleotide, more preferably to modify target RNA binding affinity.

A preferred molecule to be used in a method of the invention binds or is complementary to a continuous stretch of at least 8 nucleotides within one of the following nucleotide sequences selected from:

(SEQ ID NO: 2)
5′-AGAUAGUCUACAACAAAGCUCAGGUCGGAUUGACAUUAUUCAUAGCAAGAAG
ACAGCAGCAUUGCAAAGUGCAACGCCUGUGG-3′ for skipping of exon 43;
(SEQ ID NO: 3)
5′-UUAUGGUUGGAGGAAGCAGAUAACAUUGCUAGUAUCCCACUUGAACCUGGAA
AAGAGCAGCAACUAAAAGAAAAGC-3′ for skipping of exon 46;
(SEQ ID NO: 4)
5′-GGCGGUAAACCGUUUACUUCAAGAGCUGAGGGCAAAGCAGCCUGACCUAGCUC
CUGGACUGACCACUAUUGG-3′ for skipping of exon 50;
(SEQ ID NO: 5)
5′-CUCCUACUCAGACUGUUACUCUGGUGACACAACCUGUGGUUACUAAGGAAA
CUGCCAUCUCCAAACUAGAAAUGCCAUCUUCCUUGAUGUUGGAGGUAC-3′
for skipping of exon 51;
(SEQ ID NO: 6)
5′-AUGCAGGAUUUGGAACAGAGGCGUCCCCAGUUGGAAGAACUCAU
UACCGCUGCCCAAAAUUUGAAAAACAAGACCAGCAAUCAAGAGGCU-3′
for skipping of exon 52;
and
(SEQ ID NO: 7)
5′-AAAUGUUAAAGGAUUCAACACAAUGGCUGGAAGCUAAGGAAGAA
GCUGAGCAGGUCUUAGGACAGGCCAGAG-3′ for skipping of exon 53.

Of the numerous molecules that theoretically can be prepared to bind to the continuous nucleotide stretches as defined by SEQ ID NO 2-7 within one of said exons, the invention provides distinct molecules that can be used in a method for efficiently skipping of at least one of exon 43, exon 46 and/or exon 50-53. Although the skipping effect can be addressed to the relatively high density of putative SR protein binding sites within said stretches, there could be other parameters involved that favor uptake of the molecule or other, intracellular parameters such as thermodynamic, kinetic, or structural characteristics of the hybrid duplex.

It was found that a molecule that binds to a continuous stretch comprised within or consisting of any of SEQ ID NO 2-7 results in highly efficient skipping of exon 43, exon 46 and/or exon 50-53 respectively in a cell and/or in a patient provided with this molecule.

Therefore, in a preferred embodiment, a method is provided wherein a molecule binds to a continuous stretch of at least 8, 10, 12, 15, 18, 20, 25, 30, 35, 40, 45, 50 nucleotides within SEQ ID NO 2-7.

In a preferred embodiment for inducing and/or promoting the skipping of any of exon 43, exon 46 and/or exon 50-53, the invention provides a molecule comprising or consisting of an antisense nucleotide sequence selected from the antisense nucleotide sequences depicted in any of Tables 1 to 6. A molecule of the invention preferably comprises or consist of the antisense nucleotide sequence of SEQ ID NO 16, SEQ ID NO 65, SEQ ID NO 70, SEQ ID NO 91, SEQ ID NO 110, SEQ ID NO 117, SEQ ID NO 127, SEQ ID NO 165, SEQ ID NO 166, SEQ ID NO 167, SEQ ID NO 246, SEQ ID NO 299, SEQ ID NO:357.

A preferred molecule of the invention comprises a nucleotide-based or nucleotide or an antisense oligonucleotide sequence of between 8 and 50 nucleotides or bases, more preferred between 10 and 50 nucleotides, more preferred between 20 and 40 nucleotides, more preferred between 20 and 30 nucleotides, such as 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides or 50 nucleotides. A most preferred molecule of the invention comprises a nucleotide-based sequence of 25 nucleotides.

Furthermore, none of the indicated sequences is derived from conserved parts of splice-junction sites. Therefore, said molecule is not likely to mediate differential splicing of other exons from the DMD pre-mRNA or exons from other genes.

In one embodiment, a molecule of the invention is a compound molecule that binds to the specified sequence, or a protein such as an RNA-binding protein or a non-natural zinc-finger protein that has been modified to be able to bind to the corresponding nucleotide sequence on a DMD pre-RNA molecule. Methods for screening compound molecules that bind specific nucleotide sequences are, for example, disclosed in PCT/NL01/00697 and U.S. Pat. No. 6,875,736, which are herein incorporated by reference. Methods for designing RNA-binding Zinc-finger proteins that bind specific nucleotide sequences are disclosed by Friesen and Darby, Nature Structural Biology 5: 543-546 (1998) which is herein incorporated by reference.

A preferred molecule of the invention binds to at least part of the sequence of SEQ ID NO 2:5′-AGAUAGUCUACAACAAAGCUCAGGUCGGAUUGACAUUAUUCAU AGCAAGAAGACAGCAGCAUUGCAAAGUGCAACGCCUGUGG-3′ which is present in exon 43 of the DMD gene. More preferably, the invention provides a molecule comprising or consisting of the antisense nucleotide sequence of SEQ ID NO 8 to SEQ ID NO 69.

In an even more preferred embodiment, the invention provides a molecule comprising or consisting of the antisense nucleotide sequence of SEQ ID NO:16 and/or SEQ ID NO:65. In a most preferred embodiment, the invention provides a molecule comprising or consisting of the antisense nucleotide sequence of SEQ ID NO 65. It was found that this molecule is very efficient in modulating splicing of exon 43 of the DMD pre-mRNA in a muscle cell and/or in a patient.

Another preferred molecule of the invention binds to at least part of the sequence of SEQ ID NO 3: 5′-UUAUGGUUGGAGGAAGCAGAUAACAUUGCUAGUAUCCCACUUG AACCUGGAAAAGAGCAGCAACUAAAAGAAAAGC-3′ which is present in exon 46 of the DMD gene. More preferably, the invention provides a molecule comprising or consisting of the antisense nucleotide sequence of SEQ ID NO 70 to SEQ ID NO 122.

In an even more preferred embodiment, the invention provides a molecule comprising or consisting of the antisense nucleotide sequence of SEQ ID NO 70, SEQ ID NO 91, SEQ ID NO 110, and/or SEQ ID NO117.

In a most preferred embodiment, the invention provides a molecule comprising or consisting of the antisense nucleotide sequence of SEQ ID NO 117. It was found that this molecule is very efficient in modulating splicing of exon 46 of the DMD pre-mRNA in a muscle cell or in a patient.

Another preferred molecule of the invention binds to at least part of the sequence of SEQ ID NO 4: 5′-GGCGGUAAACCGUUUACUUCAAGAGCU GAGGGCAAAGCAGCCUG ACCUAGCUCCUGGACUGACCACUAUUGG-3′ which is present in exon 50 of the DMD gene. More preferably, the invention provides a molecule comprising or consisting of the antisense nucleotide sequence of SEQ ID NO 123 to SEQ ID NO 167 and/or SEQ ID NO 529 to SEQ ID NO 535.

In an even more preferred embodiment, the invention provides a molecule comprising or consisting of the antisense nucleotide sequence of SEQ ID NO 127, or SEQ ID NO 165, or SEQ ID NO 166 and/or SEQ ID NO 167.

In a most preferred embodiment, the invention provides a molecule comprising or consisting of the antisense nucleotide sequence of SEQ ID NO 127. It was found that this molecule is very efficient in modulating splicing of exon 50 of the DMD pre-mRNA in a muscle cell and/or in a patient.

Another preferred molecule of the invention binds to at least part of the sequence of SEQ ID NO 5: 5′-CUCCUACUCAGACUGUUACUCUGGUGACACAACCUGUGGUUACU AAGGAAACUGCCAUCUCCAAACUAGAAAUGCCAUCUUCCUUGAUG UUGGAGGUAC-3′ which is present in exon 51 of the DMD gene. More preferably, the invention provides a molecule comprising or consisting of the antisense nucleotide sequence of SEQ ID NO 168 to SEQ ID NO 241.

Another preferred molecule of the invention binds to at least part of the sequence of SEQ IDNO 6: 5′-AUGCAGGAUUUGGAACAGAGGCGUCCCCAGUUGGAAGAACUCAU UACCGCUGCCCAAAAUUUGAAAAACAAGACCAGCAAUCAAGAGGCU-3′ which is present in exon 52 of the DMD gene. More preferably, the invention provides a molecule comprising or consisting of the antisense nucleotide sequence of SEQ ID NO 242 to SEQ ID NO 310. In an even more preferred embodiment, the invention provides a molecule comprising or consisting of the antisense nucleotide sequence of SEQ ID NO 246 and/or SEQ ID NO 299. In a most preferred embodiment, the invention provides a molecule comprising or consisting of the antisense nucleotide sequence of SEQ ID NO 299. It was found that this molecule is very efficient in modulating splicing of exon 52 of the DMD pre-mRNA in a muscle cell and/or in a patient.

Another preferred molecule of the invention binds to at least part of the sequence of SEQ ID NO 7: 5′-AAAUGUUAAAGGAUUCAACACAAUGGCUGGAAGCUAAGGAAGAA GCUGAGCAGGUCUUAGGACAGGCCAGAG-3′ which is present in exon 53 of the DMD gene. More preferably, the invention provides a molecule comprising or consisting of the antisense nucleotide sequence of SEQ ID NO 311 to SEQ ID NO 358.

In a most preferred embodiment, the invention provides a molecule comprising or consisting of the antisense nucleotide sequence of SEQ ID NO 357. It was found that this molecule is very efficient in modulating splicing of exon 53 of the DMD pre-mRNA in a muscle cell and/or in a patient.

A nucleotide sequence of a molecule of the invention may contain RNA residues, or one or more DNA residues, and/or one or more nucleotide analogues or equivalents, as will be further detailed herein below.

It is preferred that a molecule of the invention comprises one or more residues that are modified to increase nuclease resistance, and/or to increase the affinity of the antisense nucleotide for the target sequence. Therefore, in a preferred embodiment, the antisense nucleotide sequence comprises at least one nucleotide analogue or equivalent, wherein a nucleotide analogue or equivalent is defined as a residue having a modified base, and/or a modified backbone, and/or a non-natural internucleoside linkage, or a combination of these modifications.

In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report demonstrated triplex formation by a morpholino oligonucleotide and, because of the non-ionic backbone, these studies showed that the morpholino oligonucleotide was capable of triplex formation in the absence of magnesium.

It is further preferred that that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In yet a further embodiment, a nucleotide analogue or equivalent of the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkylphosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent of the invention comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O—, S—, or N-alkyl; O—, S—, or N-alkenyl; O—, S— or N-alkynyl; O—, S—, or N-allyl; O-alkyl-O-alkyl, -methoxy, -aminopropoxy; -aminoxy; methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or a deoxyribose or a derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-O, 4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target RNA.

It is understood by a skilled person that it is not necessary for all positions in an antisense oligonucleotide to be modified uniformly. In addition, more than one of the aforementioned analogues or equivalents may be incorporated in a single antisense oligonucleotide or even at a single position within an antisense oligonucleotide. In certain embodiments, an antisense oligonucleotide of the invention has at least two different types of analogues or equivalents.

A preferred antisense oligonucleotide according to the invention comprises a 2′-0 alkyl phosphorothioate antisense oligonucleotide, such as 2′-O-methyl modified ribose (RNA), 2′-O-ethyl modified ribose, 2′-O-propyl modified ribose, and/or substituted derivatives of these modifications such as halogenated derivatives.

A most preferred antisense oligonucleotide according to the invention comprises of 2′-O-methyl phosphorothioate ribose.

A functional equivalent of a molecule of the invention may be defined as an oligonucleotide as defined herein wherein an activity of said functional equivalent is retained to at least some extent. Preferably, an activity of said functional equivalent is inducing exon 43, 46, 50, 51, 52, or 53 skipping and providing a functional dystrophin protein. Said activity of said functional equivalent is therefore preferably assessed by detection of exon 43, 46, 50, 51, 52, or 53 skipping and by quantifying the amount of functional dystrophin protein. A functional dystrophin is herein preferably defined as being a dystrophin able to bind actin and members of the DGC protein complex. The assessment of said activity of an oligonucleotide is preferably done by RT-PCR or by immunofluorescence or Western blot analyses. Said activity is preferably retained to at least some extent when it represents at least 50%, or at least 60%, or at least 70% or at least 80% or at least 90% or at least 95% or more of corresponding activity of said oligonucleotide the functional equivalent derives from. Throughout this application, when the word oligonucleotide is used it may be replaced by a functional equivalent thereof as defined herein.

It will be understood by a skilled person that distinct antisense oligonucleotides can be combined for efficiently skipping any of exon 43, exon 46, exon 50, exon 51, exon 52 and/or exon 53 of the human DMD pre-mRNA. It is encompassed by the present invention to use one, two, three, four, five or more oligonucleotides for skipping one of said exons (i.e., exon, 43, 46, 50, 51, 52, or 53). It is also encompassed to use at least two oligonucleotides for skipping at least two, of said exons. Preferably two of said exons are skipped. More preferably, these two exons are:
43 and 51, or
43 and 53, or
50 and 51, or
51 and 52, or
52 and 53.

The skilled person will know which combination of exons is preferred to be skipped depending on the type, the number and the location of the mutation present in a DMD or BMD patient.

An antisense oligonucleotide can be linked to a moiety that enhances uptake of the antisense oligonucleotide in cells, preferably muscle cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

A preferred antisense oligonucleotide comprises a peptide-linked PMO.

A preferred antisense oligonucleotide comprising one or more nucleotide analogs or equivalents of the invention modulates splicing in one or more muscle cells, including heart muscle cells, upon systemic delivery. In this respect, systemic delivery of an antisense oligonucleotide comprising a specific nucleotide analog or equivalent might result in targeting a subset of muscle cells, while an antisense oligonucleotide comprising a distinct nucleotide analog or equivalent might result in targeting of a different subset of muscle cells. Therefore, in one embodiment it is preferred to use a combination of antisense oligonucleotides comprising different nucleotide analogs or equivalents for inducing skipping of exon 43, 46, 50, 51, 52, or 53 of the human DMD pre-mRNA.

A cell can be provided with a molecule capable of interfering with essential sequences that result in highly efficient skipping of exon 43, exon 46, exon 50, exon 51, exon 52 or exon 53 of the human DMD pre-mRNA by plasmid-derived antisense oligonucleotide expression or viral expression provided by adenovirus- or adeno-associated virus-based vectors. In a preferred embodiment, there is provided a viral-based expression vector comprising an expression cassette that drives expression of a molecule as identified herein. Expression is preferably driven by a polymerase III promoter, such as a U1, a U6, or a U7 RNA promoter. A muscle or myogenic cell can be provided with a plasmid for antisense oligonucleotide expression by providing the plasmid in an aqueous solution. Alternatively, a plasmid can be provided by transfection using known transfection agentia such as, for example, LipofectAMINE™ 2000 (Invitrogen) or polyethyleneimine (PEI; ExGen500 (MBI Fermentas)), or derivatives thereof.

One preferred antisense oligonucleotide expression system is an adenovirus associated virus (AAV)-based vector. Single chain and double chain AAV-based vectors have been developed that can be used for prolonged expression of small antisense nucleotide sequences for highly efficient skipping of exon 43, 46, 50, 51, 52 or 53 of the DMD pre-mRNA.

A preferred AAV-based vector comprises an expression cassette that is driven by a polymerase III-promoter (Pol III). A preferred Pol III promoter is, for example, a U1, a U6, or a U7 RNA promoter.

The invention therefore also provides a viral-based vector, comprising a Pol III-promoter driven expression cassette for expression of one or more antisense sequences of the invention for inducing skipping of exon 43, exon 46, exon 50, exon 51, exon 52 or exon 53 of the human DMD pre-mRNA.

### PHARMACEUTICAL COMPOSITION

If required, a molecule or a vector expressing an antisense oligonucleotide of the invention can be incorporated into a pharmaceutically active mixture or composition by adding a pharmaceutically acceptable carrier.

Therefore, in a further aspect, the invention provides a composition, preferably a pharmaceutical composition comprising a molecule comprising an antisense oligonucleotide according to the invention, and/or a viral-based vector expressing the antisense sequence(s) according to the invention and a pharmaceutically acceptable carrier.

A preferred pharmaceutical composition comprises a molecule as defined herein and/or a vector as defined herein, and a pharmaceutical acceptable carrier or excipient, optionally combined with a molecule and/or a vector as defined herein which is able to induce skipping of exon 6, 7, 11, 17, 19, 21, 43, 44, 45, 50, 51, 52, 53, 55, 57, 59, 62, 63, 65, 66, 69, or 75 of the DMD pre-mRNA. Preferred molecules able to induce skipping of any of these exon are identified in any one of Tables 1 to 7.

Preferred excipients include excipients capable of forming complexes, vesicles and/or liposomes that deliver such a molecule as defined herein, preferably an oligonucleotide complexed or trapped in a vesicle or liposome through a cell membrane. Many of these excipients are known in the art. Suitable excipients comprise polyethylenimine and derivatives, or similar cationic polymers, including polypropyleneimine or polyethylenimine copolymers (PECs) and derivatives, ExGen 500, synthetic amphiphils (SAINT-18), lipofectin™, DOTAP and/or viral capsid proteins that are capable of self-assembly into particles that can deliver such molecule, preferably an oligonucleotide as defined herein to a cell, preferably a muscle cell. Such excipients have been shown to efficiently deliver (oligonucleotide such as antisense) nucleic acids to a wide variety of cultured cells, including muscle cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles.

Polycations such like diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver a molecule or a compound as defined herein, preferably an oligonucleotide across cell membranes into cells.

In addition to these common nanoparticle materials, the cationic peptide protamine offers an alternative approach to formulate a compound as defined herein, preferably an oligonucleotide as colloids. This colloidal nanoparticle system can form so called proticles, which can be prepared by a simple self-assembly process to package and mediate intracellular release of a compound as defined herein, preferably an oligonucleotide. The skilled person may select and adapt any of the above or other commercially available alternative excipients and delivery systems to package and deliver a compound as defined herein, preferably an oligonucleotide for use in the current invention to deliver said compound for the treatment of Duchenne Muscular Dystrophy or Becker Muscular Dystrophy in humans.

In addition, a compound as defined herein, preferably an oligonucleotide could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake into cells and/or the intracellular release of a compound as defined herein, preferably an oligonucleotide from vesicles, e.g. endosomes or lysosomes.

Therefore, in a preferred embodiment, a compound as defined herein, preferably an oligonucleotide are formulated in a medicament which is provided with at least an excipient and/or a targeting ligand for delivery and/or a delivery device of said compound to a cell and/or enhancing its intracellular delivery. Accordingly, the invention also encompasses a pharmaceutically acceptable composition comprising a compound as defined herein, preferably an oligonucleotide and further comprising at least one excipient and/or a targeting ligand for delivery and/or a delivery device of said compound to a cell and/or enhancing its intracellular delivery. It is to be understood that a molecule or compound or oligonucleotide may not be formulated in one single composition or preparation. Depending on their identity, the skilled person will know which type of formulation is the most appropriate for each compound.

In a preferred embodiment, an in vitro concentration of a molecule or an oligonucleotide as defined herein, which is ranged between 0.1 nM and 1 μM is used. More preferably, the concentration used is ranged between 0.3 to 400 nM, even more preferably between 1 to 200 nM. A molecule or an oligonucleotide as defined herein may be used at a dose which is ranged between 0.1 and 20 mg/kg, preferably 0.5 and 10 mg/kg. If several molecules or oligonucleotides are used, these concentrations may refer to the total concentration of oligonucleotides or the concentration of each oligonucleotide added. The ranges of concentration of oligonucleotide(s) as given above are preferred concentrations for in vitro or ex vivo uses. The skilled person will understand that depending on the oligonucleotide(s) used, the target cell to be treated, the gene target and its expression levels, the medium used and the transfection and incubation conditions, the concentration of oligonucleotide(s) used may further vary and may need to be optimized any further.

More preferably, a compound preferably an oligonucleotide to be used in the invention to prevent, treat DMD or BMD are synthetically produced and administered directly to a cell, a tissue, an organ and/or patients in formulated form in a pharmaceutically acceptable composition or preparation. The delivery of a pharmaceutical composition to the subject is preferably carried out by one or more parenteral injections, e.g., intravenous and/or subcutaneous and/or intramuscular and/or intrathecal and/or intraventricular administrations, preferably injections, at one or at multiple sites in the human body.

A preferred oligonucleotide as defined herein optionally comprising one or more nucleotide analogs or equivalents of the invention modulates splicing in one or more muscle cells, including heart muscle cells, upon systemic delivery. In this respect, systemic delivery of an oligonucleotide comprising a specific nucleotide analog or equivalent might result in targeting a subset of muscle cells, while an oligonucleotide comprising a distinct nucleotide analog or equivalent might result in targeting of a different subset of muscle cells.

In this respect, systemic delivery of an oligonucleotide comprising a specific nucleotide analog or equivalent might result in targeting a subset of muscle cells, while an oligonucleotide comprising a distinct nucleotide analog or equivalent might result in targeting a different subset of muscle cells. Therefore, in this embodiment, it is preferred to use a combination of oligonucleotides comprising different nucleotide analogs or equivalents for modulating splicing of the DMD mRNA in at least one type of muscle cells.

In a preferred embodiment, there is provided a molecule or a viral-based vector for use as a medicament, preferably for modulating splicing of the DMD pre-mRNA, more preferably for promoting or inducing skipping of any of exon 43, 46, 50-53 as identified herein.

### Use

In yet a further aspect, the invention provides the use of an antisense oligonucleotide or molecule according to the invention, and/or a viral-based vector that expresses one or more antisense sequences according to the invention and/or a pharmaceutical composition, for modulating splicing of the DMD pre-mRNA. The splicing is preferably modulated in a human myogenic cell or muscle cell in vitro. More preferred is that splicing is modulated in a human muscle cell in vivo. Accordingly, the invention further relates to the use of the molecule as defined herein and/or the vector as defined herein and/or or the pharmaceutical composition as defined herein for modulating splicing of the DMD pre-mRNA or for the preparation of a medicament for the treatment of a DMD or BMD patient.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a molecule or a viral-based vector or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. Each embodiment as identified herein may be combined together unless otherwise indicated. All patent and literature references cited in the present specification are hereby incorporated by reference in their entirety.

The following examples are offered for illustrative purposes only, and are not intended to limit the scope of the present invention in any way.

### EXAMPLES

### Examples 1-4

### Materials and Methods

AON design was based on (partly) overlapping open secondary structures of the target exon RNA as predicted by them-fold program, on (partly) overlapping putative SR-protein binding sites as predicted by the ESE-finder software. AONs were synthesized by Prosensa Therapeutics B.V. (Leiden, Netherlands), and contain 2′-O-methyl RNA and full-length phosphorothioate (PS) backbones.

### Tissue Culturing, Transfection and RT-PCR Analysis

Myotube cultures derived from a healthy individual (“human control”) (examples 1, 3, and 4; exon 43, 50, 52 skipping) or a DMD patient carrying an exon 45 deletion (example 2; exon 46 skipping) were processed as described previously (Aartsma-Rus et al., Neuromuscul. Disord. 2002; 12: S71-77 and Hum Mol Genet 2003; 12(8): 907-14). For the screening of AONs, myotube cultures were transfected with 50 nM and 150 nM (example 2), 200 nM and 500 nM (example 4) or 500 nM only (examples 1 and 3) of each AON. Transfection reagent UNIFectylin (Prosensa Therapeutics BV, Netherlands) was used, with 2 μl UNIFectylin per μg AON. Exon skipping efficiencies were determined by nested RT-PCR analysis using primers in the exons flanking the targeted exons (43, 46, 50, 51, 52, or 53). PCR fragments were isolated from agarose gels for sequence verification. For quantification, the PCR products were analyzed using the DNA 1000 LabChips Kit on the Agilent 2100 bioanalyzer (Agilent Technologies, USA).

### Results

### DMD Exon 43 Skipping.

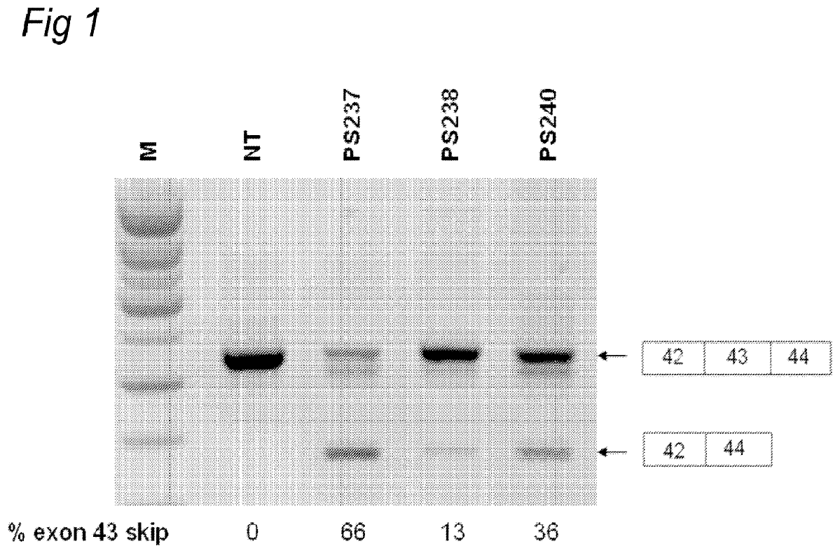

A series of AONs targeting sequences within exon 43 were designed and transfected in healthy control myotube cultures. Subsequent RT-PCR and sequence analysis of isolated RNA demonstrated that almost all AONs targeting a continuous nucleotide stretch within exon 43 herein defined as SEQ ID NO 2, was indeed capable of inducing exon 43 skipping. PS237 (SEQ ID NO: 65) reproducibly induced highest levels of exon 43 skipping (up to 66%) at 500 nM, as shown in FIG. 1. For comparison, also PS238 and PS240 are shown, inducing exon 43 skipping levels up to 13% and 36% respectively (FIG. 1). The precise skipping of exon 43 was confirmed by sequence analysis of the novel smaller transcript fragments. No exon 43 skipping was observed in non-treated cells (NT).

### DMD Exon 46 Skipping.

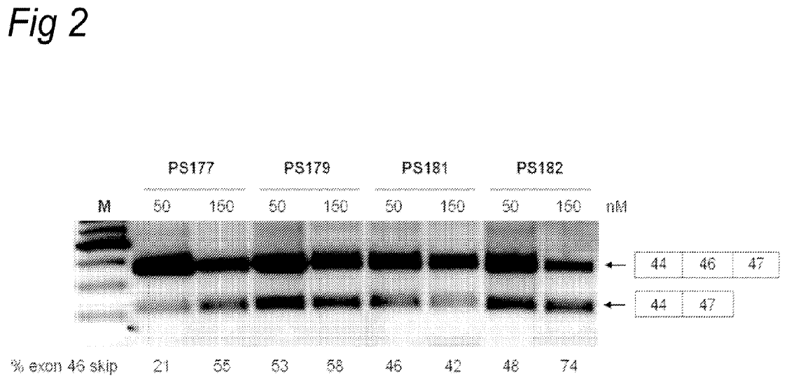

A series of AONs targeting sequences within exon 46 were designed and transfected in myotube cultures derived from a DMD patient carrying an exon 45 deletion in the DMD gene. For patients with such mutation antisense-induced exon 46 skipping would induce the synthesis of a novel, BMD-like dystrophin protein that may indeed alleviate one or more symptoms of the disease. Subsequent RT-PCR and sequence analysis of isolated RNA demonstrated that almost all AONs targeting a continuous nucleotide stretch within exon 46 herein defined as SEQ ID NO 3, was indeed capable of inducing exon 46 skipping, even at relatively low AON concentrations of 50 nM. PS182 (SEQ ID NO: 117) reproducibly induced highest levels of exon 46 skipping (up to 50% at 50 nM and 74% at 150 nM), as shown in FIG. 2. For comparison, also PS177, PS179, and PS181 are shown, inducing exon 46 skipping levels up to 55%, 58% and 42% respectively at 150 nM (FIG. 2). The precise skipping of exon 46 was confirmed by sequence analysis of the novel smaller transcript fragments. No exon 46 skipping was observed in non-treated cells (NT).

### DMD Exon 50 Skipping.

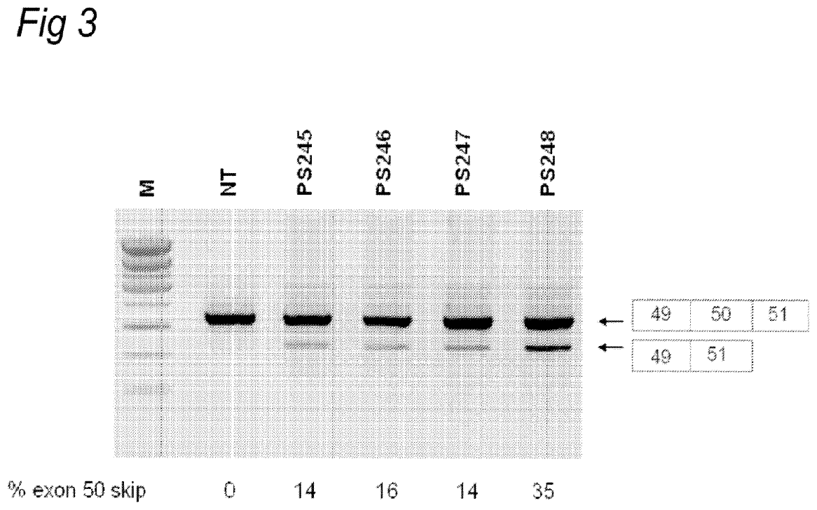

A series of AONs targeting sequences within exon 50 were designed and transfected in healthy control myotube cultures. Subsequent RT-PCR and sequence analysis of isolated RNA demonstrated that almost all AONs targeting a continuous nucleotide stretch within exon 50 herein defined as SEQ ID NO 4, was indeed capable of inducing exon 50 skipping. PS248 (SEQ ID NO: 127) reproducibly induced highest levels of exon 50 skipping (up to 35% at 500 nM), as shown in FIG. 3. For comparison, also PS245, PS246, and PS247 are shown, inducing exon 50 skipping levels up to 14-16% at 500 nM (FIG. 3). The precise skipping of exon 50 was confirmed by sequence analysis of the novel smaller transcript fragments. No exon 50 skipping was observed in non-treated cells (NT).

### DMD Exon 51 Skipping

A series of AONs targeting sequences within exon 51 were designed and transfected in healthy control myotube cultures. Subsequent RT-PCR and sequence analysis of isolated RNA demonstrated that almost all AONs targeting a continuous nucleotide stretch within exon 51 herein defined as SEQ ID NO 5, was indeed capable of inducing exon 51 skipping. The AON with SEQ ID NO 180 reproducibly induced highest levels of exon 51 skipping (not shown).

### DMD Exon 52 Skipping.

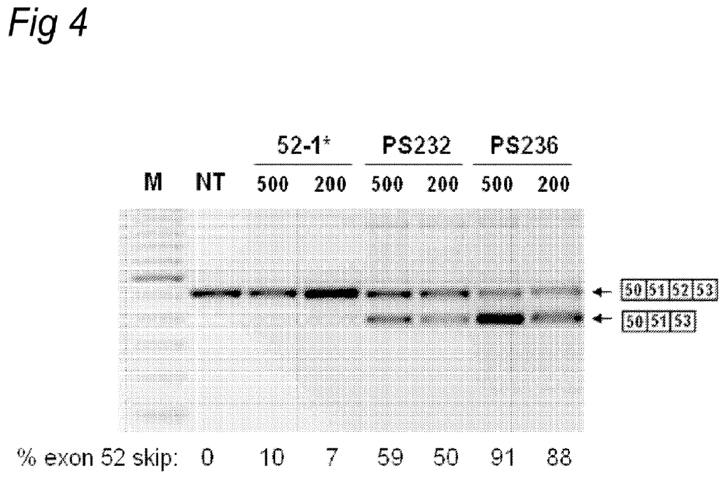

A series of AONs targeting sequences within exon 52 were designed and transfected in healthy control myotube cultures. Subsequent RT-PCR and sequence analysis of isolated RNA demonstrated that almost all AONs targeting a continuous nucleotide stretch within exon 52 herein defined as SEQ ID NO 6, was indeed capable of inducing exon 52 skipping. PS236 (SEQ ID NO: 299) reproducibly induced highest levels of exon 52 skipping (up to 88% at 200 nM and 91% at 500 nM), as shown in FIG. 4. For comparison, also PS232 and AON 52-1 (previously published by Aartsma-Rus et al. Oligonucleotides 2005) are shown, inducing exon 52 skipping at levels up to 59% and 10% respectively when applied at 500 nM (FIG. 4). The precise skipping of exon 52 was confirmed by sequence analysis of the novel smaller transcript fragments. No exon 52 skipping was observed in non-treated cells (NT).

### DMD Exon 53 Skipping

A series of AONs targeting sequences within exon 53 were designed and transfected in healthy control myotube cultures. Subsequent RT-PCR and sequence analysis of isolated RNA demonstrated that almost all AONs targeting a continuous nucleotide stretch within exon 53 herein defined as SEQ ID NO 7, was indeed capable of inducing exon 53 skipping. The AON with SEQ ID NO 328 reproducibly induced highest levels of exon 53 skipping (not shown).

SEQUENCE LISTING:

DMD GENE AMINO ACID SEQUENCE
SEQ ID NO 1:
MLWWEEVEDCYEREDVQKKTFTKWVNAQFSKFGKQHIENLFSDLQDGRRLLDLLEGL
TGQKLPKEKGSTRVHALNNVNKALRVLQNNNVDLVNIGSTDIVDGNHKLTLGLIWNIIL
HWQVKNVMKNIMAGLQQTNSEKILLSWVRQSTRNYPQVNVINFTTSWSDGLALNALIH
SHRPDLFDWNSVVCQQSATQRLEHAFNIARYQLGIEKLLDPEDVDTTYPDKKSILMYITS
LFQVLPQQVSIEAIQEVEMLPRPPKVTKEEHFQLHHQMHYSQQITVSLAQGYERTSSPKP
RFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGK
LSEDEETEVQEQMNLLNSRWECLRVASMEKQSNLHRVLMDLQNQKLKELNDWLTKTE
ERTRKMEEEPLGPDLEDLKRQVQQHKVLQEDLEQEQVRVNSLTHMVVVVDESSGDHA
TAALEEQLKVLGDRWANICRWTEDRWVLLQDILLKWQRLTEEQCLFSAWLSEKEDAV
NKIHTTGFKDQNEMLSSLQKLAVLKADLEKKKQSMGKLYSLKQDLLSTLKNKSVTQKT
EAWLDNFARCWDNLVQKLEKSTAQISQAVTTTQPSLTQTTVMETVTTVTTREQILVKH
AQEELPPPPPQKKRQITVDSEIRKRLDVDITELHSWITRSEAVLQSPEFAIFRKEGNFSDLK
EKVNAIEREKAEKFRKLQDASRSAQALVEQMVNEGVNADSIKQASEQLNSRWIEFCQLL
SERLNWLEYQNNIIAFYNQLQQLEQMTTTAENWLKIQPTTPSEPTAIKSQLKICKDEVNR
LSGLQPQIERLKIQSIALKEKGQGPMFLDADFVAFTNHFKQVFSDVQAREKELQTIFDTL
PPMRYQETMSAIRTWVQQSETKLSIPQLSVTDYEIMEQRLGELQALQSSLQEQQSGLYY
LSTTVKEMSKKAPSEISRKYQSEFEEIEGRWKKLSSQLVEHCQKLEEQMNKLRKIQNHIQ
TLKKWMAEVDVFLKEEWPALGDSEILKKQLKQCRLLVSDIQTIQPSLNSVNEGGQKIKN
EAEPEFASRLETELKELNTQWDHMCQQVYARKEALKGGLEKTVSLQKDLSEMHEWMT
QAEEEYLERDFEYKTPDELQKAVEEMKRAKEEAQQKEAKVKLLTESVNSVIAQAPPVA
QEALKKELETLTTNYQWLCTRLNGKCKTLEEVWACWHELLSYLEKANKWLNEVEFKL
KTTENIPGGAEEISEVLDSLENLMRHSEDNPNQIRILAQTLTDGGVMDELINEELETFNSR
WRELHEEAVRRQKLLEQSIQSAQETEKSLHLIQESLTFIDKQLAAYIADKVDAAQMPQE
AQKIQSDLTSHEISLEEMKKHNQGKEAAQRVLSQIDVAQKKLQDVSMKFRLFQKPANFE
QRLQESKMILDEVKMHLPALETKSVEQEVVQSQLNHCVNLYKSLSEVKSEVEMVIKTG
RQIVQKKQTENPKELDERVTALKLHYNELGAKVTERKQQLEKCLKLSRKMRKEMNVL
TEWLAATDMELTKRSAVEGMPSNLDSEVAWGKATQKEIEKQKVHLKSITEVGEALKTV
LGKKETLVEDKLSLLNSNWIAVTSRAEEWLNLLLEYQKHMETFDQNVDHITKWIIQADT
LLDESEKKKPQQKEDVLKRLKAELNDIRPKVDSTRDQAANLMANRGDHCRKLVEPQIS
ELNHRFAAISHRIKTGKASIPLKELEQFNSDIQKLLEPLEAEIQQGVNLKEEDFNKDMNED
NEGTVKELLQRGDNLQQRITDERKREEIKIKQQLLQTKHNALKDLRSQRRKKALEISHQ
WYQYKRQADDLLKCLDDIEKKLASLPEPRDERKIKEIDRELQKKKEELNAVRRQAEGLS
EDGAAMAVEPTQIQLSKRWREIESKFAQFRRLNFAQIHTVREETMMVMTEDMPLEISYV
PSTYLTEITHVSQALLEVEQLLNAPDLCAKDFEDLFKQEESLKNIKDSLQQSSGRIDIIHSK
KTAALQSATPVERVKLQEALSQLDFQWEKVNKMYKDRQGRFDRSVEKWRRFHYDIKIF
NQWLTEAEQFLRKTQIPENWEHAKYKWYLKELQDGIGQRQTVVRTLNATGEEIIQQSSK
TDASILQEKLGSLNLRWQEVCKQLSDRKKRLEEQKNILSEFQRDLNEFVLWLEEADNIA
SIPLEPGKEQQLKEKLEQVKLLVEELPLRQGILKQLNETGGPVLVSAPISPEEQDKLENKL
KQTNLQWIKVSRALPEKQGEIEAQIKDLGQLEKKLEDLEEQLNHLLLWLSPIRNQLEIYN
QPNQEGPFDVQETEIAVQAKQPDVEEILSKGQHLYKEKPATQPVKRKLEDLSSEWKAVN
RLLQELRAKQPDLAPGLTTIGASPTQTVTLVTQPVVTKETAISKLEMPSSLMLEVPALAD
FNRAWTELTDWLSLLDQVIKSQRVMVGDLEDINEMIIKQKATMQDLEQRRPQLEELITA
AQNLKNKTSNQEARTIITDRIERIQNQWDEVQEHLQNRRQQLNEMLKDSTQWLEAKEE
AEQVLGQARAKLESWKEGPYTVDAIQKKITETKQLAKDLRQWQTNVDVANDLALKLL
RDYSADDTRKVHMITENINASWRSIHKRVSEREAALEETHRLLQQFPLDLEKFLAWLTE
AETTANVLQDATRKERLLEDSKGVKELMKQWQDLQGEIEAHTDVYHNLDENSQKILRS
LEGSDDAVLLQRRLDNMNFKWSELRKKSLNIRSHLEASSDQWKRLHLSLQELLVWLQL
KDDELSRQAPIGGDFPAVQKQNDVHRAFKRELKTKEPVIMSTLETVRIFLTEQPLEGLEK
LYQEPRELPPEERAQNVTRLLRKQAEEVNTEWEKLNLHSADWQRKIDETLERLQELQEA
TDELDLKLRQAEVIKGSWQPVGDLLIDSLQDHLEKVKALRGEIAPLKENVSHVNDLARQ
LTTLGIQLSPYNLSTLEDLNTRWKLLQVAVEDRVRQLHEAHRDFGPASQHELSTSVQGP
WERAISPNKVPYYINHETQTTCWDHPKMTELYQSLADLNNVRFSAYRTAMKLRRLQKA
LCLDLLSLSAACDALDQHNLKQNDQPMDILQIINCLTTIYDRLEQEHNNLVNVPLCVDM
CLNWLLNVYDTGRTGRIRVLSFKTGIISLCKAHLEDKYRYLFKQVASSTGFCDQRRLGL
LLHDSIQIPRQLGEVASFGGSNIEPSVRSCFQFANNKPEIEAALFLDWMRLEPQSMVWLP
VLHRVAAAETAKHQAKCNICKECPIIGFRYRSLKHFNYDICQSCFFSGRVAKGHKMHYP
MVEYCTPTTSGEDVRDFAKVLKNKFRTKRYFAKHPRMGYLPVQTVLEGDNMETPVTLI
NFWPVDSAPASSPQLSHDDTHSRIEHYASRLAEMENSNGSYLNDSISPNESIDDEHLLIQH
YCQSLNQDSPLSQPRSPAQILISLESEERGELERILADLEEENRNLQAEYDRLKQQHEHKG
LSPLPSPPEMMPTSPQSPRDAELIAEAKLLRQHKGRLEARMQILEDHNKQLESQLHRLRQ
LLEQPQAEAKVNGTTVSSPSTSLQRSDSSQPMLLRVVGSQTSDSMGEEDLLSPPQDTSTG
LEEVMEQLNNSFPSSRGRNTPGKPMREDTM
SEQ ID NO 2 (EXON 43):
AGAUAGUCUACAACAAAGCUCAGGUCGGAUUGACAUUAUUCAUAGCAAGAAGAC
AG CAGCAUUGCAMGUGCAACGCCUGUGG
SEQ ID NO 3 (EXON 46):
UUAUGGUUGGAGGAAGCAGAUAACAUUGCUAGUAUCCCACUUGAACCUGGAMAG
AGCAGCAACUAAAAGAMAGC
SEQ ID NO 4 (EXON 50):
GGCGGUAMCCGUUUACUUCAAGAGCUGAGGGCAAAGCAGCCUGACCUAGCUCCU
GGACUGACCACUAUUGG
SEQ ID NO 5 (EXON 51):
CUCCUACUCAGACUGUUACUCUGGUGACACAACCUGUGGUUACUAAGGAMCUGC
CAUCUCCAAACUAGAAAUGCCAUCUUCCUUGAUGUUGGAGGUAC
SEQ ID NO 6 (EXON 52):
AUGCAGGAUUUGGAACAGAGGCGUCCCCAGUUGGAAGAACUCAUUACCGCUGCCC
AAAAUUU GAAAAACAAGACCAGCAAUCAAGAGGCU
SEQ ID NO 7 (EXON 53):
AAAUGUUAAAGGAUUCAACACAAUGGCUGGAAGCUAAGGAAGAAGCUGAGCAGG
UCUUAGGA CAGGCCAGAG

TABLE 1
OLIGONUCLEOTIDES FOR SKIPPING DMD GENE EXON 43

| | | | |
|---|---|---|---|
| SEQ ID | CCACAGGCGUUGCACUUUGCA | SEQ ID | UCUUCUUGCUAUGAAUAAUGUC |
| NO 8 | AUGC | NO 39 | AAU |
| SEQ ID | CACAGGCGUUGCACUUUGCAA | SEQ ID | CUUCUUGCUAUGAAUAAUGUCA |
| NO 9 | UGCU | NO 40 | AUC |
| SEQ ID | ACAGGCGUUGCACUUUGCAAU | SEQ ID | UUCUUGCUAUGAAUAAUGUCAA |
| NO 10 | GCUG | NO 41 | UCC |
| SEQ ID | CAGGCGUUGCACUUUGCAAUG | SEQ ID | UCUUGCUAUGAAUAAUGUCAAU |
| NO 11 | CUGC | NO 42 | CCG |
| SEQ ID | AGGCGUUGCACUUUGCAAUGC | SEQ ID | CUUGCUAUGAAUAAUGUCAAUC |
| NO 12 | UGCU | NO 43 | CGA |
| SEQ ID | GGCGUUGCACUUUGCAAUGCU | SEQ ID | UUGCUAUGAAUAAUGUCAAUCC |
| NO 13 | GCUG | NO 44 | GAC |
| SEQ ID | GCGUUGCACUUUGCAAUGCUG | SEQ ID | UGCUAUGAAUAAUGUCAAUCCG |
| NO 14 | CUGU | NO 45 | ACC |
| SEQ ID | CGUUGCACUUUGCAAUGCUGC | SEQ ID | GCUAUGAAUAAUGUCAAUCCGA |
| NO 15 | UGUC | NO 46 | CCU |
| SEQ ID | CGUUGCACUUUGCAAUGCUGC | SEQ ID | CUAUGAAUAAUGUCAAUCCGACC |
| NO 16 | UG | NO 47 | UG |
| PS240 | | | |
| SEQ ID | GUUGCACUUUGCAAUGCUGCU | SEQ ID | UAUGAAUAAUGUCAAUCCGACC |
| NO 17 | GUCU | NO 48 | UGA |
| SEQ ID | UUGCACUUUGCAAUGCUGCUG | SEQ ID | AUGAAUAAUGUCAAUCCGACCU |
| NO 18 | UCUU | NO 49 | GAG |
| SEQ ID | UGCACUUUGCAAUGCUGCUGU | SEQ ID | UGAAUAAUGUCAAUCCGACCUG |
| NO 19 | CUUC | NO 50 | AGC |
| SEQ ID | GCACUUUGCAAUGCUGCUGUC | SEQ ID | GAAUAAUGUCAAUCCGACCUGA |
| NO 20 | UUCU | NO 51 | GCU |
| SEQ ID | CACUUUGCAAUGCUGCUGUCU | SEQ ID | AAUAAUGUCAAUCCGACCUGAGC |
| NO 21 | UCUU | NO 52 | UU |
| SEQ ID | ACUUUGCAAUGCUGCUGUCUU | SEQ ID | AUAAUGUCAAUCCGACCUGAGCU |
| NO 22 | CUUG | NO 53 | UU |
| SEQ ID | CUUUGCAAUGCUGCUGUCUUC | SEQ ID | UAAUGUCAAUCCGACCUGAGCUU |
| NO 23 | UUGC | NO 54 | UG |
| SEQ ID | UUUGCAAUGCUGCUGUCUUCU | SEQ ID | AAUGUCAAUCCGACCUGAGCUUU |
| NO 24 | UGCU | NO 55 | GU |
| SEQ ID | UUGCAAUGCUGCUGUCUUCUU | SEQ ID | AUGUCAAUCCGACCUGAGCUUUG |
| NO 25 | GCUA | NO 56 | UU |
| SEQ ID | UGCAAUGCUGCUGUCUUCUUG | SEQ ID | UGUCAAUCCGACCUGAGCUUUGU |
| NO 26 | CUAU | NO 57 | UG |
| SEQ ID | GCAAUGCUGCUGUCUUCUUGC | SEQ ID | GUCAAUCCGACCUGAGCUUUGUU |
| NO 27 | UAUG | NO 58 | GU |
| SEQ ID | CAAUGCUGCUGUCUUCUUGCU | SEQ ID | UCAAUCCGACCUGAGCUUUGUUG |
| NO 28 | AUGA | NO 59 | UA |
| SEQ ID | AAUGCUGCUGUCUUCUUGCUA | SEQ ID | CAAUCCGACCUGAGCUUUGUUGU |
| NO 29 | UGAA | NO 60 | AG |
| SEQ ID | AUGCUGCUGUCUUCUUGCUAU | SEQ ID | AAUCCGACCUGAGCUUUGUUGU |
| NO 30 | GAAU | NO 61 | AGA |
| SEQ ID | UGCUGCUGUCUUCUUGCUAUG | SEQ ID | AUCCGACCUGAGCUUUGUUGUA |
| NO 31 | AAUA | NO 62 | GAC |
| SEQ ID | GCUGCUGUCUUCUUGCUAUGA | SEQ ID | UCCGACCUGAGCUUUGUUGUAG |
| NO 32 | AUAA | NO 63 | ACU |
| SEQ ID | CUGCUGUCUUCUUGCUAUGAA | SEQ ID | CCGACCUGAGCUUUGUUGUAGAC |
| NO 33 | UAAU | NO 64 | UA |
| SEQ ID | UGCUGUCUUCUUGCUAUGAAU | SEQ ID | CGACCUGAGCUUUGUUGUAG |
| NO 34 | AAUG | NO 65 | |
| | | PS237 | |
| SEQ ID | GCUGUCUUCUUGCUAUGAAUA | SEQ ID | CGACCUGAGCUUUGUUGUAGAC |
| NO 35 | AUGU | NO 66 | UAU |
| | | PS238 | |
| SEQ ID | CUGUCUUCUUGCUAUGAAUAA | SEQ ID | GACCUGAGCUUUGUUGUAGACU |
| NO 36 | UGUC | NO 67 | AUC |
| SEQ ID | UGUCUUCUUGCUAUGAAUAAU | SEQ ID | ACCUGAGCUUUGUUGUAGACUA |
| NO 37 | GUCA | NO 68 | UCA |
| SEQ ID | GUCUUCUUGCUAUGAAUAAUG | SEQ ID | CCUGA GCUUU GUUGU AGACU |
| NO 38 | UCAA | NO 69 | AUC |

TABLE 2
OLIGONUCLEOTIDES FOR SKIPPING DMD GENE EXON 46

| | | | |
|---|---|---|---|
| SEQ ID | GCUUUUCUUUUAGUUGCUGCUC | SEQ ID | CCAGGUUCAAGUGGGAUACUA |
| NO 70 | UUU | NO 97 | GCAA |
| PS179 | | | |
| SEQ ID | CUUUUCUUUUAGUUGCUGCUCU | SEQ ID | CAGGUUCAAGUGGGAUACUAG |
| NO 71 | UUU | NO 98 | CAAU |
| SEQ ID | UUUUCUUUUAGUUGCUGCUCU | SEQ ID | AGGUUCAAGUGGGAUACUAGC |
| NO 72 | UUUC | NO 99 | AAUG |
| SEQ ID | UUUCUUUUAGUUGCUGCUCUU | SEQ ID | GGUUCAAGUGGGAUACUAGCA |
| NO 73 | UUCC | NO 100 | AUGU |
| SEQ ID | UUCUUUUAGUUGCUGCUCUUU | SEQ ID | GUUCAAGUGGGAUACUAGCAA |
| NO 74 | UCCA | NO 101 | UGUU |
| SEQ ID | UCUUUUAGUUGCUGCUCUUUUC | SEQ ID | UUCAAGUGGGAUACUAGCAAU |
| NO 75 | CAG | NO 102 | GUUA |
| SEQ ID | CUUUUAGUUGCUGCUCUUUUCC | SEQ ID | UCAAGUGGGAUACUAGCAAUG |
| NO 76 | AGG | NO 103 | UUAU |
| SEQ ID | UUUUAGUUGCUGCUCUUUUCCA | SEQ ID | CAAGUGGGAUACUAGCAAUGU |
| NO 77 | GGU | NO 104 | UAUC |
| SEQ ID | UUUAGUUGCUGCUCUUUUCCAG | SEQ ID | AAGUGGGAUACUAGCAAUGUU |
| NO 78 | GUU | NO 105 | AUCU |
| SEQ ID | UUAGUUGCUGCUCUUUUCCAGG | SEQ ID | AGUGGGAUACUAGCAAUGUUA |
| NO 79 | UUC | NO 106 | UCUG |
| SEQ ID | UAGUUGCUGCUCUUUUCCAGGU | SEQ ID | GUGGGAUACUAGCAAUGUUAU |
| NO 80 | UCA | NO 107 | CUGC |
| SEQ ID | AGUUGCUGCUCUUUUCCAGGUU | SEQ ID | UGGGAUACUAGCAAUGUUAUC |
| NO 81 | CAA | NO 108 | UGCU |
| SEQ ID | GUUGCUGCUCUUUUCCAGGUUC | SEQ ID | GGGAUACUAGCAAUGUUAUCU |
| NO 82 | AAG | NO 109 | GCUU |
| SEQ ID | UUGCUGCUCUUUUCCAGGUUCA | SEQ ID | GGAUACUAGCAAUGUUAUCUG |
| NO 83 | AGU | NO 110 | CUUC |
| | | PS181 | |
| SEQ ID | UGCUGCUCUUUUCCAGGUUCAA | SEQ ID | GAUACUAGCAAUGUUAUCUGC |
| NO 84 | GUG | NO 111 | UUCC |
| SEQ ID | GCUGCUCUUUUCCAGGUUCAAG | SEQ ID | AUACUAGCAAUGUUAUCUGCU |
| NO 85 | UGG | NO 112 | UCCU |
| SEQ ID | CUGCUCUUUUCCAGGUUCAAGU | SEQ ID | UACUAGCAAUGUUAUCUGCUU |
| NO 86 | GGG | NO 113 | CCUC |
| SEQ ID | UGCUCUUUUCCAGGUUCAAGUG | SEQ ID | ACUAGCAAUGUUAUCUGCUUCC |
| NO 87 | GGA | NO 114 | UCC |
| SEQ ID | GCUCUUUUCCAGGUUCAAGUGG | SEQ ID | CUAGCAAUGUUAUCUGCUUCCU |
| NO 88 | GAC | NO 115 | CCA |
| SEQ ID | CUCUUUUCCAGGUUCAAGUGGG | SEQ ID | UAGCAAUGUUAUCUGCUUCCUC |
| NO 89 | AUA | NO 116 | CAA |
| SEQ ID | UCUUUUCCAGGUUCAAGUGGG | SEQ ID | AGCAAUGUUAUCUGCUUCCUCC |
| NO 90 | AUAC | NO 117 | AAC |
| | | PS182 | |
| SEQ ID | UCUUUUCCAGGUUCAAGUGG | SEQ ID | GCAAUGUUAUCUGCUUCCUCCA |
| NO 91 | | NO 118 | ACC |
| PS177 | | | |
| SEQ ID | CUUUUCCAGGUUCAAGUGGGA | SEQ ID | CAAUGUUAUCUGCUUCCUCCAA |
| NO 92 | UACU | NO 119 | CCA |
| SEQ ID | UUUUCCAGGUUCAAGUGGGAU | SEQ ID | AAUGUUAUCUGCUUCCUCCAAC |
| NO 93 | ACUA | NO 120 | CAU |
| SEQ ID | UUUCCAGGUUCAAGUGGGAUA | SEQ ID | AUGUUAUCUGCUUCCUCCAACC |
| NO 94 | CUAG | NO 121 | AUA |
| SEQ ID | UUCCAGGUUCAAGUGGGAUAC | SEQ ID | UGUUAUCUGCUUCCUCCAACCA |
| NO 95 | UAGC | NO 122 | UAA |
| SEQ ID | UCCAGGUUCAAGUGGGAUACU | | |
| NO 96 | AGCA | | |

TABLE 3
OLIGONUCLEOTIDES FOR SKIPPING DMD GENE EXON 50

| | | | |
|---|---|---|---|
| SEQ ID | CCAAUAGUGGUCAGUCCAGGA | SEQ ID | CUAGGUCAGGCUGCUUUGCCCU |
| NO 123 | GCUA | NO 146 | CAG |
| SEQ ID | CAAUAGUGGUCAGUCCAGGAG | SEQ ID | UAGGUCAGGCUGCUUUGCCCUC |
| NO 124 | CUAG | NO 147 | AGC |
| SEQ ID | AAUAGUGGUCAGUCCAGGAGC | SEQ ID | AGGUCAGGCUGCUUUGCCCUCA |
| NO 125 | UAGG | NO 148 | GCU |
| SEQ ID | AUAGUGGUCAGUCCAGGAGCU | SEQ ID | GGUCAGGCUGCUUUGCCCUCAG |
| NO 126 | AGGU | NO 149 | CUC |
| SEQ ID | AUAGUGGUCAGUCCAGGAGCU | SEQ ID | GUCAGGCUGCUUUGCCCUCAGC |
| NO 127 | | NO 150 | UCU |
| PS248 | | | |
| SEQ ID | UAGUGGUCAGUCCAGGAGCUA | SEQ ID | UCAGGCUGCUUUGCCCUCAGCU |
| NO 128 | GGUC | NO 151 | CUU |
| SEQ ID | AGUGGUCAGUCCAGGAGCUAG | SEQ ID | CAGGCUGCUUUGCCCUCAGCUC |
| NO 129 | GUCA | NO 152 | UUG |
| SEQ ID | GUGGUCAGUCCAGGAGCUAGG | SEQ ID | AGGCUGCUUUGCCCUCAGCUCU |
| NO 130 | UCAG | NO 153 | UGA |
| SEQ ID | UGGUCAGUCCAGGAGCUAGGU | SEQ ID | GGCUGCUUUGCCCUCAGCUCUU |
| NO 131 | CAGG | NO 154 | GAA |
| SEQ ID | GGUCAGUCCAGGAGCUAGGUC | SEQ ID | GCUGCUUUGCCCUCAGCUCUUG |
| NO 132 | AGGC | NO 155 | AAG |
| SEQ ID | GUCAGUCCAGGAGCUAGGUCA | SEQ ID | CUGCUUUGCCCUCAGCUCUUGA |
| NO 133 | GGCU | NO 156 | AGU |
| SEQ ID | UCAGUCCAGGAGCUAGGUCAG | SEQ ID | UGCUUUGCCCUCAGCUCUUGAA |
| NO 134 | GCUG | NO 157 | GUA |
| SEQ ID | CAGUCCAGGAGCUAGGUCAGG | SEQ ID | GCUUUGCCCUCAGCUCUUGAAG |
| NO 135 | CUGC | NO 158 | UAA |
| SEQ ID | AGUCCAGGAGCUAGGUCAGGC | SEQ ID | CUUUGCCCUCAGCUCUUGAAGU |
| NO 136 | UGCU | NO 159 | AAA |
| SEQ ID | GUCCAGGAGCUAGGUCAGGCU | SEQ ID | UUUGCCCUCAGCUCUUGAAGU |
| NO 137 | GCUU | NO 160 | AAAC |
| SEQ ID | UCCAGGAGCUAGGUCAGGCUG | SEQ ID | UUGCCCUCAGCUCUUGAAGUA |
| NO 138 | CUUU | NO 161 | AACG |
| SEQ ID | CCAGGAGCUAGGUCAGGCUGC | SEQ ID | UGCCCUCAGCUCUUGAAGUAA |
| NO 139 | UUUG | NO 162 | ACGG |
| SEQ ID | CAGGAGCUAGGUCAGGCUGCU | SEQ ID | GCCCUCAGCUCUUGAAGUAAAC |
| NO 140 | UUGC | NO 163 | GGU |
| SEQ ID | AGGAGCUAGGUCAGGCUGCUU | SEQ ID | CCCUCAGCUCUUGAAGUAAACG |
| NO 141 | UGCC | NO 164 | GUU |
| SEQ ID | GGAGCUAGGUCAGGCUGCUUU | SEQ ID | CCUCAGCUCUUGAAGUAAAC |
| NO 142 | GCCC | NO 165 | |
| | | PS246 | |
| SEQ ID | GAGCUAGGUCAGGCUGCUUUG | SEQ ID | CCUCAGCUCUUGAAGUAAACG |
| NO 143 | CCCU | NO 166 | |
| | | PS247 | |
| SEQ ID | AGCUAGGUCAGGCUGCUUUGC | SEQ ID | CUCAGCUCUUGAAGUAAACG |
| NO 144 | CCUC | NO 167 | |
| | | PS245 | |
| SEQ ID | GCUAGGUCAGGCUGCUUUGCC | SEQ ID | CCUCAGCUCUUGAAGUAAACG |
| NO 145 | CUCA | NO 529 | GUUU |
| SEQ ID | CUCAGCUCUUGAAGUAAACGG | SEQ ID | UCAGCUCUUGAAGUAAACGGU |
| NO 530 | UUUA | NO 531 | UUAC |
| SEQ ID | CAGCUCUUGAAGUAAACGGUU | SEQ ID | AGCUCUUGAAGUAAACGGUUU |
| NO 532 | UACC | NO 533 | ACCG |
| SEQ ID | GCUCUUGAAGUAAACGGUUUA | SEQ ID | CUCUUGAAGUAAACGGUUUAC |
| NO 534 | CCGC | NO 535 | CGCC |

TABLE 4
OLIGONUCLEOTIDES FOR SKIPPING DMD GENE EXON 51

| | | | |
|---|---|---|---|
| SEQ ID | GUACCUCCAACAUCAAGGAAGA | SEQ ID | GAGAUGGCAGUUUCCUUAGUAA |
| NO 168 | UGG | NO 205 | CCA |
| SEQ ID | UACCUCCAACAUCAAGGAAGAU | SEQ ID | AGAUGGCAGUUUCCUUAGUAAC |
| NO 169 | GGC | NO 206 | CAC |
| SEQ ID | ACCUCCAACAUCAAGGAAGAUG | SEQ ID | GAUGGCAGUUUCCUUAGUAACC |
| NO 170 | GCA | NO 207 | ACA |
| SEQ ID | CCUCCAACAUCAAGGAAGAUGG | SEQ ID | AUGGCAGUUUCCUUAGUAACCA |
| NO 171 | CAU | NO 208 | CAG |
| SEQ ID | CUCCAACAUCAAGGAAGAUGGC | SEQ ID | UGGCAGUUUCCUUAGUAACCAC |
| NO 172 | AUU | NO 209 | AGG |
| SEQ ID | UCCAACAUCAAGGAAGAUGGCA | SEQ ID | GGCAGUUUCCUUAGUAACCACA |
| NO 173 | UUU | NO 210 | GGU |
| SEQ ID | CCAACAUCAAGGAAGAUGGCAU | SEQ ID | GCAGUUUCCUUAGUAACCACAG |
| NO 174 | UUC | NO 211 | GUU |
| SEQ ID | CAACAUCAAGGAAGAUGGCAUU | SEQ ID | CAGUUUCCUUAGUAACCACAGG |
| NO 175 | UCU | NO 212 | UUG |
| SEQ ID | AACAUCAAGGAAGAUGGCAUUU | SEQ ID | AGUUUCCUUAGUAACCACAGGU |
| NO 176 | CUA | NO 213 | UGU |
| SEQ ID | ACAUCAAGGAAGAUGGCAUUUC | SEQ ID | GUUUCCUUAGUAACCACAGGUU |
| NO 177 | UAG | NO 214 | GUG |
| SEQ ID | CAUCAAGGAAGAUGGCAUUUCU | SEQ ID | UUUCCUUAGUAACCACAGGUUG |
| NO 178 | AGU | NO 215 | UGU |
| SEQ ID | AUCAAGGAAGAUGGCAUUUCUA | SEQ ID | UUCCUUAGUAACCACAGGUUGU |
| NO 179 | GUU | NO 216 | GUC |
| SEQ ID | UCAAGGAAGAUGGCAUUUCUAG | SEQ ID | UCCUUAGUAACCACAGGUUGUG |
| NO 180 | UUU | NO 217 | UCA |
| SEQ ID | CAAGGAAGAUGGCAUUUCUAGU | SEQ ID | CCUUAGUAACCACAGGUUGUGU |
| NO 181 | UUG | NO 218 | CAC |
| SEQ ID | AAGGAAGAUGGCAUUUCUAGUU | SEQ ID | CUUAGUAACCACAGGUUGUGUC |
| NO 182 | UGG | NO 219 | ACC |
| SEQ ID | AGGAAGAUGGCAUUUCUAGUUU | SEQ ID | UUAGUAACCACAGGUUGUGUCA |
| NO 183 | GGA | NO 220 | CCA |
| SEQ ID | GGAAGAUGGCAUUUCUAGUUUG | SEQ ID | UAGUAACCACAGGUUGUGUCAC |
| NO 184 | GAG | NO 221 | CAG |
| SEQ ID | GAAGAUGGCAUUUCUAGUUUGG | SEQ ID | AGUAACCACAGGUUGUGUCACC |
| NO 185 | AGA | NO 222 | AGA |
| SEQ ID | AAGAUGGCAUUUCUAGUUUGGA | SEQ ID | GUAACCACAGGUUGUGUCACCA |
| NO 186 | GAU | NO 223 | GAG |
| SEQ ID | AGAUGGCAUUUCUAGUUUGGAG | SEQ ID | UAACCACAGGUUGUGUCACCAG |
| NO 187 | AUG | NO 224 | AGU |
| SEQ ID | GAUGGCAUUUCUAGUUUGGAGA | SEQ ID | AACCACAGGUUGUGUCACCAGA |
| NO 188 | UGG | NO 225 | GUA |
| SEQ ID | AUGGCAUUUCUAGUUUGGAGAU | SEQ ID | ACCACAGGUUGUGUCACCAGAG |
| NO 189 | GGC | NO 226 | UAA |
| SEQ ID | UGGCAUUUCUAGUUUGGAGAUG | SEQ ID | CCACAGGUUGUGUCACCAGAGU |
| NO 190 | GCA | NO 227 | AAC |
| SEQ ID | GGCAUUUCUAGUUUGGAGAUGG | SEQ ID | CACAGGUUGUGUCACCAGAGUA |
| NO 191 | CAG | NO 228 | ACA |
| SEQ ID | GCAUUUCUAGUUUGGAGAUGGC | SEQ ID | ACAGGUUGUGUCACCAGAGUAA |
| NO 192 | AGU | NO 229 | CAG |
| SEQ ID | CAUUUCUAGUUUGGAGAUGGCA | SEQ ID | CAGGUUGUGUCACCAGAGUAAC |
| NO 193 | GUU | NO 230 | AGU |
| SEQ ID | AUUUCUAGUUUGGAGAUGGCAG | SEQ ID | AGGUUGUGUCACCAGAGUAACA |
| NO 194 | UUU | NO 231 | GUC |
| SEQ ID | UUUCUAGUUUGGAGAUGGCAGU | SEQ ID | GGUUGUGUCACCAGAGUAACAG |
| NO 195 | UUC | NO 232 | UCU |
| SEQ ID | UUCUAGUUUGGAGAUGGCAGUU | SEQ ID | GUUGUGUCACCAGAGUAACAGU |
| NO 196 | UCC | NO 233 | CUG |
| SEQ ID | UCUAGUUUGGAGAUGGCAGUUU | SEQ ID | UUGUGUCACCAGAGUAACAGUC |
| NO 197 | CCU | NO 234 | UGA |
| SEQ ID | CUAGUUUGGAGAUGGCAGUUUC | SEQ ID | UGUGUCACCAGAGUAACAGUCU |
| NO 198 | CUU | NO 235 | GAG |
| SEQ ID | UAGUUUGGAGAUGGCAGUUUCC | SEQ ID | GUGUCACCAGAGUAACAGUCUG |
| NO 199 | UUA | NO 236 | AGU |
| SEQ ID | AGUUUGGAGAUGGCAGUUUCCU | SEQ ID | UGUCACCAGAGUAACAGUCUGA |
| NO 200 | UAG | NO 237 | GUA |
| SEQ ID | GUUUGGAGAUGGCAGUUUCCUU | SEQ ID | GUCACCAGAGUAACAGUCUGAG |
| NO 201 | AGU | NO 238 | UAG |
| SEQ ID | UUUGGAGAUGGCAGUUUCCUUA | SEQ ID | UCACCAGAGUAACAGUCUGAGU |
| NO 202 | GUA | NO 239 | AGG |
| SEQ ID | UUGGAGAUGGCAGUUUCCUUAG | SEQ ID | CACCAGAGUAACAGUCUGAGUA |
| NO 203 | UAA | NO 240 | GGA |
| SEQ ID | UGGAGAUGGCAGUUUCCUUAGU | SEQ ID | ACCAGAGUAACAGUCUGAGUA |
| NO 204 | AAC | NO 241 | GGAG |

TABLE 5
OLIGONUCLEOTIDES FOR SKIPPING DMD GENE EXON 52

| | | | |
|---|---|---|---|
| SEQ ID | AGCCUCUUGAUUGCUGGUCUUG | SEQ ID | UUGGGCAGCGGUAAUGAGUUCU |
| NO 242 | UUU | NO 277 | UCC |
| SEQ ID | GCCUCUUGAUUGCUGGUCUUGU | SEQ ID | UGGGCAGCGGUAAUGAGUUCUU |
| NO 243 | UUU | NO 278 | CCA |
| SEQ ID | CCUCUUGAUUGCUGGUCUUGUU | SEQ ID | GGGCAGCGGUAAUGAGUUCUUC |
| NO 244 | UUU | NO 279 | CAA |
| SEQ ID | CCUCUUGAUUGCUGGUCUUG | SEQ ID | GGCAGCGGUAAUGAGUUCUUCC |
| NO 245 | | NO 280 | AAC |
| SEQ ID | CUCUUGAUUGCUGGUCUUGUU | SEQ ID | GCAGCGGUAAUGAGUUCUUCCA |
| NO 246 | UUUC | NO 281 | ACU |
| PS232 | | | |
| SEQ ID | UCUUGAUUGCUGGUCUUGUUU | SEQ ID | CAGCGGUAAUGAGUUCUUCCAA |
| NO 247 | UUCA | NO 282 | CUG |
| SEQ ID | CUUGAUUGCUGGUCUUGUUUU | SEQ ID | AGCGGUAAUGAGUUCUUCCAAC |
| NO 248 | UCAA | NO 283 | UGG |
| SEQ ID | UUGAUUGCUGGUCUUGUUUUU | SEQ ID | GCGGUAAUGAGUUCUUCCAACU |
| NO 249 | CAAA | NO 284 | GGG |
| SEQ ID | UGAUUGCUGGUCUUGUUUUUC | SEQ ID | CGGUAAUGAGUUCUUCCAACUG |
| NO 250 | AAAU | NO 285 | GGG |
| SEQ ID | GAUUGCUGGUCUUGUUUUUCA | SEQ ID | GGUAAUGAGUUCUUCCAACUGG |
| NO 251 | AAUU | NO 286 | GGA |
| SEQ ID | GAUUGCUGGUCUUGUUUUUC | SEQ ID | GGUAAUGAGUUCUUCCAACUGG |
| NO 252 | | NO 287 | |
| SEQ ID | AUUGCUGGUCUUGUUUUUCAA | SEQ ID | GUAAUGAGUUCUUCCAACUGGG |
| NO 253 | AUUU | NO 288 | GAC |
| SEQ ID | UUGCUGGUCUUGUUUUUCAAA | SEQ ID | UAAUGAGUUCUUCCAACUGGGG |
| NO 254 | UUUU | NO 289 | ACG |
| SEQ ID | UGCUGGUCUUGUUUUUCAAAU | SEQ ID | AAUGAGUUCUUCCAACUGGGGA |
| NO 255 | UUUG | NO 290 | CGC |
| SEQ ID | GCUGGUCUUGUUUUUCAAAUU | SEQ ID | AUGAGUUCUUCCAACUGGGGAC |
| NO 256 | UUGG | NO 291 | GCC |
| SEQ ID | CUGGUCUUGUUUUUCAAAUUU | SEQ ID | UGAGUUCUUCCAACUGGGGACG |
| NO 257 | UGGG | NO 292 | CCU |
| SEQ ID | UGGUCUUGUUUUUCAAAUUUU | SEQ ID | GAGUUCUUCCAACUGGGGACGC |
| NO 258 | GGGC | NO 293 | CUC |
| SEQ ID | GGUCUUGUUUUUCAAAUUUUG | SEQ ID | AGUUCUUCCAACUGGGGACGCC |
| NO 259 | GGCA | NO 294 | UCU |
| SEQ ID | GUCUUGUUUUUCAAAUUUUGG | SEQ ID | GUUCUUCCAACUGGGGACGCCU |
| NO 260 | GCAG | NO 295 | CUG |
| SEQ ID | UCUUGUUUUUCAAAUUUUGGG | SEQ ID | UUCUUCCAACUGGGGACGCCUC |
| NO 261 | CAGC | NO 296 | UGU |
| SEQ ID | CUUGUUUUUCAAAUUUUGGGC | SEQ ID | UCUUCCAACUGGGGACGCCUCU |
| NO 262 | AGCG | NO 297 | GUU |
| SEQ ID | UUGUUUUUCAAAUUUUGGGCA | SEQ ID | CUUCCAACUGGGGACGCCUCUG |
| NO 263 | GCGG | NO 298 | UUC |
| SEQ ID | UGUUUUUCAAAUUUUGGGCAG | SEQ ID | UUCCAACUGGGGACGCCUCUGU |
| NO 264 | CGGU | NO 299 | UCC |
| | | PS236 | |
| SEQ ID | GUUUUUCAAAUUUUGGGCAGC | SEQ ID | UCCAACUGGGGACGCCUCUGUU |
| NO 265 | GGUA | NO 300 | CCA |
| SEQ ID | UUUUUCAAAUUUUGGGCAGCG | SEQ ID | CCAACUGGGGACGCCUCUGUUC |
| NO 266 | GUAA | NO 301 | CAA |
| SEQ ID | UUUUCAAAUUUUGGGCAGCGG | SEQ ID | CAACUGGGGACGCCUCUGUUCC |
| NO 267 | UAAU | NO 302 | AAA |
| SEQ ID | UUUCAAAUUUUGGGCAGCGGU | SEQ ID | AACUGGGGACGCCUCUGUUCCA |
| NO 268 | AAUG | NO 303 | AAU |
| SEQ ID | UUCAAAUUUUGGGCAGCGGUA | SEQ ID | ACUGGGGACGCCUCUGUUCCAA |
| NO 269 | AUGA | NO 304 | AUC |
| SEQ ID | UCAAAUUUUGGGCAGCGGUAA | SEQ ID | CUGGGGACGCCUCUGUUCCAAA |
| NO 270 | UGAG | NO 305 | UCC |
| SEQ ID | CAAAUUUUGGGCAGCGGUAAU | SEQ ID | UGGGGACGCCUCUGUUCCAAAU |
| NO 271 | GAGU | NO 306 | CCU |
| SEQ ID | AAAUUUUGGGCAGCGGUAAUG | SEQ ID | GGGGACGCCUCUGUUCCAAAUC |
| NO 272 | AGUU | NO 307 | CUG |
| SEQ ID | AAUUUUGGGCAGCGGUAAUGA | SEQ ID | GGGACGCCUCUGUUCCAAAUCC |
| NO 273 | GUUC | NO 308 | UGC |
| SEQ ID | AUUUUGGGCAGCGGUAAUGAG | SEQ ID | GGACGCCUCUGUUCCAAAUCCU |
| NO 274 | UUCU | NO 309 | GCA |
| SEQ ID | UUUUGGGCAGCGGUAAUGAGU | SEQ ID | GACGCCUCUGUUCCAAAUCCUG |
| NO 275 | UCUU | NO 310 | CAU |
| SEQ ID | UUUGGGCAGCGGUAAUGAGUU | | |
| NO 276 | CUUC | | |

TABLE 6
OLIGONUCLEOTIDES FOR SKIPPING DMD GENE EXON 53

| | | | |
|---|---|---|---|
| SEQ ID | CUCUGGCCUGUCCUAAGACCU | SEQ ID | CAGCUUCUUCCUUAGCUUCCAG |
| NO 311 | GCUC | NO 335 | CCA |
| SEQ ID | UCUGGCCUGUCCUAAGACCUG | SEQ ID | AGCUUCUUCCUUAGCUUCCAGC |
| NO 312 | CUCA | NO 336 | CAU |
| SEQ ID | CUGGCCUGUCCUAAGACCUGC | SEQ ID | GCUUCUUCCUUAGCUUCCAGCC |
| NO 313 | UCAG | NO 337 | AUU |
| SEQ ID | UGGCCUGUCCUAAGACCUGCU | SEQ ID | CUUCUUCCUUAGCUUCCAGCCA |
| NO 314 | CAGC | NO 338 | UUG |
| SEQ ID | GGCCUGUCCUAAGACCUGCUC | SEQ ID | UUCUUCCUUAGCUUCCAGCCAU |
| NO 315 | AGCU | NO 339 | UGU |
| SEQ ID | GCCUGUCCUAAGACCUGCUCA | SEQ ID | UCUUCCUUAGCUUCCAGCCAUU |
| NO 316 | GCUU | NO 340 | GUG |
| SEQ ID | CCUGUCCUAAGACCUGCUCAG | SEQ ID | CUUCCUUAGCUUCCAGCCAUUG |
| NO 317 | CUUC | NO 341 | UGU |
| SEQ ID | CUGUCCUAAGACCUGCUCAGC | SEQ ID | UUCCUUAGCUUCCAGCCAUUGU |
| NO 318 | UUCU | NO 342 | GUU |
| SEQ ID | UGUCCUAAGACCUGCUCAGCU | SEQ ID | UCCUUAGCUUCCAGCCAUUGUG |
| NO 319 | UCUU | NO 343 | UUG |
| SEQ ID | GUCCUAAGACCUGCUCAGCUU | SEQ ID | CCUUAGCUUCCAGCCAUUGUGU |
| NO 320 | CUUC | NO 344 | UGA |
| SEQ ID | UCCUAAGACCUGCUCAGCUUC | SEQ ID | CUUAGCUUCCAGCCAUUGUGUU |
| NO 321 | UUCC | NO 345 | GAA |
| SEQ ID | CCUAAGACCUGCUCAGCUUCU | SEQ ID | UUAGCUUCCAGCCAUUGUGUUG |
| NO 322 | UCCU | NO 346 | AAU |
| SEQ ID | CUAAGACCUGCUCAGCUUCUU | SEQ ID | UAGCUUCCAGCCAUUGUGUUGA |
| NO 323 | CCUU | NO 347 | AUC |
| SEQ ID | UAAGACCUGCUCAGCUUCUUC | SEQ ID | AGCUUCCAGCCAUUGUGUUGAA |
| NO 324 | CUUA | NO 348 | UCC |
| SEQ ID | AAGACCUGCUCAGCUUCUUCC | SEQ ID | GCUUCCAGCCAUUGUGUUGAAU |
| NO 325 | UUAG | NO 349 | CCU |
| SEQ ID | AGACCUGCUCAGCUUCUUCCU | SEQ ID | CUUCCAGCCAUUGUGUUGAAUC |
| NO 326 | UAGC | NO 350 | CUU |
| SEQ ID | GACCUGCUCAGCUUCUUCCUU | SEQ ID | UUCCAGCCAUUGUGUUGAAUCC |
| NO 327 | AGCU | NO 351 | UUU |
| SEQ ID | ACCUGCUCAGCUUCUUCCUUA | SEQ ID | UCCAGCCAUUGUGUUGAAUCCU |
| NO 328 | GCUU | NO 352 | UUA |
| SEQ ID | CCUGCUCAGCUUCUUCCUUAG | SEQ ID | CCAGCCAUUGUGUUGAAUCCUU |
| NO 329 | CUUC | NO 353 | UAA |
| SEQ ID | CUGCUCAGCUUCUUCCUUAGC | SEQ ID | CAGCCAUUGUGUUGAAUCCUUU |
| NO 330 | UUCC | NO 354 | AAC |
| SEQ ID | UGCUCAGCUUCUUCCUUAGCU | SEQ ID | AGCCAUUGUGUUGAAUCCUUUA |
| NO 331 | UCCA | NO 355 | ACA |
| SEQ ID | GCUCAGCUUCUUCCUUAGCUU | SEQ ID | GCCAUUGUGUUGAAUCCUUUAA |
| NO 332 | CCAG | NO 356 | CAU |
| SEQ ID | CUCAGCUUCUUCCUUAGCUUC | SEQ ID | CCAUUGUGUUGAAUCCUUUAAC |
| NO 333 | CAGC | NO 357 | AUU |
| SEQ ID | UCAGCUUCUUCCUUAGCUUCC | SEQ ID | CAUUGUGUUGAAUCCUUUAACA |
| NO 334 | AGCC | NO 358 | UUU |

TABLE 7
OLIGONUCLEOTIDES FOR SKIPPING OTHER EXONS OF THE DMD
GENE AS IDENTIFIED
DMD Gene Exon 6

| | | | |
|---|---|---|---|
| SEQ ID | CAUUUUUGACCUACAUGUGG | SEQ ID | AUUUUUGACCUACAUGGGAAA |
| NO 359 | | NO 364 | G |
| SEQ ID | UUUGACCUACAUGUGGAAAG | SEQ ID | UACGAGUUGAUUGUCGGACCCA |
| NO 360 | | NO 365 | G |
| SEQ ID | UACAUUUUUGACCUACAUGUG | SEQ ID | GUGGUCUCCUUACCUAUGACUG |
| NO 361 | GAAA G | NO 366 | UGG |
| SEQ ID | GGUCUCCUUACCUAUGA | SEQ ID | UGUCUCAGUAAUCUUCUUACCU |
| NO 362 | | NO 367 | AU |
| SEQ ID | UCUUACCUAUGACUAUGGAUG | | |
| NO 363 | AGA | | |

DMD Gene Exon 7

| | | | |
|---|---|---|---|
| SEQ ID | UGCAUGUUCCAGUCGUUGUGU | SEQ ID | AUUUACCAACCUUCAGGAUCGA |
| NO 368 | GG | NO 370 | GUA |
| SEQ ID | CACUAUUCCAGUCAAAUAGGU | SEQ ID | GGCCUAAAACACAUACACAUA |
| NO 369 | CUGG | NO 371 | |

DMD Gene Exon 11

| | | | |
|---|---|---|---|
| SEQ ID | CCCUGAGGCAUUCCCAUCUUG | SEQ ID | CUUGAAUUUAGGAGAUUCAUCU |
| NO 372 | AAU | NO 374 | G |
| SEQ ID | AGGACUUACUUGCUUUGUUU | SEQ ID | CAUCUUCUGAUAAUUUUCCUGU |
| NO 373 | | NO 375 | U |

DMD Gene Exon 17

| | | | |
|---|---|---|---|
| SEQ ID | CCAUUACAGUUGUCUGUGUU | SEQ ID | UAAUCUGCCUCUUCUUUUGG |
| NO 376 | | NO 378 | |
| SEQ ID | UGACAGCCUGUGAAAUCUGUG | | |
| NO 377 | AG | | |

DMD Gene Exon 19

| | | | |
|---|---|---|---|
| SEQ ID | CAGCAGUAGUUGUCAUCUGC | SEQ ID | GCCUGAGCUGAUCUGCUGGCAU |
| NO 379 | | NO 381 | CUUGCA |
| | | | GUU |
| SEQ ID | GCCUGAGCUGAUCUGCUGGCA | SEQ ID | UCUGCUGGCAUCUUGC |
| NO 380 | UCUUGC | NO 382 | |

DMD Gene Exon 21

| | | | |
|---|---|---|---|
| SEQ ID | C | SEQ ID | CUGCAUCCAGGAACAUGGGUCC |
| NO 383 | GCCGGUUGACUUCAUCCUGUG | NO 386 | |
| SEQ ID | GUCUGCAUCCAGGAACAUGGG | SEQ ID | GUUGAAGAUCUGAUAGCCGGUU |
| NO 384 | UC | NO 387 | GA |
| SEQ ID | UACUUACUGUCUGUAGCUCUU | | |
| NO 385 | UCU | | |

DMD Gene Exon 44

| | | | |
|---|---|---|---|
| SEQ ID | UCAGCUUCUGUUAGCCACUG | SEQ ID | AGCUUCUGUUAGCCACUGAUUA |
| NO 388 | | NO 413 | AA |
| SEQ ID | UUCAGCUUCUGUUAGCCACU | SEQ ID | CAGCUUCUGUUAGCCACUGAUU |
| NO 389 | | NO 414 | AAA |
| SEQ ID | UUCAGCUUCUGUUAGCCACUG | SEQ ID | AGCUUCUGUUAGCCACUGAUUA |
| NO390 | | NO 415 | AA |
| SEQ ID | UCAGCUUCUGUUAGCCACUGA | SEQ ID | AGCUUCUGUUAGCCACUGAU |
| NO 391 | | NO 416 | |
| SEQ ID | UUCAGCUUCUGUUAGCCACUG | SEQ ID | GCUUCUGUUAGCCACUGAUU |
| NO 392 | A | NO 417 | |
| SEQ ID | UCAGCUUCUGUUAGCCACUGA | SEQ ID | AGCUUCUGUUAGCCACUGAUU |
| NO 393 | | NO 418 | |
| SEQ ID | UUCAGCUUCUGUUAGCCACUG | SEQ ID | GCUUCUGUUAGCCACUGAUUA |
| NO394 | A | NO 419 | |
| SEQ ID | UCAGCUUCUGUUAGCCACUGA | SEQ ID | AGCUUCUGUUAGCCACUGAUUA |
| NO 395 | U | NO 420 | |
| SEQ ID | UUCAGCUUCUGUUAGCCACUG | SEQ ID | GCUUCUGUUAGCCACUGAUUAA |
| NO 396 | AU | NO 421 | |
| SEQ ID | UCAGCUUCUGUUAGCCACUGA | SEQ ID | AGCUUCUGUUAGCCACUGAUUA |
| NO 397 | UU | NO 422 | A |
| SEQ ID | UUCAGCUUCUGUUAGCCACUG | SEQ ID | GCUUCUGUUAGCCACUGAUUAA |
| NO 398 | AUU | NO 423 | A |
| SEQ ID | UCAGCUUCUGUUAGCCACUGA | SEQ ID | AGCUUCUGUUAGCCACUGAUUA |
| NO 399 | UUA | NO 424 | AA |
| SEQ ID | UUCAGCUUCUGUUAGCCACUG | SEQ ID | GCUUCUGUUAGCCACUGAUUAA |
| NO 400 | AUA | NO 425 | A |
| SEQ ID | UCAGCUUCUGUUAGCCACUGA | SEQ ID | CCAUUUGUAUUUAGCAUGUUCC |
| NO 401 | UUAA | NO 426 | C |
| SEQ ID | UUCAGCUUCUGUUAGCCACUG | SEQ ID | AGAUACCAUUUGUAUUUAGC |
| NO 402 | AUUAA | NO 427 | |
| SEQ ID | UCAGCUUCUGUUAGCCACUGA | SEQ ID | GCCAUUUCUCAACAGAUCU |
| NO 403 | UUAAA | NO 428 | |
| SEQ ID | UUCAGCUUCUGUUAGCCACUG | SEQ ID | GCCAUUUCUCAACAGAUCUGUC |
| NO 404 | AUUAAA | NO 429 | A |
| SEQ ID | CAGCUUCUGUUAGCCACUG | SEQ ID | AUUCUCAGGAAUUUGUGUCUUU |
| NO 405 | | NO 430 | C |
| SEQ ID | CAGCUUCUGUUAGCCACUGAU | SEQ ID | UCUCAGGAAUUUGUGUCUUUC |
| NO 406 | | NO 431 | |
| SEQ ID | AGCUUCUGUUAGCCACUGAUU | SEQ ID | GUUCAGCUUCUGUUAGCC |
| NO 407 | | NO 432 | |
| SEQ ID | CAGCUUCUGUUAGCCACUGAU | SEQ ID | CUGAUUAAAUAUCUUUAUAUC |
| NO 408 | U | NO 433 | |
| SEQ ID | AGCUUCUGUUAGCCACUGAUU | SEQ ID | GCCGCCAUUUCUCAACAG |
| NO 409 | A | NO 434 | |
| SEQ ID | CAGCUUCUGUUAGCCACUGAU | SEQ ID | GUAUUUAGCAUGUUCCCA |
| NO 410 | UA | NO 435 | |
| SEQ ID | AGCUUCUGUUAGCCACUGAUU | SEQ ID | CAGGAAUUUGUGUCUUUC |
| NO 411 | AA | NO 436 | |
| SEQ ID | CAGCUUCUGUUAGCCACUGAU | | |
| NO 412 | UAA | | |

DMD Gene Exon 45

| | | | |
|---|---|---|---|
| SEQ ID | UUUGCCGCUGCCCAAUGCCAU | SEQ ID | GUUGCAUUCAAUGUUCUGACAA |
| NO 437 | CCUG | NO 470 | CAG |
| SEQ ID | AUUCAAUGUUCUGACAACAGU | SEQ ID | UUGCAUUCAAUGUUCUGACAAC |
| NO 438 | UUGC | NO 471 | AGU |
| SEQ ID | CCAGUUGCAUUCAAUGUUCUG | SEQ ID | UGCAUUCAAUGUUCUGACAACA |
| NO 439 | ACAA | NO 472 | GUU |
| SEQ ID | CAGUUGCAUUCAAUGUUCUGA | SEQ ID | GCAUUCAAUGUUCUGACAACAG |
| NO 440 | C | NO 473 | UUU |
| SEQ ID | AGUUGCAUUCAAUGUUCUGA | SEQ ID | CAUUCAAUGUUCUGACAACAGU |
| NO 441 | | NO 474 | UUG |
| SEQ ID | GAUUGCUGAAUUAUUUCUUCC | SEQ ID | AUUCAAUGUUCUGACAACAGUU |
| NO 442 | | NO 475 | UGC |
| SEQ ID | GAUUGCUGAAUUAUUUCUUCC | SEQ ID | UCAAUGUUCUGACAACAGUUUG |
| NO 443 | CCAG | NO 476 | CCG |
| SEQ ID | AUUGCUGAAUUAUUUCUUCCC | SEQ ID | CAAUGUUCUGACAACAGUUUGC |
| NO 444 | CAGU | NO 477 | CGC |
| SEQ ID | UUGCUGAAUUAUUUCUUCCCC | SEQ ID | AAUGUUCUGACAACAGUUUGCC |
| NO 445 | AGUU | NO 478 | GCU |
| SEQ ID | UGCUGAAUUAUUUCUUCCCCA | SEQ ID | AUGUUCUGACAACAGUUUGCCG |
| NO 446 | GUUG | NO 479 | CUG |
| SEQ ID | GCUGAAUUAUUUCUUCCCCAG | SEQ ID | UGUUCUGACAACAGUUUGCCGC |
| NO 447 | UUGC | NO 480 | UGC |
| SEQ ID | CUGAAUUAUUUCUUCCCCAGU | SEQ ID | GUUCUGACAACAGUUUGCCGCU |
| NO 448 | UGCA | NO 481 | GCC |
| SEQ ID | UGAAUUAUUUCUUCCCCAGUU | SEQ ID | UUCUGACAACAGUUUGCCGCUG |
| NO 449 | GCAU | NO 482 | CCC |
| SEQ ID | GAAUUAUUUCUUCCCCAGUUG | SEQ ID | UCUGACAACAGUUUGCCGCUGC |
| NO 450 | CAUU | NO 483 | CCA |
| SEQ ID | AAUUAUUUCUUCCCCAGUUGC | SEQ ID | CUGACAACAGUUUGCCGCUGCC |
| NO 451 | AUUC | NO 484 | CAA |
| SEQ ID | AUUAUUUCUUCCCCAGUUGCA | SEQ ID | UGACAACAGUUUGCCGCUGCCC |
| NO 452 | UUCA | NO 485 | AAU |
| SEQ ID | UUAUUUCUUCCCCAGUUGCAU | SEQ ID | GACAACAGUUUGCCGCUGCCCA |
| NO 453 | UCAA | NO 486 | AUG |
| SEQ ID | UAUUUCUUCCCCAGUUGCAUU | SEQ ID | ACAACAGUUUGCCGCUGCCCAA |
| NO 454 | CAAU | NO 487 | UGC |
| SEQ ID | AUUUCUUCCCCAGUUGCAUUC | SEQ ID | CAACAGUUUGCCGCUGCCCAAU |
| NO 455 | AAUG | NO 488 | GCC |
| SEQ ID | UUUCUUCCCCAGUUGCAUUCA | SEQ ID | AACAGUUUGCCGCUGCCCAAUG |
| NO 456 | AUGU | NO 489 | CCA |
| SEQ ID | UUCUUCCCCAGUUGCAUUCAA | SEQ ID | ACAGUUUGCCGCUGCCCAAUGC |
| NO 457 | UGUU | NO 490 | CAU |
| SEQ ID | UCUUCCCCAGUUGCAUUCAAU | SEQ ID | CAGUUUGCCGCUGCCCAAUGCC |
| NO 458 | GUUC | NO 491 | AUC |
| SEQ ID | CUUCCCCAGUUGCAUUCAAUG | SEQ ID | AGUUUGCCGCUGCCCAAUGCCA |
| NO 459 | UUCU | NO 492 | UCC |
| SEQ ID | UUCCCCAGUUGCAUUCAAUGU | SEQ ID | GUUUGCCGCUGCCCAAUGCCAU |
| NO 460 | UCUG | NO 493 | CCU |
| SEQ ID | UCCCCAGUUGCAUUCAAUGUU | SEQ ID | UUUGCCGCUGCCCAAUGCCAUC |
| NO 461 | CUGA | NO 494 | CUG |
| SEQ ID | CCCCAGUUGCAUUCAAUGUUC | SEQ ID | UUGCCGCUGCCCAAUGCCAUCC |
| NO 462 | UGAC | NO 495 | UGG |
| SEQ ID | CCCAGUUGCAUUCAAUGUUCU | SEQ ID | UGCCGCUGCCCAAUGCCAUCCU |
| NO 463 | GACA | NO 496 | GGA |
| SEQ ID | CCAGUUGCAUUCAAUGUUCUG | SEQ ID | GCCGCUGCCCAAUGCCAUCCUG |
| NO 464 | ACAA | NO 497 | GAG |
| SEQ ID | CAGUUGCAUUCAAUGUUCUGA | SEQ ID | CCGCUGCCCAAUGCCAUCCUGG |
| NO 465 | CAAC | NO 498 | AGU |
| SEQ ID | AGUUGCAUUCAAUGUUCUGAC | SEQ ID | CGCUGCCCAAUGCCAUCCUGGA |
| NO 466 | AACA | NO 499 | GUU |
| SEQ ID | UCC UGU AGA AUA CUG GCA | SEQ ID | UGUUUUUGAGGAUUGCUGAA |
| NO 467 | UC | NO 500 | |
| SEQ ID | UGCAGACCUCCUGCCACCGCAG | SEQ ID | UGUUCUGACAACAGUUUGCCGC |
| NO 468 | AUUCA | NO 501 | UGCCCA AUGCCAUCCUGG |
| SEQ ID | UUGCAGACCUCCUGCCACCGCA | | |
| NO 469 | GAUUC | | |
| | AGGCUUC | | |

DMD Gene Exon 55

| | | | |
|---|---|---|---|
| SEQ ID | CUGUUGCAGUAAUCUAUGAG | SEQ ID | UGCCAUUGUUUCAUCAGCUCUU |
| NO 502 | | NO 505 | U |
| SEQ ID | UGCAGUAAUCUAUGAGUUUC | SEQ ID | UCCUGUAGGACAUUGGCAGU |
| NO 503 | | NO 506 | |
| SEQ ID | GAGUCUUCUAGGAGCCUU | SEQ ID | CUUGGAGUCUUCUAGGAGCC |
| NO 504 | | NO 507 | |

DMD Gene Exon 57

| | | | |
|---|---|---|---|
| SEQ ID | UAGGUGCCUGCCGGCUU | SEQ ID | CUGAACUGCUGGAAAGUCGCC |
| NO 508 | | NO 510 | |
| SEQ ID | UUCAGCUGUAGCCACACC | SEQ ID | CUGGCUUCCAAAUGGGACCUGA |
| NO 509 | | NO 511 | AAAAGA |
| | | | AC |

DMD Gene Exon 59

| | | | |
|---|---|---|---|
| SEQ ID | CAAUUUUUCCCACUCAGUAUU | SEQ ID | UCCUCAGGAGGCAGCUCUAAAU |
| NO 512 | | NO 514 | |
| SEQ ID | UUGAAGUUCCUGGAGUCUU | | |
| NO 513 | | | |

DMD Gene Exon 62

| | | | |
|---|---|---|---|
| SEQ ID | UGGCUCUCUCCCAGGG | SEQ ID | GGGCACUUUGUUUGGCG |
| NO 515 | | NO 517 | |
| SEQ ID | GAGAUGGCUCUCUCCCAGGGA | | |
| NO 516 | CCCUGG | | |

DMD Gene Exon 63

| | | | |
|---|---|---|---|
| SEQ ID | GGUCCCAGCAAGUUGUUUG | SEQ ID | GUAGAGCUCUGUCAUUUUGGG |
| NO 518 | | NO 520 | |
| SEQ ID | UGGGAUGGUCCCAGCAAGUUG | | |
| NO 519 | UUUG | | |

DMD Gene Exon 65

| | | | |
|---|---|---|---|
| SEQ ID | GCUCAAGAGAUCCACUGCAAA | SEQ ID | UCUGCAGGAUAUCCAUGGGCUG |
| NO 521 | AAAC | NO 523 | GUC |
| SEQ ID | GCCAUACGUACGUAUCAUAAA | | |
| NO 522 | CAUUC | | |

DMD Gene Exon 66

| | |
|---|---|
| SEQ ID | GAUCCUCCCUGUUCGUCCCCUA |
| NO 524 | UUAUG |

DMD Gene Exon 69

| | |
|---|---|
| SEQ ID | UGCUUUAGACUCCUGUACCUG |
| NO 525 | AUA |

DMD Gene Exon 75

| | | | |
|---|---|---|---|
| SEQ ID | GGCGGCCUUUGUGUUGAC | SEQ ID | CCUUUAUGUUCGUGCUGCU |
| NO 526 | | NO 528 | |
| SEQ ID | GGACAGGCCUUUAUGUUCGUG | | |
| NO 527 | CUGC | | |

What is claimed is:

1. A molecule, which binds to a continuous stretch of at least 8 nucleotides within one of the following nucleotide sequences selected from:
(SEQ ID NO: 4)
5′-GGCGGTAAACCGUUUACUUCAAGAGCUGAGGGCAAAGCAGCCUGACCUA
GCUCCUGGACUGACCACUAUUGG-3′ for skipping of exon 50;
(SEQ ID NO: 2)
5′AGAUAGUCUACAACAAAGCUCAGGUCGGAUUGACAUUAUUCAUAGCAAG
AAGACAGCAGCAUUGCAAAGUGCAACGCCUGUGG-3′ for skipping of exon 43
(SEQ ID NO: 3)
5′UUAUGGUUGGAGGAAGCAGAUAACAUUGCUAGUAUCCCACUUGAACCUG
GAAAAGAGCAGCAACUAAAAGAAAAG C-3′ for skipping of exon 46;
(SEQ ID NO: 5)
5′CUCCUACUCAGACUGUUACUCUGGUGACACAACCUGUGGUUACUAAGGA
AACUGCCAUCUCCAAACUAGAAAUGC CAUCUUCCUUGAUG UUGGAGGUAC-3′
for skipping of exon 51;
(SEQ ID NO: 6)
5′AUGCAGGAUUUGGAACAGAGGCGUCCCCAGUUGGAAGAACUCAUUACCG
CUGCCCAAAAUUUGAAAAACAAGAC CAGCAAUCAAGAGGCU-3′ for
skipping of exon 52,
and
(SEQ ID NO: 7)
5′AAAUGUUAAAGGAUUCAACACAAUGGCUGGAAGCUAAGGAAAA
GCUGAGCAGGUCUUAGGACAGGCCAGAG-3′ for skipping of exon 53.

2. A molecule according to claim 1, wherein the molecule comprises or consists of the antisense nucleotide sequence selected from SEQ ID NO: 8-358, and/or SEQ ID NO 529-535 as depicted in tables 1 to 6.

3. A molecule according to claim 2, wherein the molecule comprises or consists of the antisense nucleotide sequence selected from SEQ ID NO:16, SEQ ID NO:65, SEQ ID NO:70, SEQ ID NO:91, SEQ ID NO:110, SEQ ID NO:117, SEQ ID NO:127, SEQ ID NO:165, SEQ ID NO:166, SEQ ID NO:167, SEQ ID NO:246, SEQ ID NO:299 and SEQ ID NO:357.

4. A molecule according to any one of claims 1 to 2, comprising a 2′-O-alkyl phosphorothioate antisense oligonucleotide.

5. A molecule according to claim 4, comprising a 2′-0 methyl phosphorothioate ribose.

6. A viral-based vector, comprising an expression cassette that drives expression of a molecule as defined in any one of claims 1-5.

7. A molecule according to any one of claims 1 to 5 or the viral-based vector according to claim 6 for use as a medicament, preferably for modulating splicing of the DMD pre-mRNA of a DMD or BMD patient or for the treatment of a DMD or BMD patient.

8. A pharmaceutical composition comprising a molecule as defined in any one of claims 1-5 and/or the vector of claim 6, a pharmaceutical acceptable carrier, and optionally combined with a molecule which is able to induce or promote skipping of at least one of exon 6, 7, 11, 17, 19, 21, 43, 44, 45, 50-53, 55, 57, 59, 62, 63, 65, 66, 69, or 75 of the DMD pre-mRNA of a patient.

9. A method for inducing and/or promoting skipping of at least one of exon 43, exon 46, exons 50-53 of the DMD pre-mRNA in a patient, preferably in an isolated cell of a patient, the method comprising providing said cell and/or said patient with a molecule as defined in any one of claims 1 to 5 or the vector of claim 6.

10. A method according to claim 9, wherein an additional molecule is used which is able to induce or promote skipping of at least one of exon 6, 7, 11, 17, 19, 21, 43, 44, 45, 50-53, 55, 57, 59, 62, 63, 65, 66, 69, or 75 of the DMD pre-mRNA of a patient.

11. Use of the molecule as defined in any one of claims 1-5, the vector of claim 6, or the pharmaceutical composition of claim 8 for modulating splicing of the DMD pre-mRNA or for the preparation of a medicament for the treatment of a DMD or BMD patient.


SEQUENCE LISTING (535 sequences)

SEQ ID NO: 1 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 2 - RNA, 83 nt, synthetic construct
  misc_feature 1..83: note = oligonucleotide
  source 1..83: mol_type = other RNA
agatagtctacaacaaagctcaggtcggattgacattattcatagcaagaagacagcagcattgcaaagtgcaacgcctgtgg

SEQ ID NO: 3 - RNA, 76 nt, synthetic construct
  misc_feature 1..76: note = oligonucleotide
  source 1..76: mol_type = other RNA
ttatggttggaggaagcagataacattgctagtatcccacttgaacctggaaaagagcagcaactaaaagaaaagc

SEQ ID NO: 4 - RNA, 72 nt, synthetic construct
  misc_feature 1..72: note = oligonucleotide
  source 1..72: mol_type = other RNA
ggcggtaaaccgtttacttcaagagctgagggcaaagcagcctgacctagctcctggactgaccactattgg

SEQ ID NO: 5 - RNA, 99 nt, synthetic construct
  misc_feature 1..99: note = oligonucleotide
  source 1..99: mol_type = other RNA
ctcctactcagactgttactctggtgacacaacctgtggttactaaggaaactgccatctccaaactagaaatgccatcttccttgatgttggaggtac

SEQ ID NO: 6 - RNA, 90 nt, synthetic construct
  misc_feature 1..90: note = oligonucleotide
  source 1..90: mol_type = other RNA
atgcaggatttggaacagaggcgtccccagttggaagaactcattaccgctgcccaaaatttgaaaaacaagaccagcaatcaagaggct

SEQ ID NO: 7 - RNA, 72 nt, synthetic construct
  misc_feature 1..72: note = oligonucleotide
  source 1..72: mol_type = other RNA
aaatgttaaaggattcaacacaatggctggaagctaaggaagaagctgagcaggtcttaggacaggccagag

SEQ ID NO: 8 - RNA, 25 nt, synthetic construct
  misc_feature 1..25: note = oligonucleotide
  source 1..25: mol_type = other RNA
ccacaggcgttgcactttgcaatgc

SEQ ID NO: 9 - RNA, 25 nt, synthetic construct
  misc_feature 1..25: note = oligonucleotide
  source 1..25: mol_type = other RNA
cacaggcgttgcactttgcaatgct

SEQ ID NO: 10 - RNA, 25 nt, synthetic construct
  misc_feature 1..25: note = oligonucleotide
  source 1..25: mol_type = other RNA
acaggcgttgcactttgcaatgctg

SEQ ID NO: 11 - RNA, 25 nt, synthetic construct
  misc_feature 1..25: note = oligonucleotide
  source 1..25: mol_type = other RNA
caggcgttgcactttgcaatgctgc

SEQ ID NO: 12 - RNA, 25 nt, synthetic construct
  misc_feature 1..25: note = oligonucleotide
  source 1..25: mol_type = other RNA
aggcgttgcactttgcaatgctgct

SEQ ID NO: 13 - RNA, 25 nt, synthetic construct
  misc_feature 1..25: note = oligonucleotide
  source 1..25: mol_type = other RNA
ggcgttgcactttgcaatgctgctg

SEQ ID NO: 14 - RNA, 25 nt, synthetic construct
  misc_feature 1..25: note = oligonucleotide
  source 1..25: mol_type = other RNA
gcgttgcactttgcaatgctgctgt

SEQ ID NO: 15 - RNA, 25 nt, synthetic construct
  misc_feature 1..25: note = oligonucleotide
  source 1..25: mol_type = other RNA
cgttgcactttgcaatgctgctgtc

SEQ ID NO: 16 - RNA, 23 nt, synthetic construct
  misc_feature 1..23: note = oligonucleotide
  source 1..23: mol_type = other RNA
cgttgcactttgcaatgctgctg

SEQ ID NO: 17 - RNA, 25 nt, synthetic construct
  misc_feature 1..25: note = oligonucleotide
  source 1..25: mol_type = other RNA
gttgcactttgcaatgctgctgtct

SEQ ID NO: 18 - RNA, 25 nt, synthetic construct
  misc_feature 1..25: note = oligonucleotide
  source 1..25: mol_type = other RNA
ttgcactttgcaatgctgctgtctt

SEQ ID NO: 19 - RNA, 25 nt, synthetic construct
  misc_feature 1..25: note = oligonucleotide
  source 1..25: mol_type = other RNA
tgcactttgcaatgctgctgtcttc

SEQ ID NO: 20 - RNA, 25 nt, synthetic construct
  misc_feature 1..25: note = oligonucleotide
  source 1..25: mol_type = other RNA
gcactttgcaatgctgctgtcttct

SEQ ID NO: 21 - RNA, 25 nt, synthetic construct
  misc_feature 1..25: note = oligonucleotide
  source 1..25: mol_type = other RNA
cactttgcaatgctgctgtcttctt

SEQ ID NO: 22 - RNA, 25 nt, synthetic construct
  misc_feature 1..25: note = oligonucleotide
  source 1..25: mol_type = other RNA
actttgcaatgctgctgtcttcttg

SEQ ID NO: 23 - RNA, 25 nt, synthetic construct
  misc_feature 1..25: note = oligonucleotide
  source 1..25: mol_type = other RNA
ctttgcaatgctgctgtcttcttgc

SEQ ID NO: 24 - RNA, 25 nt, synthetic construct
  misc_feature 1..25: note = oligonucleotide
  source 1..25: mol_type = other RNA
tttgcaatgctgctgtcttcttgct

SEQ ID NO: 25 - RNA, 25 nt, synthetic construct
  misc_feature 1..25: note = oligonucleotide
  source 1..25: mol_type = other RNA
ttgcaatgctgctgtcttcttgcta

SEQ ID NO: 26 - RNA, 25 nt, synthetic construct
  misc_feature 1..25: note = oligonucleotide
  source 1..25: mol_type = other RNA
tgcaatgctgctgtcttcttgctat

SEQ ID NO: 27 - RNA, 25 nt, synthetic construct
  misc_feature 1..25: note = oligonucleotide
  source 1..25: mol_type = other RNA
gcaatgctgctgtcttcttgctatg

SEQ ID NO: 28 - RNA, 25 nt, synthetic construct
  misc_feature 1..25: note = oligonucleotide
  source 1..25: mol_type = other RNA
caatgctgctgtcttcttgctatga

SEQ ID NO: 29 - RNA, 25 nt, synthetic construct
  misc_feature 1..25: note = olignucleotide
  source 1..25: mol_type = other RNA
aatgctgctgtcttcttgctatgaa

SEQ ID NO: 30 - RNA, 25 nt, synthetic construct
  misc_feature 1..25: note = oligonucleotide
  source 1..25: mol_type = other RNA
atgctgctgtcttcttgctatgaat

SEQ ID NO: 31 - RNA, 25 nt, synthetic construct
  misc_feature 1..25: note = oligonucleotide
  source 1..25: mol_type = other RNA
tgctgctgtcttcttgctatgaata

SEQ ID NO: 32 - RNA, 25 nt, synthetic construct
  misc_feature 1..25: note = oligonucleotide
  source 1..25: mol_type = other RNA
gctgctgtcttcttgctatgaataa

SEQ ID NO: 33 - RNA, 25 nt, synthetic construct
  misc_feature 1..25: note = oligonucleotide
  source 1..25: mol_type = other RNA
ctgctgtcttcttgctatgaataat

SEQ ID NO: 34 - RNA, 25 nt, synthetic construct
  misc_feature 1..25: note = oligonucleotide
  source 1..25: mol_type = other RNA
tgctgtcttcttgctatgaataatg

SEQ ID NO: 35 - RNA, 25 nt, synthetic construct
  misc_feature 1..25: note = oligonucleotide
  source 1..25: mol_type = other RNA
gctgtcttcttgctatgaataatgt

SEQ ID NO: 36 - RNA, 25 nt, synthetic construct
  misc_feature 1..25: note = oligonucleotide
  source 1..25: mol_type = other RNA
ctgtcttcttgctatgaataatgtc

SEQ ID NO: 37 - RNA, 25 nt, synthetic construct
  misc_feature 1..25: note = oligonucleotide
  source 1..25: mol_type = other RNA
tgtcttcttgctatgaataatgtca

SEQ ID NO: 38 - RNA, 25 nt, synthetic construct
  misc_feature 1..25: note = oligonucleotide
  source 1..25: mol_type = other RNA
gtcttcttgctatgaataatgtcaa

SEQ ID NO: 39 - RNA, 25 nt, synthetic construct
  misc_feature 1..25: note = oligonucleotide
  source 1..25: mol_type = other RNA
tcttcttgctatgaataatgtcaat

SEQ ID NO: 40 - RNA, 25 nt, synthetic construct
  misc_feature 1..25: note = oligonucleotide
  source 1..25: mol_type = other RNA
cttcttgctatgaataatgtcaatc

SEQ ID NO: 41 - RNA, 25 nt, synthetic construct
  misc_feature 1..25: note = oligonucleotide
  source 1..25: mol_type = other RNA
ttcttgctatgaataatgtcaatcc

SEQ ID NO: 42 - RNA, 25 nt, synthetic construct
  misc_feature 1..25: note = oligonucleotide
  source 1..25: mol_type = other RNA
tcttgctatgaataatgtcaatccg

SEQ ID NO: 43 - RNA, 25 nt, synthetic construct
  misc_feature 1..25: note = oligonucleotide
  source 1..25: mol_type = other RNA
cttgctatgaataatgtcaatccga

SEQ ID NO: 44 - RNA, 25 nt, synthetic construct
  misc_feature 1..25: note = oligonucleotide
  source 1..25: mol_type = other RNA
ttgctatgaataatgtcaatccgac

SEQ ID NO: 45 - RNA, 25 nt, synthetic construct
  misc_feature 1..25: note = oligonucleotide
  source 1..25: mol_type = other RNA
tgctatgaataatgtcaatccgacc

SEQ ID NO: 46 - RNA, 25 nt, synthetic construct
  misc_feature 1..25: note = oligonucleotide
  source 1..25: mol_type = other RNA
gctatgaataatgtcaatccgacct

SEQ ID NO: 47 - RNA, 25 nt, synthetic construct
  misc_feature 1..25: note = oligonucleotide
  source 1..25: mol_type = other RNA
ctatgaataatgtcaatccgacctg

SEQ ID NO: 48 - RNA, 25 nt, synthetic construct
  misc_feature 1..25: note = oligonucleotide
  source 1..25: mol_type = other RNA
tatgaataatgtcaatccgacctga

SEQ ID NO: 49 - RNA, 25 nt, synthetic construct
  misc_feature 1..25: note = oligonucleotide
  source 1..25: mol_type = other RNA
atgaataatgtcaatccgacctgag

SEQ ID NO: 50 - RNA, 25 nt, synthetic construct
  misc_feature 1..25: note = oligonucleotide
  source 1..25: mol_type = other RNA
tgaataatgtcaatccgacctgagc

SEQ ID NO: 51 - RNA, 25 nt, synthetic construct
  misc_feature 1..25: note = oligonucleotide
  source 1..25: mol_type = other RNA
gaataatgtcaatccgacctgagct

SEQ ID NO: 52 - RNA, 25 nt, synthetic construct
  misc_feature 1..25: note = oligonucleotide
  source 1..25: mol_type = other RNA
aataatgtcaatccgacctgagctt

SEQ ID NO: 53 - RNA, 25 nt, synthetic construct
  misc_feature 1..25: note = oligonucleotide
  source 1..25: mol_type = other RNA
ataatgtcaatccgacctgagcttt

SEQ ID NO: 54 - RNA, 25 nt, synthetic construct
  misc_feature 1..25: note = oligonucleotide
  source 1..25: mol_type = other RNA
taatgtcaatccgacctgagctttg

SEQ ID NO: 55 - RNA, 25 nt, synthetic construct
  misc_feature 1..25: note = oligonucleotide
  source 1..25: mol_type = other RNA
aatgtcaatccgacctgagctttgt

SEQ ID NO: 56 - RNA, 25 nt, synthetic construct
  misc_feature 1..25: note = oligonucleotide
  source 1..25: mol_type = other RNA
atgtcaatccgacctgagctttgtt

SEQ ID NO: 57 - RNA, 25 nt, synthetic construct
  misc_feature 1..25: note = oligonucleotide
  source 1..25: mol_type = other RNA
tgtcaatccgacctgagctttgttg

SEQ ID NO: 58 - RNA, 25 nt, synthetic construct
  misc_feature 1..25: note = oligonucleotide
  source 1..25: mol_type = other RNA
gtcaatccgacctgagctttgttgt

SEQ ID NO: 59 - RNA, 25 nt, synthetic construct
  misc_feature 1..25: note = oligonucleotide
  source 1..25: mol_type = other RNA
tcaatccgacctgagctttgttgta

SEQ ID NO: 60 - RNA, 25 nt, synthetic construct
  misc_feature 1..25: note = oligonucleotide
  source 1..25: mol_type = other RNA
caatccgacctgagctttgttgtag

SEQ ID NO: 61 - RNA, 25 nt, synthetic construct
  misc_feature 1..25: note = oligonucleotide
  source 1..25: mol_type = other RNA
aatccgacctgagctttgttgtaga

SEQ ID NO: 62 - RNA, 25 nt, synthetic construct
  misc_feature 1..25: note = olignucleotide
  source 1..25: mol_type = other RNA
atccgacctgagctttgttgtagac

SEQ ID NO: 63 - RNA, 25 nt, synthetic construct
  misc_feature 1..25: note = oligonucleotide
  source 1..25: mol_type = other RNA
tccgacctgagctttgttgtagact

SEQ ID NO: 64 - RNA, 25 nt, synthetic construct
  misc_feature 1..25: note = oligonucleotide
  source 1..25: mol_type = other RNA
ccgacctgagctttgttgtagacta

SEQ ID NO: 65 - RNA, 20 nt, synthetic construct
  misc_feature 1..20: note = oligonucleotide
  source 1..20: mol_type = other RNA
cgacctgagctttgttgtag

SEQ ID NO: 66 - RNA, 25 nt, synthetic construct
  misc_feature 1..25: note = oligonucleotide
  source 1..25: mol_type = other RNA
cgacctgagctttgttgtagactat

SEQ ID NO: 67 - RNA, 25 nt, synthetic construct
  misc_feature 1..25: note = oligonucleotide
  source 1..25: mol_type = other RNA
gacctgagctttgttgtagactatc

SEQ ID NO: 68 - RNA, 25 nt, synthetic construct
  misc_feature 1..25: note = oligonucleotide
  source 1..25: mol_type = other RNA
acctgagctttgttgtagactatca

SEQ ID NO: 69 - RNA, 23 nt, synthetic construct
  misc_feature 1..23: note = oligonucleotide
  source 1..23: mol_type = other RNA
cctgagctttgttgtagactatc

SEQ ID NO: 70 - RNA, 25 nt, synthetic construct
  misc_feature 1..25: note = oligonucleotide
  source 1..25: mol_type = other RNA
gcttttcttttagttgctgctcttt

SEQ ID NO: 71 - RNA, 25 nt, synthetic construct
  misc_feature 1..25: note = oligonucleotide
  source 1..25: mol_type = other RNA
cttttcttttagttgctgctctttt

SEQ ID NO: 72 - RNA, 25 nt, synthetic construct
  misc_feature 1..25: note = oligonucleotide
  source 1..25: mol_type = other RNA
ttttcttttagttgctgctcttttc

SEQ ID NO: 73 - RNA, 25 nt, synthetic construct
  misc_feature 1..25: note = oligonucleotide
  source 1..25: mol_type = other RNA
tttcttttagttgctgctcttttcc

SEQ ID NO: 74 - RNA, 25 nt, synthetic construct
  misc_feature 1..25: note = oligonucleotide
  source 1..25: mol_type = other RNA
ttcttttagttgctgctcttttcca

SEQ ID NO: 75 - RNA, 25 nt, synthetic construct
  misc_feature 1..25: note = oligonucleotide
  source 1..25: mol_type = other RNA
tcttttagttgctgctcttttccag

SEQ ID NO: 76 - RNA, 25 nt, synthetic construct
  misc_feature 1..25: note = oligonucleotide
  source 1..25: mol_type = other RNA
cttttagttgctgctcttttccagg

SEQ ID NO: 77 - RNA, 25 nt, synthetic construct
  misc_feature 1..25: note = oligonucleotide
  source 1..25: mol_type = other RNA
ttttagttgctgctcttttccaggt

SEQ ID NO: 78 - RNA, 25 nt, synthetic construct
  misc_feature 1..25: note = oligonucleotide
  source 1..25: mol_type = other RNA
tttagttgctgctcttttccaggtt

SEQ ID NO: 79 - RNA, 25 nt, synthetic construct
  misc_feature 1..25: note = oligonucleotide
  source 1..25: mol_type = other RNA
ttagttgctgctcttttccaggttc

SEQ ID NO: 80 - RNA, 25 nt, synthetic construct
  misc_feature 1..25: note = oligonucleotide
  source 1..25: mol_type = other RNA
tagttgctgctcttttccaggttca

SEQ ID NO: 81 - RNA, 25 nt, synthetic construct
  misc_feature 1..25: note = oligonucleotide
  source 1..25: mol_type = other RNA
agttgctgctcttttccaggttcaa

SEQ ID NO: 82 - RNA, 25 nt, synthetic construct
  misc_feature 1..25: note = oligonucleotide
  source 1..25: mol_type = other RNA
gttgctgctcttttccaggttcaag

SEQ ID NO: 83 - RNA, 25 nt, synthetic construct
  misc_feature 1..25: note = oligonucleotide
  source 1..25: mol_type = other RNA
ttgctgctcttttccaggttcaagt

SEQ ID NO: 84 - RNA, 25 nt, synthetic construct
  misc_feature 1..25: note = oligonucleotide
  source 1..25: mol_type = other RNA
tgctgctcttttccaggttcaagtg

SEQ ID NO: 85 - RNA, 25 nt, synthetic construct
  misc_feature 1..25: note = oligonucleotide
  source 1..25: mol_type = other RNA
gctgctcttttccaggttcaagtgg

SEQ ID NO: 86 - RNA, 25 nt, synthetic construct
  misc_feature 1..25: note = oligonucleotide
  source 1..25: mol_type = other RNA
ctgctcttttccaggttcaagtggg

SEQ ID NO: 87 - RNA, 25 nt, synthetic construct
  misc_feature 1..25: note = oligonucleotide
  source 1..25: mol_type = other RNA
tgctcttttccaggttcaagtggga

SEQ ID NO: 88 - RNA, 25 nt, synthetic construct
  misc_feature 1..25: note = oligonucleotide
  source 1..25: mol_type = other RNA
gctcttttccaggttcaagtgggac

SEQ ID NO: 89 - RNA, 25 nt, synthetic construct
  misc_feature 1..25: note = oligonucleotide
  source 1..25: mol_type = other RNA
ctcttttccaggttcaagtgggata

SEQ ID NO: 90 - RNA, 25 nt, synthetic construct
  misc_feature 1..25: note = oligonucleotide
  source 1..25: mol_type = other RNA
tcttttccaggttcaagtgggatac

SEQ ID NO: 91 - RNA, 20 nt, synthetic construct
  misc_feature 1..20: note = oligonucleotide
  source 1..20: mol_type = other RNA
tcttttccaggttcaagtgg

SEQ ID NO: 92 - RNA, 25 nt, synthetic construct
  misc_feature 1..25: note = oligonucleotide
  source 1..25: mol_type = other RNA
cttttccaggttcaagtgggatact

SEQ ID NO: 93 - RNA, 25 nt, synthetic construct
  misc_feature 1..25: note = oligonucleotide
  source 1..25: mol_type = other RNA
ttttccaggttcaagtgggatacta

SEQ ID NO: 94 - RNA, 25 nt, synthetic construct
  misc_feature 1..25: note = oligonucleotide
  source 1..25: mol_type = other RNA
tttccaggttcaagtgggatactag

SEQ ID NO: 95 - RNA, 25 nt, synthetic construct
  misc_feature 1..25: note = oligonucleotide
  source 1..25: mol_type = other RNA
ttccaggttcaagtgggatactagc

SEQ ID NO: 96 - RNA, 25 nt, synthetic construct
  misc_feature 1..25: note = oligonucleotide
  source 1..25: mol_type = other RNA
tccaggttcaagtgggatactagca

SEQ ID NO: 97 - RNA, 25 nt, synthetic construct
  misc_feature 1..25: note = oligonucleotide
  source 1..25: mol_type = other RNA
ccaggttcaagtgggatactagcaa

SEQ ID NO: 98 - RNA, 25 nt, synthetic construct
  misc_feature 1..25: note = oligonucleotide
  source 1..25: mol_type = other RNA
caggttcaagtgggatactagcaat

SEQ ID NO: 99 - RNA, 25 nt, synthetic construct
  misc_feature 1..25: note = oligonucleotide
  source 1..25: mol_type = other RNA
aggttcaagtgggatactagcaatg

SEQ ID NO: 100 - RNA, 25 nt, synthetic construct
  misc_feature 1..25: note = oligonucleotide
  source 1..25: mol_type = other RNA
ggttcaagtgggatactagcaatgt

SEQ ID NO: 101 - RNA, 25 nt, synthetic construct
  misc_feature 1..25: note = oligonucleotide
  source 1..25: mol_type = other RNA
gttcaagtgggatactagcaatgtt

SEQ ID NO: 102 - RNA, 25 nt, synthetic construct
  misc_feature 1..25: note = oligonucleotide
  source 1..25: mol_type = other RNA
ttcaagtgggatactagcaatgtta

SEQ ID NO: 103 - RNA, 25 nt, synthetic construct
  misc_feature 1..25: note = oligonucleotide
  source 1..25: mol_type = other RNA
tcaagtgggatactagcaatgttat

SEQ ID NO: 104 - RNA, 25 nt, synthetic construct
  misc_feature 1..25: note = oligonucleotide
  source 1..25: mol_type = other RNA
caagtgggatactagcaatgttatc

SEQ ID NO: 105 - RNA, 25 nt, synthetic construct
  misc_feature 1..25: note = oligonucleotide
  source 1..25: mol_type = other RNA
aagtgggatactagcaatgttatct

SEQ ID NO: 106 - RNA, 25 nt, synthetic construct
  misc_feature 1..25: note = oligonucleotide
  source 1..25: mol_type = other RNA
agtgggatactagcaatgttatctg

SEQ ID NO: 107 - RNA, 25 nt, synthetic construct
  misc_feature 1..25: note = oligonucleotide
  source 1..25: mol_type = other RNA
gtgggatactagcaatgttatctgc

SEQ ID NO: 108 - RNA, 25 nt, synthetic construct
  misc_feature 1..25: note = oligonucleotide
  source 1..25: mol_type = other RNA
tgggatactagcaatgttatctgct

SEQ ID NO: 109 - RNA, 25 nt, synthetic construct
  misc_feature 1..25: note = oligonucleotide
  source 1..25: mol_type = other RNA
gggatactagcaatgttatctgctt

SEQ ID NO: 110 - RNA, 25 nt, synthetic construct
  misc_feature 1..25: note = oligonucleotide
  source 1..25: mol_type = other RNA
ggatactagcaatgttatctgcttc

SEQ ID NO: 111 - RNA, 25 nt, synthetic construct
  misc_feature 1..25: note = oligonucleotide
  source 1..25: mol_type = other RNA
gatactagcaatgttatctgcttcc

SEQ ID NO: 112 - RNA, 25 nt, synthetic construct
  misc_feature 1..25: note = oligonucleotide
  source 1..25: mol_type = other RNA
atactagcaatgttatctgcttcct

SEQ ID NO: 113 - RNA, 25 nt, synthetic construct
  misc_feature 1..25: note = oligonucleotide
  source 1..25: mol_type = other RNA
tactagcaatgttatctgcttcctc

SEQ ID NO: 114 - RNA, 25 nt, synthetic construct
  misc_feature 1..25: note = oligonucleotide
  source 1..25: mol_type = other RNA
actagcaatgttatctgcttcctcc

SEQ ID NO: 115 - RNA, 25 nt, synthetic construct
  misc_feature 1..25: note = oligonucleotide
  source 1..25: mol_type = other RNA
ctagcaatgttatctgcttcctcca

SEQ ID NO: 116 - RNA, 25 nt, synthetic construct
  misc_feature 1..25: note = oligonucleotide
  source 1..25: mol_type = other RNA
tagcaatgttatctgcttcctccaa

SEQ ID NO: 117 - RNA, 25 nt, synthetic construct
  misc_feature 1..25: note = oligonucleotide
  source 1..25: mol_type = other RNA
agcaatgttatctgcttcctccaac

SEQ ID NO: 118 - RNA, 25 nt, synthetic construct
  misc_feature 1..25: note = oligonucleotide
  source 1..25: mol_type = other RNA
gcaatgttatctgcttcctccaacc

SEQ ID NO: 119 - RNA, 25 nt, synthetic construct
  misc_feature 1..25: note = oligonucleotide
  source 1..25: mol_type = other RNA
caatgttatctgcttcctccaacca

SEQ ID NO: 120 - RNA, 25 nt, synthetic construct
  misc_feature 1..25: note = oligonucleotide
  source 1..25: mol_type = other RNA
aatgttatctgcttcctccaaccat

SEQ ID NO: 121 - RNA, 25 nt, synthetic construct
  misc_feature 1..25: note = oligonucleotide
  source 1..25: mol_type = other RNA
atgttatctgcttcctccaaccata

SEQ ID NO: 122 - RNA, 25 nt, synthetic construct
  misc_feature 1..25: note = oligonucleotide
  source 1..25: mol_type = other RNA
tgttatctgcttcctccaaccataa

SEQ ID NO: 123 - RNA, 25 nt, synthetic construct
  misc_feature 1..25: note = oligonucleotide
  source 1..25: mol_type = other RNA
ccaatagtggtcagtccaggagcta

SEQ ID NO: 124 - RNA, 25 nt, synthetic construct
  misc_feature 1..25: note = oligonucleotide
  source 1..25: mol_type = other RNA
caatagtggtcagtccaggagctag

SEQ ID NO: 125 - RNA, 25 nt, synthetic construct
  misc_feature 1..25: note = oligonucleotide
  source 1..25: mol_type = other RNA
aatagtggtcagtccaggagctagg

SEQ ID NO: 126 - RNA, 25 nt, synthetic construct
  misc_feature 1..25: note = oligonucleotide
  source 1..25: mol_type = other RNA
atagtggtcagtccaggagctaggt

SEQ ID NO: 127 - RNA, 21 nt, synthetic construct
  misc_feature 1..21: note = oligonucleotide
  source 1..21: mol_type = other RNA
atagtggtcagtccaggagct

SEQ ID NO: 128 - RNA, 25 nt, synthetic construct
  misc_feature 1..25: note = oligonucleotide
  source 1..25: mol_type = other RNA
tagtggtcagtccaggagctaggtc

SEQ ID NO: 129 - RNA, 25 nt, synthetic construct
  misc_feature 1..25: note = oligonucleotide
  source 1..25: mol_type = other RNA
agtggtcagtccaggagctaggtca

SEQ ID NO: 130 - RNA, 25 nt, synthetic construct
  misc_feature 1..25: note = oligonucleotide
  source 1..25: mol_type = other RNA
gtggtcagtccaggagctaggtcag

SEQ ID NO: 131 - RNA, 25 nt, synthetic construct
  misc_feature 1..25: note = oligonucleotide
  source 1..25: mol_type = other RNA
tggtcagtccaggagctaggtcagg

SEQ ID NO: 132 - RNA, 25 nt, synthetic construct
  misc_feature 1..25: note = oligonucleotide
  source 1..25: mol_type = other RNA
ggtcagtccaggagctaggtcaggc

SEQ ID NO: 133 - RNA, 25 nt, synthetic construct
  misc_feature 1..25: note = oligonucleotide
  source 1..25: mol_type = other RNA
gtcagtccaggagctaggtcaggct

SEQ ID NO: 134 - RNA, 25 nt, synthetic construct
  misc_feature 1..25: note = oligonucleotide
  source 1..25: mol_type = other RNA
tcagtccaggagctaggtcaggctg

SEQ ID NO: 135 - RNA, 25 nt, synthetic construct
  misc_feature 1..25: note = oligonucleotide
  source 1..25: mol_type = other RNA
cagtccaggagctaggtcaggctgc

SEQ ID NO: 136 - RNA, 25 nt, synthetic construct
  misc_feature 1..25: note = oligonucleotide
  source 1..25: mol_type = other RNA
agtccaggagctaggtcaggctgct

SEQ ID NO: 137 - RNA, 25 nt, synthetic construct
  misc_feature 1..25: note = oligonucleotide
  source 1..25: mol_type = other RNA
gtccaggagctaggtcaggctgctt

SEQ ID NO: 138 - RNA, 25 nt, synthetic construct
  misc_feature 1..25: note = oligonucleotide
  source 1..25: mol_type = other RNA
tccaggagctaggtcaggctgcttt

SEQ ID NO: 139 - RNA, 25 nt, synthetic construct
  misc_feature 1..25: note = oligonucleotide
  source 1..25: mol_type = other RNA
ccaggagctaggtcaggctgctttg

SEQ ID NO: 140 - RNA, 25 nt, synthetic construct
  misc_feature 1..25: note = oligonucleotide
  source 1..25: mol_type = other RNA
caggagctaggtcaggctgctttgc

SEQ ID NO: 141 - RNA, 25 nt, synthetic construct
  misc_feature 1..25: note = oligonucleotide
  source 1..25: mol_type = other RNA
aggagctaggtcaggctgctttgcc

SEQ ID NO: 142 - RNA, 25 nt, synthetic construct
  misc_feature 1..25: note = oligonucleotide
  source 1..25: mol_type = other RNA
ggagctaggtcaggctgctttgccc

SEQ ID NO: 143 - RNA, 25 nt, synthetic construct
  misc_feature 1..25: note = oligonucleotide
  source 1..25: mol_type = other RNA
gagctaggtcaggctgctttgccct

SEQ ID NO: 144 - RNA, 25 nt, synthetic construct
  misc_feature 1..25: note = oligonucleotide
  source 1..25: mol_type = other RNA
agctaggtcaggctgctttgccctc

SEQ ID NO: 145 - RNA, 25 nt, synthetic construct
  misc_feature 1..25: note = oligonucleotide
  source 1..25: mol_type = other RNA
gctaggtcaggctgctttgccctca

SEQ ID NO: 146 - RNA, 25 nt, synthetic construct
  misc_feature 1..25: note = oligonucleotide
  source 1..25: mol_type = other RNA
ctaggtcaggctgctttgccctcag

SEQ ID NO: 147 - RNA, 25 nt, synthetic construct
  misc_feature 1..25: note = oligonucleotide
  source 1..25: mol_type = other RNA
taggtcaggctgctttgccctcagc

SEQ ID NO: 148 - RNA, 25 nt, synthetic construct
  misc_feature 1..25: note = oligonucleotide
  source 1..25: mol_type = other RNA
aggtcaggctgctttgccctcagct

SEQ ID NO: 149 - RNA, 25 nt, synthetic construct
  misc_feature 1..25: note = oligonucleotide
  source 1..25: mol_type = other RNA
ggtcaggctgctttgccctcagctc

SEQ ID NO: 150 - RNA, 25 nt, synthetic construct
  misc_feature 1..25: note = oligonucleotide
  source 1..25: mol_type = other RNA
gtcaggctgctttgccctcagctct

SEQ ID NO: 151 - RNA, 25 nt, synthetic construct
  misc_feature 1..25: note = oligonucleotide
  source 1..25: mol_type = other RNA
tcaggctgctttgccctcagctctt

SEQ ID NO: 152 - RNA, 25 nt, synthetic construct
  misc_feature 1..25: note = oligonucleotide
  source 1..25: mol_type = other RNA
caggctgctttgccctcagctcttg

SEQ ID NO: 153 - RNA, 25 nt, synthetic construct
  misc_feature 1..25: note = oligonucleotide
  source 1..25: mol_type = other RNA
aggctgctttgccctcagctcttga

SEQ ID NO: 154 - RNA, 25 nt, synthetic construct
  misc_feature 1..25: note = oligonucleotide
  source 1..25: mol_type = other RNA
ggctgctttgccctcagctcttgaa

SEQ ID NO: 155 - RNA, 25 nt, synthetic construct
  misc_feature 1..25: note = oligonucleotide
  source 1..25: mol_type = other RNA
gctgctttgccctcagctcttgaag

SEQ ID NO: 156 - RNA, 25 nt, synthetic construct
  misc_feature 1..25: note = oligonucleotide
  source 1..25: mol_type = other RNA
ctgctttgccctcagctcttgaagt

SEQ ID NO: 157 - RNA, 25 nt, synthetic construct
  misc_feature 1..25: note = oligonucleotide
  source 1..25: mol_type = other RNA
tgctttgccctcagctcttgaagta

SEQ ID NO: 158 - RNA, 25 nt, synthetic construct
  misc_feature 1..25: note = oligonucleotide
  source 1..25: mol_type = other RNA
gctttgccctcagctcttgaagtaa

SEQ ID NO: 159 - RNA, 25 nt, synthetic construct
  misc_feature 1..25: note = oligonucleotide
  source 1..25: mol_type = other RNA
ctttgccctcagctcttgaagtaaa

SEQ ID NO: 160 - RNA, 25 nt, synthetic construct
  misc_feature 1..25: note = oligonucleotide
  source 1..25: mol_type = other RNA
tttgccctcagctcttgaagtaaac

SEQ ID NO: 161 - RNA, 25 nt, synthetic construct
  misc_feature 1..25: note = oligonucleotide
  source 1..25: mol_type = other RNA
ttgccctcagctcttgaagtaaacg

SEQ ID NO: 162 - RNA, 25 nt, synthetic construct
  misc_feature 1..25: note = oligonucleotide
  source 1..25: mol_type = other RNA
tgccctcagctcttgaagtaaacgg

SEQ ID NO: 163 - RNA, 25 nt, synthetic construct
  misc_feature 1..25: note = oligonucleotide
  source 1..25: mol_type = other RNA
gccctcagctcttgaagtaaacggt

SEQ ID NO: 164 - RNA, 25 nt, synthetic construct
  misc_feature 1..25: note = oligonucleotide
  source 1..25: mol_type = other RNA
ccctcagctcttgaagtaaacggtt

SEQ ID NO: 165 - RNA, 20 nt, synthetic construct
  misc_feature 1..20: note = oligonucleotide
  source 1..20: mol_type = other RNA
cctcagctcttgaagtaaac

SEQ ID NO: 166 - RNA, 21 nt, synthetic construct
  misc_feature 1..21: note = oligonucleotide
  source 1..21: mol_type = other RNA
cctcagctcttgaagtaaacg

SEQ ID NO: 167 - RNA, 20 nt, synthetic construct
  misc_feature 1..20: note = oligonucleotide
  source 1..20: mol_type = other RNA
ctcagctcttgaagtaaacg

SEQ ID NO: 168 - RNA, 25 nt, synthetic construct
  misc_feature 1..25: note = oligonucleotide
  source 1..25: mol_type = other RNA
gtacctccaacatcaaggaagatgg

SEQ ID NO: 169 - RNA, 25 nt, synthetic construct
  misc_feature 1..25: note = oligonucleotide
  source 1..25: mol_type = other RNA
tacctccaacatcaaggaagatggc

SEQ ID NO: 170 - RNA, 25 nt, synthetic construct
  misc_feature 1..25: note = oligonucleotide
  source 1..25: mol_type = other RNA
acctccaacatcaaggaagatggca

SEQ ID NO: 171 - RNA, 25 nt, synthetic construct
  misc_feature 1..25: note = oligonucleotide
  source 1..25: mol_type = other RNA
cctccaacatcaaggaagatggcat

SEQ ID NO: 172 - RNA, 25 nt, synthetic construct
  misc_feature 1..25: note = oligonucleotide
  source 1..25: mol_type = other RNA
ctccaacatcaaggaagatggcatt

SEQ ID NO: 173 - RNA, 25 nt, synthetic construct
  misc_feature 1..25: note = oligonucleotide
  source 1..25: mol_type = other RNA
tccaacatcaaggaagatggcattt

SEQ ID NO: 174 - RNA, 25 nt, synthetic construct
  misc_feature 1..25: note = oligonuclelotide
  source 1..25: mol_type = other RNA
ccaacatcaaggaagatggcatttc

SEQ ID NO: 175 - RNA, 25 nt, synthetic construct
  misc_feature 1..25: note = oligonucleotide
  source 1..25: mol_type = other RNA
caacatcaaggaagatggcatttct

SEQ ID NO: 176 - RNA, 25 nt, synthetic construct
  misc_feature 1..25: note = oligonucleotide
  source 1..25: mol_type = other RNA
aacatcaaggaagatggcatttcta

SEQ ID NO: 177 - RNA, 25 nt, synthetic construct
  misc_feature 1..25: note = oligonucleotide
  source 1..25: mol_type = other RNA
acatcaaggaagatggcatttctag

SEQ ID NO: 178 - RNA, 25 nt, synthetic construct
  misc_feature 1..25: note = oligonucleotide
  source 1..25: mol_type = other RNA
catcaaggaagatggcatttctagt

SEQ ID NO: 179 - RNA, 25 nt, synthetic construct
  misc_feature 1..25: note = oligonucleotide
  source 1..25: mol_type = other RNA
atcaaggaagatggcatttctagtt

SEQ ID NO: 180 - RNA, 25 nt, synthetic construct
  misc_feature 1..25: note = oligonucleotide
  source 1..25: mol_type = other RNA
tcaaggaagatggcatttctagttt

SEQ ID NO: 181 - RNA, 25 nt, synthetic construct
  misc_feature 1..25: note = oligonucleotide
  source 1..25: mol_type = other RNA
caaggaagatggcatttctagtttg

SEQ ID NO: 182 - RNA, 25 nt, synthetic construct
  misc_feature 1..25: note = oligonucleotide
  source 1..25: mol_type = other RNA
aaggaagatggcatttctagtttgg

SEQ ID NO: 183 - RNA, 25 nt, synthetic construct
  misc_feature 1..25: note = oligonucleotide
  source 1..25: mol_type = other RNA
aggaagatggcatttctagtttgga

SEQ ID NO: 184 - RNA, 25 nt, synthetic construct
  misc_feature 1..25: note = oligonucleotide
  source 1..25: mol_type = other RNA
ggaagatggcatttctagtttggag

SEQ ID NO: 185 - RNA, 25 nt, synthetic construct
  misc_feature 1..25: note = oligonucleotide
  source 1..25: mol_type = other RNA
gaagatggcatttctagtttggaga

SEQ ID NO: 186 - RNA, 25 nt, synthetic construct
  misc_feature 1..25: note = oligonucleotide
  source 1..25: mol_type = other RNA
aagatggcatttctagtttggagat

SEQ ID NO: 187 - RNA, 25 nt, synthetic construct
  misc_feature 1..25: note = oligonucleotide
  source 1..25: mol_type = other RNA
agatggcatttctagtttggagatg

SEQ ID NO: 188 - RNA, 25 nt, synthetic construct
  misc_feature 1..25: note = oligonucleotide
  source 1..25: mol_type = other RNA
gatggcatttctagtttggagatgg

SEQ ID NO: 189 - RNA, 25 nt, synthetic construct
  misc_feature 1..25: note = oligonucleotide
  source 1..25: mol_type = other RNA
atggcatttctagtttggagatggc

SEQ ID NO: 190 - RNA, 25 nt, synthetic construct
  misc_feature 1..25: note = oligonucleotide
  source 1..25: mol_type = other RNA
tggcatttctagtttggagatggca

SEQ ID NO: 191 - RNA, 25 nt, synthetic construct
  misc_feature 1..25: note = oligonucleotide
  source 1..25: mol_type = other RNA
ggcatttctagtttggagatggcag

SEQ ID NO: 192 - RNA, 25 nt, synthetic construct
  misc_feature 1..25: note = oligonucleotide
  source 1..25: mol_type = other RNA
gcatttctagtttggagatggcagt

SEQ ID NO: 193 - RNA, 25 nt, synthetic construct
  misc_feature 1..25: note = oligonucleotide
  source 1..25: mol_type = other RNA
catttctagtttggagatggcagtt

SEQ ID NO: 194 - RNA, 25 nt, synthetic construct
  misc_feature 1..25: note = oligonucleotide
  source 1..25: mol_type = other RNA
atttctagtttggagatggcagttt

SEQ ID NO: 195 - RNA, 25 nt, synthetic construct
  misc_feature 1..25: note = oligonucleotide
  source 1..25: mol_type = other RNA
tttctagtttggagatggcagtttc

SEQ ID NO: 196 - RNA, 25 nt, synthetic construct
  misc_feature 1..25: note = oligonucleotide
  source 1..25: mol_type = other RNA
ttctagtttggagatggcagtttcc

SEQ ID NO: 197 - RNA, 25 nt, synthetic construct
  misc_feature 1..25: note = oligonucleotide
  source 1..25: mol_type = other RNA
tctagtttggagatggcagtttcct

SEQ ID NO: 198 - RNA, 25 nt, synthetic construct
  misc_feature 1..25: note = oligonucleotide
  source 1..25: mol_type = other RNA
ctagtttggagatggcagtttcctt

SEQ ID NO: 199 - RNA, 25 nt, synthetic construct
  misc_feature 1..25: note = oligonucleotide
  source 1..25: mol_type = other RNA
tagtttggagatggcagtttcctta

SEQ ID NO: 200 - RNA, 25 nt, synthetic construct
  misc_feature 1..25: note = oligonucleotide
  source 1..25: mol_type = other RNA
agtttggagatggcagtttccttag

SEQ ID NO: 201 - RNA, 25 nt, synthetic construct
  misc_feature 1..25: note = oligonucleotide
  source 1..25: mol_type = other RNA
gtttggagatggcagtttccttagt

SEQ ID NO: 202 - RNA, 25 nt, synthetic construct
  misc_feature 1..25: note = oligonucleotide
  source 1..25: mol_type = other RNA
tttggagatggcagtttccttagta

SEQ ID NO: 203 - RNA, 25 nt, synthetic construct
  misc_feature 1..25: note = oligonucleotide
  source 1..25: mol_type = other RNA
ttggagatggcagtttccttagtaa

SEQ ID NO: 204 - RNA, 25 nt, synthetic construct
  misc_feature 1..25: note = oligonucleotide
  source 1..25: mol_type = other RNA
tggagatggcagtttccttagtaac

SEQ ID NO: 205 - RNA, 25 nt, synthetic construct
  misc_feature 1..25: note = oligonucleotide
  source 1..25: mol_type = other RNA
gagatggcagtttccttagtaacca

SEQ ID NO: 206 - RNA, 25 nt, synthetic construct
  misc_feature 1..25: note = oligonucleotide
  source 1..25: mol_type = other RNA
agatggcagtttccttagtaaccac

SEQ ID NO: 207 - RNA, 25 nt, synthetic construct
  misc_feature 1..25: note = oligonucleotide
  source 1..25: mol_type = other RNA
gatggcagtttccttagtaaccaca

SEQ ID NO: 208 - RNA, 25 nt, synthetic construct
  misc_feature 1..25: note = oligonucleotide
  source 1..25: mol_type = other RNA
atggcagtttccttagtaaccacag

SEQ ID NO: 209 - RNA, 25 nt, synthetic construct
  misc_feature 1..25: note = oligonucleotide
  source 1..25: mol_type = other RNA
tggcagtttccttagtaaccacagg

SEQ ID NO: 210 - RNA, 25 nt, synthetic construct
  misc_feature 1..25: note = oligonucleotide
  source 1..25: mol_type = other RNA
ggcagtttccttagtaaccacaggt

SEQ ID NO: 211 - RNA, 25 nt, synthetic construct
  misc_feature 1..25: note = oligonucleotide
  source 1..25: mol_type = other RNA
gcagtttccttagtaaccacaggtt

SEQ ID NO: 212 - RNA, 25 nt, synthetic construct
  misc_feature 1..25: note = oligonucleotide
  source 1..25: mol_type = other RNA
cagtttccttagtaaccacaggttg

SEQ ID NO: 213 - RNA, 25 nt, synthetic construct
  misc_feature 1..25: note = oligonucleotide
  source 1..25: mol_type = other RNA
agtttccttagtaaccacaggttgt

SEQ ID NO: 214 - RNA, 25 nt, synthetic construct
  misc_feature 1..25: note = oligonucleotide
  source 1..25: mol_type = other RNA
gtttccttagtaaccacaggttgtg

SEQ ID NO: 215 - RNA, 25 nt, synthetic construct
  misc_feature 1..25: note = oligonucleotide
  source 1..25: mol_type = other RNA
tttccttagtaaccacaggttgtgt

SEQ ID NO: 216 - RNA, 25 nt, synthetic construct
  misc_feature 1..25: note = oligonucleotide
  source 1..25: mol_type = other RNA
ttccttagtaaccacaggttgtgtc

SEQ ID NO: 217 - RNA, 25 nt, synthetic construct
  misc_feature 1..25: note = oligonucleotide
  source 1..25: mol_type = other RNA
tccttagtaaccacaggttgtgtca

SEQ ID NO: 218 - RNA, 25 nt, synthetic construct
  misc_feature 1..25: note = oligonucleotide
  source 1..25: mol_type = other RNA
ccttagtaaccacaggttgtgtcac

SEQ ID NO: 219 - RNA, 25 nt, synthetic construct
  misc_feature 1..25: note = oligonucleotide
  source 1..25: mol_type = other RNA
cttagtaaccacaggttgtgtcacc

SEQ ID NO: 220 - RNA, 25 nt, synthetic construct
  misc_feature 1..25: note = oligonucleotide
  source 1..25: mol_type = other RNA
ttagtaaccacaggttgtgtcacca

SEQ ID NO: 221 - RNA, 25 nt, synthetic construct
  misc_feature 1..25: note = oligonucleotide
  source 1..25: mol_type = other RNA
tagtaaccacaggttgtgtcaccag

SEQ ID NO: 222 - RNA, 25 nt, synthetic construct
  misc_feature 1..25: note = oligonucleotide
  source 1..25: mol_type = other RNA
agtaaccacaggttgtgtcaccaga

SEQ ID NO: 223 - RNA, 25 nt, synthetic construct
  misc_feature 1..25: note = oligonucleotide
  source 1..25: mol_type = other RNA
gtaaccacaggttgtgtcaccagag

SEQ ID NO: 224 - RNA, 25 nt, synthetic construct
  misc_feature 1..25: note = oligonucleotide
  source 1..25: mol_type = other RNA
taaccacaggttgtgtcaccagagt

SEQ ID NO: 225 - RNA, 25 nt, synthetic construct
  misc_feature 1..25: note = oligonucleotide
  source 1..25: mol_type = other RNA
aaccacaggttgtgtcaccagagta

SEQ ID NO: 226 - RNA, 25 nt, synthetic construct
  misc_feature 1..25: note = oligonucleotide
  source 1..25: mol_type = other RNA
accacaggttgtgtcaccagagtaa

SEQ ID NO: 227 - RNA, 25 nt, synthetic construct
  misc_feature 1..25: note = oligonucleotide
  source 1..25: mol_type = other RNA
ccacaggttgtgtcaccagagtaac

SEQ ID NO: 228 - RNA, 25 nt, synthetic construct
  misc_feature 1..25: note = oligonucleotide
  source 1..25: mol_type = other RNA
cacaggttgtgtcaccagagtaaca

SEQ ID NO: 229 - RNA, 25 nt, synthetic construct
  misc_feature 1..25: note = oligonucleotide
  source 1..25: mol_type = other RNA
acaggttgtgtcaccagagtaacag

SEQ ID NO: 230 - RNA, 25 nt, synthetic construct
  misc_feature 1..25: note = oligonucleotide
  source 1..25: mol_type = other RNA
caggttgtgtcaccagagtaacagt

SEQ ID NO: 231 - RNA, 25 nt, synthetic construct
  misc_feature 1..25: note = oligonucleotide
  source 1..25: mol_type = other RNA
aggttgtgtcaccagagtaacagtc

SEQ ID NO: 232 - RNA, 25 nt, synthetic construct
  misc_feature 1..25: note = oligonucleotide
  source 1..25: mol_type = other RNA
ggttgtgtcaccagagtaacagtct

SEQ ID NO: 233 - RNA, 25 nt, synthetic construct
  misc_feature 1..25: note = oligonucleotide
  source 1..25: mol_type = other RNA
gttgtgtcaccagagtaacagtctg

SEQ ID NO: 234 - RNA, 25 nt, synthetic construct
  misc_feature 1..25: note = oligonucleotide
  source 1..25: mol_type = other RNA
ttgtgtcaccagagtaacagtctga

SEQ ID NO: 235 - RNA, 25 nt, synthetic construct
  misc_feature 1..25: note = oligonucleotide
  source 1..25: mol_type = other RNA
tgtgtcaccagagtaacagtctgag

SEQ ID NO: 236 - RNA, 25 nt, synthetic construct
  misc_feature 1..25: note = oligonucleotide
  source 1..25: mol_type = other RNA
gtgtcaccagagtaacagtctgagt

SEQ ID NO: 237 - RNA, 25 nt, synthetic construct
  misc_feature 1..25: note = oligonucleotide
  source 1..25: mol_type = other RNA
tgtcaccagagtaacagtctgagta

SEQ ID NO: 238 - RNA, 25 nt, synthetic construct
  misc_feature 1..25: note = oligonucleotide
  source 1..25: mol_type = other RNA
gtcaccagagtaacagtctgagtag

SEQ ID NO: 239 - RNA, 25 nt, synthetic construct
  misc_feature 1..25: note = oligonucleotide
  source 1..25: mol_type = other RNA
tcaccagagtaacagtctgagtagg

SEQ ID NO: 240 - RNA, 25 nt, synthetic construct
  misc_feature 1..25: note = oligonucleotide
  source 1..25: mol_type = other RNA
caccagagtaacagtctgagtagga

SEQ ID NO: 241 - RNA, 25 nt, synthetic construct
  misc_feature 1..25: note = oligonucleotide
  source 1..25: mol_type = other RNA
accagagtaacagtctgagtaggag

SEQ ID NO: 242 - RNA, 25 nt, synthetic construct
  misc_feature 1..25: note = oligonucleotide
  source 1..25: mol_type = other RNA
agcctcttgattgctggtcttgttt

SEQ ID NO: 243 - RNA, 25 nt, synthetic construct
  misc_feature 1..25: note = oligonucleotide
  source 1..25: mol_type = other RNA
gcctcttgattgctggtcttgtttt

SEQ ID NO: 244 - RNA, 25 nt, synthetic construct
  misc_feature 1..25: note = oligonucleotide
  source 1..25: mol_type = other RNA
cctcttgattgctggtcttgttttt

SEQ ID NO: 245 - RNA, 20 nt, synthetic construct
  misc_feature 1..20: note = oligonucleotide
  source 1..20: mol_type = other RNA
cctcttgattgctggtcttg

SEQ ID NO: 246 - RNA, 25 nt, synthetic construct
  misc_feature 1..25: note = oligonucleotide
  source 1..25: mol_type = other RNA
ctcttgattgctggtcttgtttttc

SEQ ID NO: 247 - RNA, 25 nt, synthetic construct
  misc_feature 1..25: note = oligonucleotide
  source 1..25: mol_type = other RNA
tcttgattgctggtcttgtttttca

SEQ ID NO: 248 - RNA, 25 nt, synthetic construct
  misc_feature 1..25: note = oligonucleotide
  source 1..25: mol_type = other RNA
cttgattgctggtcttgtttttcaa

SEQ ID NO: 249 - RNA, 25 nt, synthetic construct
  misc_feature 1..25: note = oligonucleotide
  source 1..25: mol_type = other RNA
ttgattgctggtcttgtttttcaaa

SEQ ID NO: 250 - RNA, 25 nt, synthetic construct
  misc_feature 1..25: note = oligonucleotide
  source 1..25: mol_type = other RNA
tgattgctggtcttgtttttcaaat

SEQ ID NO: 251 - RNA, 25 nt, synthetic construct
  misc_feature 1..25: note = oligonucleotide
  source 1..25: mol_type = other RNA
gattgctggtcttgtttttcaaatt

SEQ ID NO: 252 - RNA, 20 nt, synthetic construct
  misc_feature 1..20: note = oligonucleotide
  source 1..20: mol_type = other RNA
gattgctggtcttgtttttc

SEQ ID NO: 253 - RNA, 25 nt, synthetic construct
  misc_feature 1..25: note = oligonucleotide
  source 1..25: mol_type = other RNA
attgctggtcttgtttttcaaattt

SEQ ID NO: 254 - RNA, 25 nt, synthetic construct
  misc_feature 1..25: note = oligonucleotide
  source 1..25: mol_type = other RNA
ttgctggtcttgtttttcaaatttt

SEQ ID NO: 255 - RNA, 25 nt, synthetic construct
  misc_feature 1..25: note = oligonucleotide
  source 1..25: mol_type = other RNA
tgctggtcttgtttttcaaattttg

SEQ ID NO: 256 - RNA, 25 nt, synthetic construct
  misc_feature 1..25: note = oligonucleotide
  source 1..25: mol_type = other RNA
gctggtcttgtttttcaaattttgg

SEQ ID NO: 257 - RNA, 25 nt, synthetic construct
  misc_feature 1..25: note = oligonucleotide
  source 1..25: mol_type = other RNA
ctggtcttgtttttcaaattttggg

SEQ ID NO: 258 - RNA, 25 nt, synthetic construct
  misc_feature 1..25: note = oligonucleotide
  source 1..25: mol_type = other RNA
tggtcttgtttttcaaattttgggc

SEQ ID NO: 259 - RNA, 25 nt, synthetic construct
  misc_feature 1..25: note = oligonucleotide
  source 1..25: mol_type = other RNA
ggtcttgtttttcaaattttgggca

SEQ ID NO: 260 - RNA, 25 nt, synthetic construct
  misc_feature 1..25: note = oligonucleotide
  source 1..25: mol_type = other RNA
gtcttgtttttcaaattttgggcag

SEQ ID NO: 261 - RNA, 25 nt, synthetic construct
  misc_feature 1..25: note = oligonucleotide
  source 1..25: mol_type = other RNA
tcttgtttttcaaattttgggcagc

SEQ ID NO: 262 - RNA, 25 nt, synthetic construct
  misc_feature 1..25: note = oligonucleotide
  source 1..25: mol_type = other RNA
cttgtttttcaaattttgggcagcg

SEQ ID NO: 263 - RNA, 25 nt, synthetic construct
  misc_feature 1..25: note = oligonucleotide
  source 1..25: mol_type = other RNA
ttgtttttcaaattttgggcagcgg

SEQ ID NO: 264 - RNA, 25 nt, synthetic construct
  misc_feature 1..25: note = oligonucleotide
  source 1..25: mol_type = other RNA
tgtttttcaaattttgggcagcggt

SEQ ID NO: 265 - RNA, 25 nt, synthetic construct
  misc_feature 1..25: note = oligonucleotide
  source 1..25: mol_type = other RNA
gtttttcaaattttgggcagcggta

SEQ ID NO: 266 - RNA, 25 nt, synthetic construct
  misc_feature 1..25: note = oligonucleotide
  source 1..25: mol_type = other RNA
tttttcaaattttgggcagcggtaa

SEQ ID NO: 267 - RNA, 25 nt, synthetic construct
  misc_feature 1..25: note = oligonucleotide
  source 1..25: mol_type = other RNA
ttttcaaattttgggcagcggtaat

SEQ ID NO: 268 - RNA, 25 nt, synthetic construct
  misc_feature 1..25: note = oligonucleotide
  source 1..25: mol_type = other RNA
tttcaaattttgggcagcggtaatg

SEQ ID NO: 269 - RNA, 25 nt, synthetic construct
  misc_feature 1..25: note = oligonucleotide
  source 1..25: mol_type = other RNA
ttcaaattttgggcagcggtaatga

SEQ ID NO: 270 - RNA, 25 nt, synthetic construct
  misc_feature 1..25: note = oligonucleotide
  source 1..25: mol_type = other RNA
tcaaattttgggcagcggtaatgag

SEQ ID NO: 271 - RNA, 25 nt, synthetic construct
  misc_feature 1..25: note = oligonucleotide
  source 1..25: mol_type = other RNA
caaattttgggcagcggtaatgagt

SEQ ID NO: 272 - RNA, 25 nt, synthetic construct
  misc_feature 1..25: note = oligonucleotide
  source 1..25: mol_type = other RNA
aaattttgggcagcggtaatgagtt

SEQ ID NO: 273 - RNA, 25 nt, synthetic construct
  misc_feature 1..25: note = oligonucleotide
  source 1..25: mol_type = other RNA
aattttgggcagcggtaatgagttc

SEQ ID NO: 274 - RNA, 25 nt, synthetic construct
  misc_feature 1..25: note = oligonucleotide
  source 1..25: mol_type = other RNA
attttgggcagcggtaatgagttct

SEQ ID NO: 275 - RNA, 25 nt, synthetic construct
  misc_feature 1..25: note = oligonucleotide
  source 1..25: mol_type = other RNA
ttttgggcagcggtaatgagttctt

SEQ ID NO: 276 - RNA, 25 nt, synthetic construct
  misc_feature 1..25: note = oligonucleotide
  source 1..25: mol_type = other RNA
tttgggcagcggtaatgagttcttc

SEQ ID NO: 277 - RNA, 25 nt, synthetic construct
  misc_feature 1..25: note = oligonucleotide
  source 1..25: mol_type = other RNA
ttgggcagcggtaatgagttcttcc

SEQ ID NO: 278 - RNA, 25 nt, synthetic construct
  misc_feature 1..25: note = oligonucleotide
  source 1..25: mol_type = other RNA
tgggcagcggtaatgagttcttcca

SEQ ID NO: 279 - RNA, 25 nt, synthetic construct
  misc_feature 1..25: note = oligonucleotide
  source 1..25: mol_type = other RNA
gggcagcggtaatgagttcttccaa

SEQ ID NO: 280 - RNA, 25 nt, synthetic construct
  misc_feature 1..25: note = oligonucleotide
  source 1..25: mol_type = other RNA
ggcagcggtaatgagttcttccaac

SEQ ID NO: 281 - RNA, 25 nt, synthetic construct
  misc_feature 1..25: note = oligonucleotide
  source 1..25: mol_type = other RNA
gcagcggtaatgagttcttccaact

SEQ ID NO: 282 - RNA, 25 nt, synthetic construct
  misc_feature 1..25: note = oligonucleotide
  source 1..25: mol_type = other RNA
cagcggtaatgagttcttccaactg

SEQ ID NO: 283 - RNA, 25 nt, synthetic construct
  misc_feature 1..25: note = oligonucleotide
  source 1..25: mol_type = other RNA
agcggtaatgagttcttccaactgg

SEQ ID NO: 284 - RNA, 25 nt, synthetic construct
  misc_feature 1..25: note = oligonucleotide
  source 1..25: mol_type = other RNA
gcggtaatgagttcttccaactggg

SEQ ID NO: 285 - RNA, 25 nt, synthetic construct
  misc_feature 1..25: note = oligonucleotide
  source 1..25: mol_type = other RNA
cggtaatgagttcttccaactgggg

SEQ ID NO: 286 - RNA, 25 nt, synthetic construct
  misc_feature 1..25: note = oligonucleotide
  source 1..25: mol_type = other RNA
ggtaatgagttcttccaactgggga

SEQ ID NO: 287 - RNA, 22 nt, synthetic construct
  misc_feature 1..22: note = oligonucleotide
  source 1..22: mol_type = other RNA
ggtaatgagttcttccaactgg

SEQ ID NO: 288 - RNA, 25 nt, synthetic construct
  misc_feature 1..25: note = oligonucleotide
  source 1..25: mol_type = other RNA
gtaatgagttcttccaactggggac

SEQ ID NO: 289 - RNA, 25 nt, synthetic construct
  misc_feature 1..25: note = oligonucleotide
  source 1..25: mol_type = other RNA
taatgagttcttccaactggggacg

SEQ ID NO: 290 - RNA, 25 nt, synthetic construct
  misc_feature 1..25: note = oligonucleotide
  source 1..25: mol_type = other RNA
aatgagttcttccaactggggacgc

SEQ ID NO: 291 - RNA, 25 nt, synthetic construct
  misc_feature 1..25: note = oligonucleotide
  source 1..25: mol_type = other RNA
atgagttcttccaactggggacgcc

SEQ ID NO: 292 - RNA, 25 nt, synthetic construct
  misc_feature 1..25: note = oligonucleotide
  source 1..25: mol_type = other RNA
tgagttcttccaactggggacgcct

SEQ ID NO: 293 - RNA, 25 nt, synthetic construct
  misc_feature 1..25: note = oligonucleotide
  source 1..25: mol_type = other RNA
gagttcttccaactggggacgcctc

SEQ ID NO: 294 - RNA, 25 nt, synthetic construct
  misc_feature 1..25: note = oligonucleotide
  source 1..25: mol_type = other RNA
agttcttccaactggggacgcctct

SEQ ID NO: 295 - RNA, 25 nt, synthetic construct
  misc_feature 1..25: note = oligonucleotide
  source 1..25: mol_type = other RNA
gttcttccaactggggacgcctctg

SEQ ID NO: 296 - RNA, 25 nt, synthetic construct
  misc_feature 1..25: note = oligonucleotide
  source 1..25: mol_type = other RNA
ttcttccaactggggacgcctctgt

SEQ ID NO: 297 - RNA, 25 nt, synthetic construct
  misc_feature 1..25: note = oligonucleotide
  source 1..25: mol_type = other RNA
tcttccaactggggacgcctctgtt

SEQ ID NO: 298 - RNA, 25 nt, synthetic construct
  misc_feature 1..25: note = oligonucleotide
  source 1..25: mol_type = other RNA
cttccaactggggacgcctctgttc

SEQ ID NO: 299 - RNA, 25 nt, synthetic construct
  misc_feature 1..25: note = oligonucleotide
  source 1..25: mol_type = other RNA
ttccaactggggacgcctctgttcc

SEQ ID NO: 300 - RNA, 25 nt, synthetic construct
  misc_feature 1..25: note = oligonucleotide
  source 1..25: mol_type = other RNA
tccaactggggacgcctctgttcca

SEQ ID NO: 301 - RNA, 25 nt, synthetic construct
  misc_feature 1..25: note = oligonucleotide
  source 1..25: mol_type = other RNA
ccaactggggacgcctctgttccaa

SEQ ID NO: 302 - RNA, 25 nt, synthetic construct
  misc_feature 1..25: note = oligonucleotide
  source 1..25: mol_type = other RNA
caactggggacgcctctgttccaaa

SEQ ID NO: 303 - RNA, 25 nt, synthetic construct
  misc_feature 1..25: note = oligonucleotide
  source 1..25: mol_type = other RNA
aactggggacgcctctgttccaaat

SEQ ID NO: 304 - RNA, 25 nt, synthetic construct
  misc_feature 1..25: note = oligonucleotide
  source 1..25: mol_type = other RNA
actggggacgcctctgttccaaatc

SEQ ID NO: 305 - RNA, 25 nt, synthetic construct
  misc_feature 1..25: note = oligonucleotide
  source 1..25: mol_type = other RNA
ctggggacgcctctgttccaaatcc

SEQ ID NO: 306 - RNA, 25 nt, synthetic construct
  misc_feature 1..25: note = oligonucleotide
  source 1..25: mol_type = other RNA
tggggacgcctctgttccaaatcct

SEQ ID NO: 307 - RNA, 25 nt, synthetic construct
  misc_feature 1..25: note = oligonucleotide
  source 1..25: mol_type = other RNA
ggggacgcctctgttccaaatcctg

SEQ ID NO: 308 - RNA, 25 nt, synthetic construct
  misc_feature 1..25: note = oligonucleotide
  source 1..25: mol_type = other RNA
gggacgcctctgttccaaatcctgc

SEQ ID NO: 309 - RNA, 25 nt, synthetic construct
  misc_feature 1..25: note = oligonucleotide
  source 1..25: mol_type = other RNA
ggacgcctctgttccaaatcctgca

SEQ ID NO: 310 - RNA, 25 nt, synthetic construct
  misc_feature 1..25: note = oligonucleotide
  source 1..25: mol_type = other RNA
gacgcctctgttccaaatcctgcat

SEQ ID NO: 311 - RNA, 25 nt, synthetic construct
  misc_feature 1..25: note = oligonucleotide
  source 1..25: mol_type = other RNA
ctctggcctgtcctaagacctgctc

SEQ ID NO: 312 - RNA, 25 nt, synthetic construct
  misc_feature 1..25: note = oligonucleotide
  source 1..25: mol_type = other RNA
tctggcctgtcctaagacctgctca

SEQ ID NO: 313 - skipped

SEQ ID NO: 314 - RNA, 25 nt, synthetic construct
  misc_feature 1..25: note = oligonucleotide
  source 1..25: mol_type = other RNA
tggcctgtcctaagacctgctcagc

SEQ ID NO: 315 - RNA, 25 nt, synthetic construct
  misc_feature 1..25: note = oligonucleotide
  source 1..25: mol_type = other RNA
ggcctgtcctaagacctgctcagct

SEQ ID NO: 316 - RNA, 25 nt, synthetic construct
  misc_feature 1..25: note = oligonucleotide
  source 1..25: mol_type = other RNA
gcctgtcctaagacctgctcagctt

SEQ ID NO: 317 - RNA, 25 nt, synthetic construct
  misc_feature 1..25: note = oligonucleotide
  source 1..25: mol_type = other RNA
cctgtcctaagacctgctcagcttc

SEQ ID NO: 318 - RNA, 25 nt, synthetic construct
  misc_feature 1..25: note = oligonucleotide
  source 1..25: mol_type = other RNA
ctgtcctaagacctgctcagcttct

SEQ ID NO: 319 - RNA, 25 nt, synthetic construct
  misc_feature 1..25: note = oligonucleotide
  source 1..25: mol_type = other RNA
tgtcctaagacctgctcagcttctt

SEQ ID NO: 320 - RNA, 25 nt, synthetic construct
  misc_feature 1..25: note = oligonucleotide
  source 1..25: mol_type = other RNA
gtcctaagacctgctcagcttcttc

SEQ ID NO: 321 - RNA, 25 nt, synthetic construct
  misc_feature 1..25: note = oligonucleotide
  source 1..25: mol_type = other RNA
tcctaagacctgctcagcttcttcc

SEQ ID NO: 322 - RNA, 25 nt, synthetic construct
  misc_feature 1..25: note = oligonucleotide
  source 1..25: mol_type = other RNA
cctaagacctgctcagcttcttcct

SEQ ID NO: 323 - RNA, 25 nt, synthetic construct
  misc_feature 1..25: note = oligonucleotide
  source 1..25: mol_type = other RNA
ctaagacctgctcagcttcttcctt

SEQ ID NO: 324 - RNA, 25 nt, synthetic construct
  misc_feature 1..25: note = oligonucleotide
  source 1..25: mol_type = other RNA
taagacctgctcagcttcttcctta

SEQ ID NO: 325 - RNA, 25 nt, synthetic construct
  misc_feature 1..25: note = oligonucleotide
  source 1..25: mol_type = other RNA
aagacctgctcagcttcttccttag

SEQ ID NO: 326 - RNA, 25 nt, synthetic construct
  misc_feature 1..25: note = oligonucleotide
  source 1..25: mol_type = other RNA
agacctgctcagcttcttccttagc

SEQ ID NO: 327 - RNA, 25 nt, synthetic construct
  misc_feature 1..25: note = oligonucleotide
  source 1..25: mol_type = other RNA
gacctgctcagcttcttccttagct

SEQ ID NO: 328 - RNA, 25 nt, synthetic construct
  misc_feature 1..25: note = oligonucleotide
  source 1..25: mol_type = other RNA
acctgctcagcttcttccttagctt

SEQ ID NO: 329 - RNA, 25 nt, synthetic construct
  misc_feature 1..25: note = oligonucleotide
  source 1..25: mol_type = other RNA
cctgctcagcttcttccttagcttc

SEQ ID NO: 330 - RNA, 25 nt, synthetic construct
  misc_feature 1..25: note = oligonucleotide
  source 1..25: mol_type = other RNA
ctgctcagcttcttccttagcttcc

SEQ ID NO: 331 - RNA, 25 nt, synthetic construct
  misc_feature 1..25: note = oligonucleotide
  source 1..25: mol_type = other RNA
tgctcagcttcttccttagcttcca

SEQ ID NO: 332 - RNA, 25 nt, synthetic construct
  misc_feature 1..25: note = oligonucleotide
  source 1..25: mol_type = other RNA
gctcagcttcttccttagcttccag

SEQ ID NO: 333 - RNA, 25 nt, synthetic construct
  misc_feature 1..25: note = oligonucleotide
  source 1..25: mol_type = other RNA
ctcagcttcttccttagcttccagc

SEQ ID NO: 334 - RNA, 25 nt, synthetic construct
  misc_feature 1..25: note = oligonucleotide
  source 1..25: mol_type = other RNA
tcagcttcttccttagcttccagcc

SEQ ID NO: 335 - RNA, 25 nt, synthetic construct
  misc_feature 1..25: note = oligonucleotide
  source 1..25: mol_type = other RNA
cagcttcttccttagcttccagcca

SEQ ID NO: 336 - RNA, 25 nt, synthetic construct
  misc_feature 1..25: note = oligonucleotide
  source 1..25: mol_type = other RNA
agcttcttccttagcttccagccat

SEQ ID NO: 337 - RNA, 25 nt, synthetic construct
  misc_feature 1..25: note = oligonucleotide
  source 1..25: mol_type = other RNA
gcttcttccttagcttccagccatt

SEQ ID NO: 338 - RNA, 25 nt, synthetic construct
  misc_feature 1..25: note = oligonucleotide
  source 1..25: mol_type = other RNA
cttcttccttagcttccagccattg

SEQ ID NO: 339 - RNA, 25 nt, synthetic construct
  misc_feature 1..25: note = oligonucleotide
  source 1..25: mol_type = other RNA
ttcttccttagcttccagccattgt

SEQ ID NO: 340 - RNA, 25 nt, synthetic construct
  misc_feature 1..25: note = oligonucleotide
  source 1..25: mol_type = other RNA
tcttccttagcttccagccattgtg

SEQ ID NO: 341 - RNA, 25 nt, synthetic construct
  misc_feature 1..25: note = oligonucleotide
  source 1..25: mol_type = other RNA
cttccttagcttccagccattgtgt

SEQ ID NO: 342 - RNA, 25 nt, synthetic construct
  misc_feature 1..25: note = oligonucleotide
  source 1..25: mol_type = other RNA
ttccttagcttccagccattgtgtt

SEQ ID NO: 343 - RNA, 25 nt, synthetic construct
  misc_feature 1..25: note = oligonucleotide
  source 1..25: mol_type = other RNA
tccttagcttccagccattgtgttg

SEQ ID NO: 344 - RNA, 25 nt, synthetic construct
  misc_feature 1..25: note = oligonucleotide
  source 1..25: mol_type = other RNA
ccttagcttccagccattgtgttga

SEQ ID NO: 345 - RNA, 25 nt, synthetic construct
  misc_feature 1..25: note = oligonucleotide
  source 1..25: mol_type = other RNA
cttagcttccagccattgtgttgaa

SEQ ID NO: 346 - RNA, 25 nt, synthetic construct
  misc_feature 1..25: note = oligonucleotide
  source 1..25: mol_type = other RNA
ttagcttccagccattgtgttgaat

SEQ ID NO: 347 - RNA, 25 nt, synthetic construct
  misc_feature 1..25: note = oligonucleotide
  source 1..25: mol_type = other RNA
tagcttccagccattgtgttgaatc

SEQ ID NO: 348 - RNA, 25 nt, synthetic construct
  misc_feature 1..25: note = oligonucleotide
  source 1..25: mol_type = other RNA
agcttccagccattgtgttgaatcc

SEQ ID NO: 349 - RNA, 25 nt, synthetic construct
  misc_feature 1..25: note = oligonucleotide
  source 1..25: mol_type = other RNA
gcttccagccattgtgttgaatcct

SEQ ID NO: 350 - RNA, 25 nt, synthetic construct
  misc_feature 1..25: note = oligonucleotide
  source 1..25: mol_type = other RNA
cttccagccattgtgttgaatcctt

SEQ ID NO: 351 - RNA, 25 nt, synthetic construct
  misc_feature 1..25: note = oligonucleotide
  source 1..25: mol_type = other RNA
ttccagccattgtgttgaatccttt

SEQ ID NO: 352 - RNA, 25 nt, synthetic construct
  misc_feature 1..25: note = oligonucleotide
  source 1..25: mol_type = other RNA
tccagccattgtgttgaatccttta

SEQ ID NO: 353 - RNA, 25 nt, synthetic construct
  misc_feature 1..25: note = oligonucleotide
  source 1..25: mol_type = other RNA
ccagccattgtgttgaatcctttaa

SEQ ID NO: 354 - RNA, 25 nt, synthetic construct
  misc_feature 1..25: note = oligonucleotide
  source 1..25: mol_type = other RNA
cagccattgtgttgaatcctttaac

SEQ ID NO: 355 - RNA, 25 nt, synthetic construct
  misc_feature 1..25: note = oligonucleotide
  source 1..25: mol_type = other RNA
agccattgtgttgaatcctttaaca

SEQ ID NO: 356 - RNA, 25 nt, synthetic construct
  misc_feature 1..25: note = oligonucleotide
  source 1..25: mol_type = other RNA
gccattgtgttgaatcctttaacat

SEQ ID NO: 357 - RNA, 25 nt, synthetic construct
  misc_feature 1..25: note = oligonucleotide
  source 1..25: mol_type = other RNA
ccattgtgttgaatcctttaacatt

SEQ ID NO: 358 - RNA, 25 nt, synthetic construct
  misc_feature 1..25: note = oligonucleotide
  source 1..25: mol_type = other RNA
cattgtgttgaatcctttaacattt

SEQ ID NO: 359 - RNA, 20 nt, synthetic construct
  misc_feature 1..20: note = oligonucleotide
  source 1..20: mol_type = other RNA
catttttgacctacatgtgg

SEQ ID NO: 360 - RNA, 20 nt, synthetic construct
  misc_feature 1..20: note = oligonucleotide
  source 1..20: mol_type = other RNA
tttgacctacatgtggaaag

SEQ ID NO: 361 - RNA, 26 nt, synthetic construct
  misc_feature 1..26: note = oligonucleotide
  source 1..26: mol_type = other RNA
tacatttttgacctacatgtggaaag

SEQ ID NO: 362 - RNA, 17 nt, synthetic construct
  misc_feature 1..17: note = oligonucleotide
  source 1..17: mol_type = other RNA
ggtctccttacctatga

SEQ ID NO: 363 - RNA, 24 nt, synthetic construct
  misc_feature 1..24: note = oligonucleotide
  source 1..24: mol_type = other RNA
tcttacctatgactatggatgaga

SEQ ID NO: 364 - RNA, 22 nt, synthetic construct
  misc_feature 1..22: note = oligonucleotide
  source 1..22: mol_type = other RNA
atttttgacctacatgggaaag

SEQ ID NO: 365 - RNA, 23 nt, synthetic construct
  misc_feature 1..23: note = oligonucleotide
  source 1..23: mol_type = other RNA
tacgagttgattgtcggacccag

SEQ ID NO: 366 - RNA, 25 nt, synthetic construct
  misc_feature 1..25: note = oligonucleotide
  source 1..25: mol_type = other RNA
gtggtctccttacctatgactgtgg

SEQ ID NO: 367 - RNA, 24 nt, synthetic construct
  misc_feature 1..24: note = oligonucleotide
  source 1..24: mol_type = other RNA
tgtctcagtaatcttcttacctat

SEQ ID NO: 368 - RNA, 23 nt, synthetic construct
  misc_feature 1..23: note = oligonucleotide
  source 1..23: mol_type = other RNA
tgcatgttccagtcgttgtgtgg

SEQ ID NO: 369 - RNA, 25 nt, synthetic construct
  misc_feature 1..25: note = oligonucleotide
  source 1..25: mol_type = other RNA
cactattccagtcaaataggtctgg

SEQ ID NO: 370 - RNA, 25 nt, synthetic construct
  misc_feature 1..25: note = oligonucleotide
  source 1..25: mol_type = other RNA
atttaccaaccttcaggatcgagta

SEQ ID NO: 371 - RNA, 21 nt, synthetic construct
  misc_feature 1..21: note = oligonucleotide
  source 1..21: mol_type = other RNA
ggcctaaaacacatacacata

SEQ ID NO: 372 - RNA, 24 nt, synthetic construct
  misc_feature 1..24: note = oligonucleotide
  source 1..24: mol_type = other RNA
ccctgaggcattcccatcttgaat

SEQ ID NO: 373 - RNA, 20 nt, synthetic construct
  misc_feature 1..20: note = oligonucleotide
  source 1..20: mol_type = other RNA
aggacttacttgctttgttt

SEQ ID NO: 374 - RNA, 23 nt, synthetic construct
  misc_feature 1..23: note = oligonucleotide
  source 1..23: mol_type = other RNA
cttgaatttaggagattcatctg

SEQ ID NO: 375 - RNA, 23 nt, synthetic construct
  misc_feature 1..23: note = oligonucleotide
  source 1..23: mol_type = other RNA
catcttctgataattttcctgtt

SEQ ID NO: 376 - RNA, 20 nt, synthetic construct
  misc_feature 1..20: note = oligonucleotide
  source 1..20: mol_type = other RNA
ccattacagttgtctgtgtt

SEQ ID NO: 377 - RNA, 23 nt, synthetic construct
  misc_feature 1..23: note = oligonucleotide
  source 1..23: mol_type = other RNA
tgacagcctgtgaaatctgtgag

SEQ ID NO: 378 - RNA, 20 nt, synthetic construct
  misc_feature 1..20: note = oligonucleotide
  source 1..20: mol_type = other RNA
taatctgcctcttcttttgg

SEQ ID NO: 379 - RNA, 20 nt, synthetic construct
  misc_feature 1..20: note = oligonucleotide
  source 1..20: mol_type = other RNA
cagcagtagttgtcatctgc

SEQ ID NO: 380 - RNA, 27 nt, synthetic construct
  misc_feature 1..27: note = oligonucleotide
  source 1..27: mol_type = other RNA
gcctgagctgatctgctggcatcttgc

SEQ ID NO: 381 - RNA, 31 nt, synthetic construct
  misc_feature 1..31: note = oligonucleotide
  source 1..31: mol_type = other RNA
gcctgagctgatctgctggcatcttgcagtt

SEQ ID NO: 382 - RNA, 16 nt, synthetic construct
  misc_feature 1..16: note = oligonucleotide
  source 1..16: mol_type = other RNA
tctgctggcatcttgc

SEQ ID NO: 383 - RNA, 22 nt, synthetic construct
  misc_feature 1..22: note = oligonucleotide
  source 1..22: mol_type = other RNA
gccggttgacttcatcctgtgc

SEQ ID NO: 384 - RNA, 23 nt, synthetic construct
  misc_feature 1..23: note = oligonucleotide
  source 1..23: mol_type = other RNA
gtctgcatccaggaacatgggtc

SEQ ID NO: 385 - RNA, 24 nt, synthetic construct
  misc_feature 1..24: note = oligonucleotide
  source 1..24: mol_type = other RNA
tacttactgtctgtagctctttct

SEQ ID NO: 386 - RNA, 22 nt, synthetic construct
  misc_feature 1..22: note = oligonucleotide
  source 1..22: mol_type = other RNA
ctgcatccaggaacatgggtcc

SEQ ID NO: 387 - RNA, 24 nt, synthetic construct
  misc_feature 1..24: note = oligonucleotide
  source 1..24: mol_type = other RNA
gttgaagatctgatagccggttga

SEQ ID NO: 388 - RNA, 20 nt, synthetic construct
  misc_feature 1..20: note = oligonucleotide
  source 1..20: mol_type = other RNA
tcagcttctgttagccactg

SEQ ID NO: 389 - RNA, 20 nt, synthetic construct
  misc_feature 1..20: note = oligonucleotide
  source 1..20: mol_type = other RNA
ttcagcttctgttagccact

SEQ ID NO: 390 - RNA, 21 nt, synthetic construct
  misc_feature 1..21: note = oligonucleotide
  source 1..21: mol_type = other RNA
ttcagcttctgttagccactg

SEQ ID NO: 391 - RNA, 21 nt, synthetic construct
  misc_feature 1..21: note = oligonucleotide
  source 1..21: mol_type = other RNA
tcagcttctgttagccactga

SEQ ID NO: 392 - RNA, 22 nt, synthetic construct
  misc_feature 1..22: note = oligonucleotide
  source 1..22: mol_type = other RNA
ttcagcttctgttagccactga

SEQ ID NO: 393 - RNA, 21 nt, synthetic construct
  misc_feature 1..21: note = oligonucleotide
  source 1..21: mol_type = other RNA
tcagcttctgttagccactga

SEQ ID NO: 394 - RNA, 22 nt, synthetic construct
  misc_feature 1..22: note = oligonucleotide
  source 1..22: mol_type = other RNA
ttcagcttctgttagccactga

SEQ ID NO: 395 - RNA, 22 nt, synthetic construct
  misc_feature 1..22: note = oligonucleotide
  source 1..22: mol_type = other RNA
tcagcttctgttagccactgat

SEQ ID NO: 396 - RNA, 23 nt, synthetic construct
  misc_feature 1..23: note = oligonucleotide
  source 1..23: mol_type = other RNA
ttcagcttctgttagccactgat

SEQ ID NO: 397 - RNA, 23 nt, synthetic construct
  misc_feature 1..23: note = oligonucleotide
  source 1..23: mol_type = other RNA
tcagcttctgttagccactgatt

SEQ ID NO: 398 - RNA, 24 nt, synthetic construct
  misc_feature 1..24: note = oligonucleotide
  source 1..24: mol_type = other RNA
ttcagcttctgttagccactgatt

SEQ ID NO: 399 - RNA, 24 nt, synthetic construct
  misc_feature 1..24: note = oligonucleotide
  source 1..24: mol_type = other RNA
tcagcttctgttagccactgatta

SEQ ID NO: 400 - RNA, 24 nt, synthetic construct
  misc_feature 1..24: note = oligonucleotide
  source 1..24: mol_type = other RNA
ttcagcttctgttagccactgata

SEQ ID NO: 401 - RNA, 25 nt, synthetic construct
  misc_feature 1..25: note = oligonucleotide
  source 1..25: mol_type = other RNA
tcagcttctgttagccactgattaa

SEQ ID NO: 402 - RNA, 26 nt, synthetic construct
  misc_feature 1..26: note = oligonucleotide
  source 1..26: mol_type = other RNA
ttcagcttctgttagccactgattaa

SEQ ID NO: 403 - RNA, 26 nt, synthetic construct
  misc_feature 1..26: note = oligonucleotide
  source 1..26: mol_type = other RNA
tcagcttctgttagccactgattaaa

SEQ ID NO: 404 - RNA, 27 nt, synthetic construct
  misc_feature 1..27: note = oligonucleotide
  source 1..27: mol_type = other RNA
ttcagcttctgttagccactgattaaa

SEQ ID NO: 405 - RNA, 19 nt, synthetic construct
  misc_feature 1..19: note = oligonucleotide
  source 1..19: mol_type = other RNA
cagcttctgttagccactg

SEQ ID NO: 406 - RNA, 21 nt, synthetic construct
  misc_feature 1..21: note = oligonucleotide
  source 1..21: mol_type = other RNA
cagcttctgttagccactgat

SEQ ID NO: 407 - RNA, 21 nt, synthetic construct
  misc_feature 1..21: note = oligonucleotide
  source 1..21: mol_type = other RNA
agcttctgttagccactgatt

SEQ ID NO: 408 - RNA, 22 nt, synthetic construct
  misc_feature 1..22: note = oligonucleotide
  source 1..22: mol_type = other RNA
cagcttctgttagccactgatt

SEQ ID NO: 409 - RNA, 22 nt, synthetic construct
  misc_feature 1..22: note = oligonucleotide
  source 1..22: mol_type = other RNA
agcttctgttagccactgatta

SEQ ID NO: 410 - RNA, 23 nt, synthetic construct
  misc_feature 1..23: note = oligonucleotide
  source 1..23: mol_type = other RNA
cagcttctgttagccactgatta

SEQ ID NO: 411 - RNA, 23 nt, synthetic construct
  misc_feature 1..23: note = oligonucleotide
  source 1..23: mol_type = other RNA
agcttctgttagccactgattaa

SEQ ID NO: 412 - RNA, 24 nt, synthetic construct
  misc_feature 1..24: note = oligonucleotide
  source 1..24: mol_type = other RNA
cagcttctgttagccactgattaa

SEQ ID NO: 413 - RNA, 24 nt, synthetic construct
  misc_feature 1..24: note = oligonucleotide
  source 1..24: mol_type = other RNA
agcttctgttagccactgattaaa

SEQ ID NO: 414 - RNA, 25 nt, synthetic construct
  misc_feature 1..25: note = oligonucleotide
  source 1..25: mol_type = other RNA
cagcttctgttagccactgattaaa

SEQ ID NO: 415 - RNA, 24 nt, synthetic construct
  misc_feature 1..24: note = oligonucleotide
  source 1..24: mol_type = other RNA
agcttctgttagccactgattaaa

SEQ ID NO: 416 - RNA, 20 nt, synthetic construct
  misc_feature 1..20: note = oligonucleotide
  source 1..20: mol_type = other RNA
agcttctgttagccactgat

SEQ ID NO: 417 - RNA, 20 nt, synthetic construct
  misc_feature 1..20: note = oligonucleotide
  source 1..20: mol_type = other RNA
gcttctgttagccactgatt

SEQ ID NO: 418 - RNA, 21 nt, synthetic construct
  misc_feature 1..21: note = oligonucleotide
  source 1..21: mol_type = other RNA
agcttctgttagccactgatt

SEQ ID NO: 419 - RNA, 21 nt, synthetic construct
  misc_feature 1..21: note = oligonucleotide
  source 1..21: mol_type = other RNA
gcttctgttagccactgatta

SEQ ID NO: 420 - RNA, 22 nt, synthetic construct
  misc_feature 1..22: note = oligonucleotide
  source 1..22: mol_type = other RNA
agcttctgttagccactgatta

SEQ ID NO: 421 - RNA, 22 nt, synthetic construct
  misc_feature 1..22: note = oligonucleotide
  source 1..22: mol_type = other RNA
gcttctgttagccactgattaa

SEQ ID NO: 422 - RNA, 23 nt, synthetic construct
  misc_feature 1..23: note = oligonucleotide
  source 1..23: mol_type = other RNA
agcttctgttagccactgattaa

SEQ ID NO: 423 - RNA, 23 nt, synthetic construct
  misc_feature 1..23: note = oligonucleotide
  source 1..23: mol_type = other RNA
gcttctgttagccactgattaaa

SEQ ID NO: 424 - RNA, 24 nt, synthetic construct
  misc_feature 1..24: note = oligonucleotide
  source 1..24: mol_type = other RNA
agcttctgttagccactgattaaa

SEQ ID NO: 425 - RNA, 23 nt, synthetic construct
  misc_feature 1..23: note = oligonucleotide
  source 1..23: mol_type = other RNA
gcttctgttagccactgattaaa

SEQ ID NO: 426 - RNA, 23 nt, synthetic construct
  misc_feature 1..23: note = oligonucleotide
  source 1..23: mol_type = other RNA
ccatttgtatttagcatgttccc

SEQ ID NO: 427 - RNA, 20 nt, synthetic construct
  misc_feature 1..20: note = oligoucleotide
  source 1..20: mol_type = other RNA
agataccatttgtatttagc

SEQ ID NO: 428 - RNA, 19 nt, synthetic construct
  misc_feature 1..19: note = oligonucleotide
  source 1..19: mol_type = other RNA
gccatttctcaacagatct

SEQ ID NO: 429 - RNA, 23 nt, synthetic construct
  misc_feature 1..23: note = oligonucleotide
  source 1..23: mol_type = other RNA
gccatttctcaacagatctgtca

SEQ ID NO: 430 - RNA, 23 nt, synthetic construct
  misc_feature 1..23: note = oligonucleotide
  source 1..23: mol_type = other RNA
attctcaggaatttgtgtctttc

SEQ ID NO: 431 - RNA, 21 nt, synthetic construct
  misc_feature 1..21: note = oligonucleotide
  source 1..21: mol_type = other RNA
tctcaggaatttgtgtctttc

SEQ ID NO: 432 - RNA, 18 nt, synthetic construct
  misc_feature 1..18: note = oligonucleotide
  source 1..18: mol_type = other RNA
gttcagcttctgttagcc

SEQ ID NO: 433 - RNA, 21 nt, synthetic construct
  misc_feature 1..21: note = oligonucleotide
  source 1..21: mol_type = other RNA
ctgattaaatatctttatatc

SEQ ID NO: 434 - RNA, 18 nt, synthetic construct
  misc_feature 1..18: note = oligonucleotide
  source 1..18: mol_type = other RNA
gccgccatttctcaacag

SEQ ID NO: 435 - RNA, 18 nt, synthetic construct
  misc_feature 1..18: note = oligonucleotide
  source 1..18: mol_type = other RNA
gccgccatttctcaacag

SEQ ID NO: 436 - RNA, 18 nt, synthetic construct
  misc_feature 1..18: note = oligonucleotide
  source 1..18: mol_type = other RNA
caggaatttgtgtctttc

SEQ ID NO: 437 - RNA, 25 nt, synthetic construct
  misc_feature 1..25: note = oligonucleotide
  source 1..25: mol_type = other RNA
tttgccgctgcccaatgccatcctg

SEQ ID NO: 438 - RNA, 25 nt, synthetic construct
  misc_feature 1..25: note = oligonucleotide
  source 1..25: mol_type = other RNA
attcaatgttctgacaacagtttgc

SEQ ID NO: 439 - RNA, 25 nt, synthetic construct
  misc_feature 1..25: note = oligonucleotide
  source 1..25: mol_type = other RNA
ccagttgcattcaatgttctgacaa

SEQ ID NO: 440 - RNA, 22 nt, synthetic construct
  misc_feature 1..22: note = oligonucleotide
  source 1..22: mol_type = other RNA
cagttgcattcaatgttctgac

SEQ ID NO: 441 - RNA, 20 nt, synthetic construct
  misc_feature 1..20: note = oligonucleotide
  source 1..20: mol_type = other RNA
agttgcattcaatgttctga

SEQ ID NO: 442 - RNA, 21 nt, synthetic construct
  misc_feature 1..21: note = oligonucleotide
  source 1..21: mol_type = other RNA
gattgctgaattatttcttcc

SEQ ID NO: 443 - RNA, 25 nt, synthetic construct
  misc_feature 1..25: note = oligonucleotide
  source 1..25: mol_type = other RNA
gattgctgaattatttcttccccag

SEQ ID NO: 444 - RNA, 25 nt, synthetic construct
  misc_feature 1..25: note = oligonucleotide
  source 1..25: mol_type = other RNA
attgctgaattatttcttccccagt

SEQ ID NO: 445 - RNA, 25 nt, synthetic construct
  misc_feature 1..25: note = oligonucleotide
  source 1..25: mol_type = other RNA
ttgctgaattatttcttccccagtt

SEQ ID NO: 446 - RNA, 25 nt, synthetic construct
  misc_feature 1..25: note = oligonucleotide
  source 1..25: mol_type = other RNA
tgctgaattatttcttccccagttg

SEQ ID NO: 447 - RNA, 25 nt, synthetic construct
  misc_feature 1..25: note = oligonucleotide
  source 1..25: mol_type = other RNA
gctgaattatttcttccccagttgc

SEQ ID NO: 448 - RNA, 25 nt, synthetic construct
  misc_feature 1..25: note = oligonucleotide
  source 1..25: mol_type = other RNA
ctgaattatttcttccccagttgca

SEQ ID NO: 449 - RNA, 25 nt, synthetic construct
  misc_feature 1..25: note = oligonucleotide
  source 1..25: mol_type = other RNA
tgaattatttcttccccagttgcat

SEQ ID NO: 450 - RNA, 25 nt, synthetic construct
  misc_feature 1..25: note = oligonucleotide
  source 1..25: mol_type = other RNA
gaattatttcttccccagttgcatt

SEQ ID NO: 451 - RNA, 25 nt, synthetic construct
  misc_feature 1..25: note = oligonucleotide
  source 1..25: mol_type = other RNA
aattatttcttccccagttgcattc

SEQ ID NO: 452 - RNA, 25 nt, synthetic construct
  misc_feature 1..25: note = oligonucleotide
  source 1..25: mol_type = other RNA
attatttcttccccagttgcattca

SEQ ID NO: 453 - RNA, 25 nt, synthetic construct
  misc_feature 1..25: note = oligonucleotide
  source 1..25: mol_type = other RNA
ttatttcttccccagttgcattcaa

SEQ ID NO: 454 - RNA, 25 nt, synthetic construct
  misc_feature 1..25: note = oligonucleotide
  source 1..25: mol_type = other RNA
tatttcttccccagttgcattcaat

SEQ ID NO: 455 - RNA, 25 nt, synthetic construct
  misc_feature 1..25: note = oligonucleotide
  source 1..25: mol_type = other RNA
atttcttccccagttgcattcaatg

SEQ ID NO: 456 - RNA, 25 nt, synthetic construct
  misc_feature 1..25: note = oligonucleotide
  source 1..25: mol_type = other RNA
tttcttccccagttgcattcaatgt

SEQ ID NO: 457 - RNA, 25 nt, synthetic construct
  misc_feature 1..25: note = oligonucleotide
  source 1..25: mol_type = other RNA
ttcttccccagttgcattcaatgtt

SEQ ID NO: 458 - RNA, 25 nt, synthetic construct
  misc_feature 1..25: note = oligonucleotide
  source 1..25: mol_type = other RNA
tcttccccagttgcattcaatgttc

SEQ ID NO: 459 - RNA, 25 nt, synthetic construct
  misc_feature 1..25: note = oligonucleotide
  source 1..25: mol_type = other RNA
cttccccagttgcattcaatgttct

SEQ ID NO: 460 - RNA, 25 nt, synthetic construct
  misc_feature 1..25: note = oligonucleotide
  source 1..25: mol_type = other RNA
ttccccagttgcattcaatgttctg

SEQ ID NO: 461 - RNA, 25 nt, synthetic construct
  misc_feature 1..25: note = oligonucleotide
  source 1..25: mol_type = other RNA
tccccagttgcattcaatgttctga

SEQ ID NO: 462 - RNA, 25 nt, synthetic construct
  misc_feature 1..25: note = oligonucleotide
  source 1..25: mol_type = other RNA
ccccagttgcattcaatgttctgac

SEQ ID NO: 463 - RNA, 25 nt, synthetic construct
  misc_feature 1..25: note = oligonucleotide
  source 1..25: mol_type = other RNA
cccagttgcattcaatgttctgaca

SEQ ID NO: 464 - RNA, 25 nt, synthetic construct
  misc_feature 1..25: note = oligonucleotide
  source 1..25: mol_type = other RNA
ccagttgcattcaatgttctgacaa

SEQ ID NO: 465 - RNA, 25 nt, synthetic construct
  misc_feature 1..25: note = oligonucleotide
  source 1..25: mol_type = other RNA
cagttgcattcaatgttctgacaac

SEQ ID NO: 466 - RNA, 25 nt, synthetic construct
  misc_feature 1..25: note = oligonucleotide
  source 1..25: mol_type = other RNA
agttgcattcaatgttctgacaaca

SEQ ID NO: 467 - RNA, 20 nt, synthetic construct
  misc_feature 1..20: note = oligonucleotide
  source 1..20: mol_type = other RNA
tcctgtagaatactggcatc

SEQ ID NO: 468 - RNA, 27 nt, synthetic construct
  misc_feature 1..27: note = oligonucleotide
  source 1..27: mol_type = other RNA
tgcagacctcctgccaccgcagattca

SEQ ID NO: 469 - RNA, 34 nt, synthetic construct
  misc_feature 1..34: note = oligonucleotide
  source 1..34: mol_type = other RNA
ttgcagacctcctgccaccgcagattcaggcttc

SEQ ID NO: 470 - RNA, 25 nt, synthetic construct
  misc_feature 1..25: note = oligonucleotide
  source 1..25: mol_type = other RNA
gttgcattcaatgttctgacaacag

SEQ ID NO: 471 - RNA, 25 nt, synthetic construct
  misc_feature 1..25: note = oligonucleotide
  source 1..25: mol_type = other RNA
ttgcattcaatgttctgacaacagt

SEQ ID NO: 472 - RNA, 25 nt, synthetic construct
  misc_feature 1..25: note = oligonucleotide
  source 1..25: mol_type = other RNA
tgcattcaatgttctgacaacagtt

SEQ ID NO: 473 - RNA, 25 nt, synthetic construct
  misc_feature 1..25: note = oligonucleotide
  source 1..25: mol_type = other RNA
gcattcaatgttctgacaacagttt

SEQ ID NO: 474 - RNA, 25 nt, synthetic construct
  misc_feature 1..25: note = oligonucleotide
  source 1..25: mol_type = other RNA
cattcaatgttctgacaacagtttg

SEQ ID NO: 475 - RNA, 25 nt, synthetic construct
  misc_feature 1..25: note = oligonucleotide
  source 1..25: mol_type = other RNA
attcaatgttctgacaacagtttgc

SEQ ID NO: 476 - RNA, 25 nt, synthetic construct
  misc_feature 1..25: note = oligonucleotide
  source 1..25: mol_type = other RNA
tcaatgttctgacaacagtttgccg

SEQ ID NO: 477 - RNA, 25 nt, synthetic construct
  misc_feature 1..25: note = oligonucleotide
  source 1..25: mol_type = other RNA
caatgttctgacaacagtttgccgc

SEQ ID NO: 478 - RNA, 25 nt, synthetic construct
  misc_feature 1..25: note = oligonucleotide
  source 1..25: mol_type = other RNA
aatgttctgacaacagtttgccgct

SEQ ID NO: 479 - RNA, 25 nt, synthetic construct
  misc_feature 1..25: note = oligonucleotide
  source 1..25: mol_type = other RNA
atgttctgacaacagtttgccgctg

SEQ ID NO: 480 - RNA, 25 nt, synthetic construct
  misc_feature 1..25: note = oligonucleotide
  source 1..25: mol_type = other RNA
tgttctgacaacagtttgccgctgc

SEQ ID NO: 481 - RNA, 25 nt, synthetic construct
  misc_feature 1..25: note = oligonucleotide
  source 1..25: mol_type = other RNA
gttctgacaacagtttgccgctgcc

SEQ ID NO: 482 - RNA, 25 nt, synthetic construct
  misc_feature 1..25: note = oligonucleotide
  source 1..25: mol_type = other RNA
ttctgacaacagtttgccgctgccc

SEQ ID NO: 483 - RNA, 25 nt, synthetic construct
  misc_feature 1..25: note = oligonucleotide
  source 1..25: mol_type = other RNA
tctgacaacagtttgccgctgccca

SEQ ID NO: 484 - RNA, 25 nt, synthetic construct
  misc_feature 1..25: note = oligonucleotide
  source 1..25: mol_type = other RNA
ctgacaacagtttgccgctgcccaa

SEQ ID NO: 485 - RNA, 25 nt, synthetic construct
  misc_feature 1..25: note = oligonucleotide
  source 1..25: mol_type = other RNA
tgacaacagtttgccgctgcccaat

SEQ ID NO: 486 - RNA, 25 nt, synthetic construct
  misc_feature 1..25: note = oligonucleotide
  source 1..25: mol_type = other RNA
gacaacagtttgccgctgcccaatg

SEQ ID NO: 487 - RNA, 25 nt, synthetic construct
  misc_feature 1..25: note = oligonucleotide
  source 1..25: mol_type = other RNA
acaacagtttgccgctgcccaatgc

SEQ ID NO: 488 - RNA, 25 nt, synthetic construct
  misc_feature 1..25: note = oligonculeotide
  source 1..25: mol_type = other RNA
caacagtttgccgctgcccaatgcc

SEQ ID NO: 489 - RNA, 25 nt, synthetic construct
  misc_feature 1..25: note = oligonucleotide
  source 1..25: mol_type = other RNA
aacagtttgccgctgcccaatgcca

SEQ ID NO: 490 - RNA, 25 nt, synthetic construct
  misc_feature 1..25: note = oligonucleotide
  source 1..25: mol_type = other RNA
acagtttgccgctgcccaatgccat

SEQ ID NO: 491 - RNA, 25 nt, synthetic construct
  misc_feature 1..25: note = oligonucleotide
  source 1..25: mol_type = other RNA
cagtttgccgctgcccaatgccatc

SEQ ID NO: 492 - RNA, 25 nt, synthetic construct
  misc_feature 1..25: note = oligonucleotide
  source 1..25: mol_type = other RNA
agtttgccgctgcccaatgccatcc

SEQ ID NO: 493 - RNA, 25 nt, synthetic construct
  misc_feature 1..25: note = oligonucleotide
  source 1..25: mol_type = other RNA
gtttgccgctgcccaatgccatcct

SEQ ID NO: 494 - RNA, 25 nt, synthetic construct
  misc_feature 1..25: note = oligonucleotide
  source 1..25: mol_type = other RNA
tttgccgctgcccaatgccatcctg

SEQ ID NO: 495 - RNA, 25 nt, synthetic construct
  misc_feature 1..25: note = oligonucleotide
  source 1..25: mol_type = other RNA
ttgccgctgcccaatgccatcctgg

SEQ ID NO: 496 - RNA, 25 nt, synthetic construct
  misc_feature 1..25: note = oligonucleotide
  source 1..25: mol_type = other RNA
tgccgctgcccaatgccatcctgga

SEQ ID NO: 497 - RNA, 25 nt, synthetic construct
  misc_feature 1..25: note = oligonucleotide
  source 1..25: mol_type = other RNA
gccgctgcccaatgccatcctggag

SEQ ID NO: 498 - RNA, 25 nt, synthetic construct
  misc_feature 1..25: note = oligonucleotide
  source 1..25: mol_type = other RNA
ccgctgcccaatgccatcctggagt

SEQ ID NO: 499 - RNA, 25 nt, synthetic construct
  misc_feature 1..25: note = oligonucleotide
  source 1..25: mol_type = other RNA
cgctgcccaatgccatcctggagtt

SEQ ID NO: 500 - RNA, 20 nt, synthetic construct
  misc_feature 1..20: note = oligonucleotide
  source 1..20: mol_type = other RNA
tgtttttgaggattgctgaa

SEQ ID NO: 501 - RNA, 40 nt, synthetic construct
  misc_feature 1..40: note = oligonucleotide
  source 1..40: mol_type = other RNA
tgttctgacaacagtttgccgctgcccaatgccatcctgg

SEQ ID NO: 502 - RNA, 20 nt, synthetic construct
  misc_feature 1..20: note = oligonucleotide
  source 1..20: mol_type = other RNA
ctgttgcagtaatctatgag

SEQ ID NO: 503 - RNA, 20 nt, synthetic construct
  misc_feature 1..20: note = oligonucleotide
  source 1..20: mol_type = other RNA
tgcagtaatctatgagtttc

SEQ ID NO: 504 - RNA, 18 nt, synthetic construct
  misc_feature 1..18: note = oligonucleotide
  source 1..18: mol_type = other RNA
gagtcttctaggagcctt

SEQ ID NO: 505 - RNA, 23 nt, synthetic construct
  misc_feature 1..23: note = oligonucleotide
  source 1..23: mol_type = other RNA
tgccattgtttcatcagctcttt

SEQ ID NO: 506 - RNA, 20 nt, synthetic construct
  misc_feature 1..20: note = oligonucleotide
  source 1..20: mol_type = other RNA
tcctgtaggacattggcagt

SEQ ID NO: 507 - RNA, 20 nt, synthetic construct
  misc_feature 1..20: note = oligonucleotide
  source 1..20: mol_type = other RNA
cttggagtcttctaggagcc

SEQ ID NO: 508 - RNA, 17 nt, synthetic construct
  misc_feature 1..17: note = oligonucleotide
  source 1..17: mol_type = other RNA
taggtgcctgccggctt

SEQ ID NO: 509 - RNA, 18 nt, synthetic construct
  misc_feature 1..18: note = oligonucleotide
  source 1..18: mol_type = other RNA
ttcagctgtagccacacc

SEQ ID NO: 510 - RNA, 21 nt, synthetic construct
  misc_feature 1..21: note = oligonucleotide
  source 1..21: mol_type = other RNA
ctgaactgctggaaagtcgcc

SEQ ID NO: 511 - RNA, 30 nt, synthetic construct
  misc_feature 1..30: note = oligonucleotide
  source 1..30: mol_type = other RNA
ctggcttccaaatgggacctgaaaaagaac

SEQ ID NO: 512 - RNA, 21 nt, synthetic construct
  misc_feature 1..21: note = oligonucleotide
  source 1..21: mol_type = other RNA
caatttttcccactcagtatt

SEQ ID NO: 513 - RNA, 19 nt, synthetic construct
  misc_feature 1..19: note = oligonucleotide
  source 1..19: mol_type = other RNA
ttgaagttcctggagtctt

SEQ ID NO: 514 - RNA, 22 nt, synthetic construct
  misc_feature 1..22: note = oligonucleotide
  source 1..22: mol_type = other RNA
tcctcaggaggcagctctaaat

SEQ ID NO: 515 - RNA, 16 nt, synthetic construct
  misc_feature 1..16: note = oligonucleotide
  source 1..16: mol_type = other RNA
tggctctctcccaggg

SEQ ID NO: 516 - RNA, 27 nt, synthetic construct
  misc_feature 1..27: note = oligonucleotide
  source 1..27: mol_type = other RNA
gagatggctctctcccagggaccctgg

SEQ ID NO: 517 - RNA, 17 nt, synthetic construct
  misc_feature 1..17: note = oligonucleotide
  source 1..17: mol_type = other RNA
gggcactttgtttggcg

SEQ ID NO: 518 - RNA, 19 nt, synthetic construct
  misc_feature 1..19: note = oligonucleotide
  source 1..19: mol_type = other RNA
ggtcccagcaagttgtttg

SEQ ID NO: 519 - RNA, 25 nt, synthetic construct
  misc_feature 1..25: note = oligonucleotide
  source 1..25: mol_type = other RNA
tgggatggtcccagcaagttgtttg

SEQ ID NO: 520 - RNA, 21 nt, synthetic construct
  misc_feature 1..21: note = oligonucleotide
  source 1..21: mol_type = other RNA
gtagagctctgtcattttggg

SEQ ID NO: 521 - RNA, 25 nt, synthetic construct
  misc_feature 1..25: note = oligonucleotide
  source 1..25: mol_type = other RNA
gctcaagagatccactgcaaaaaac

SEQ ID NO: 522 - RNA, 26 nt, synthetic construct
  misc_feature 1..26: note = oligonucleotide
  source 1..26: mol_type = other RNA
gccatacgtacgtatcataaacattc

SEQ ID NO: 523 - RNA, 25 nt, synthetic construct
  misc_feature 1..25: note = oligonucleotide
  source 1..25: mol_type = other RNA
tctgcaggatatccatgggctggtc

SEQ ID NO: 524 - RNA, 27 nt, synthetic construct
  misc_feature 1..27: note = oligonucleotide
  source 1..27: mol_type = other RNA
gatcctccctgttcgtcccctattatg

SEQ ID NO: 525 - RNA, 24 nt, synthetic construct
  misc_feature 1..24: note = oligonucleotide
  source 1..24: mol_type = other RNA
tgctttagactcctgtacctgata

SEQ ID NO: 526 - RNA, 18 nt, synthetic construct
  misc_feature 1..18: note = oligonucleotide
  source 1..18: mol_type = other RNA
ggcggcctttgtgttgac

SEQ ID NO: 527 - RNA, 25 nt, synthetic construct
  misc_feature 1..25: note = oligonucleotide
  source 1..25: mol_type = other RNA
ggacaggcctttatgttcgtgctgc

SEQ ID NO: 528 - RNA, 19 nt, synthetic construct
  misc_feature 1..19: note = oligonucleotide
  source 1..19: mol_type = other RNA
cctttatgttcgtgctgct

SEQ ID NO: 529 - RNA, 25 nt, synthetic construct
  misc_feature 1..25: note = oligonucleotide
  source 1..25: mol_type = other RNA
cctcagctcttgaagtaaacggttt

SEQ ID NO: 530 - RNA, 25 nt, synthetic construct
  misc_feature 1..25: note = oligonucleotide
  source 1..25: mol_type = other RNA
ctcagctcttgaagtaaacggttta

SEQ ID NO: 531 - RNA, 25 nt, synthetic construct
  misc_feature 1..25: note = oligonucleotide
  source 1..25: mol_type = other RNA
tcagctcttgaagtaaacggtttac

SEQ ID NO: 532 - RNA, 25 nt, synthetic construct
  misc_feature 1..25: note = oligonucleotide
  source 1..25: mol_type = other RNA
cagctcttgaagtaaacggtttacc

SEQ ID NO: 533 - RNA, 25 nt, synthetic construct
  misc_feature 1..25: note = oligonucleotide
  source 1..25: mol_type = other RNA
agctcttgaagtaaacggtttaccg

SEQ ID NO: 534 - RNA, 25 nt, synthetic construct
  misc_feature 1..25: note = oligonucleotide
  source 1..25: mol_type = other RNA
gctcttgaagtaaacggtttaccgc

SEQ ID NO: 535 - RNA, 25 nt, synthetic construct
  misc_feature 1..25: note = oligonucleotide
  source 1..25: mol_type = other RNA
ctcttgaagtaaacggtttaccgcc